(19) United States
(12) Patent Application Publication
(10) Pub. No.: US 2007/0072811 A1
(43) Pub. Date: Mar. 29, 2007

(54) Bifunctional heterocyclic compounds and methods of making and using the same

(76) Inventors: Jay J. Farmer, New Haven, CT (US); Joyce A. Sutchliffe, Branford, CT (US); Ashoke Bhattacharjee, West Haven, CT (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/547,889

(22) PCT Filed: Mar. 5, 2004
(86) PCT No.: PCT/US04/06892

§ 371 Date: Aug. 14, 2006

Related U.S. Application Data
(60) Provisional application No. 60/451,951, filed on Mar. 5, 2003.

(51) Int. Cl. A61K 31/7008; A61K 31/7056; C07H 17/02

(52) U.S. Cl. 514/23; 514/62; 536/17.4; 536/18.7

(57) ABSTRACT

The invention provides a family of bifunctional heterocyclic compounds useful as antiinfective, anti-proliferative, anti-inflammatory, and prokinetic agents. The invention also provides methods of making the bifunctional heterocyclic compounds, and methods of using such compounds as anti-infective, anti-proliferative agents, anti-inflammatory, and/or prokinetic agents.

### RELATED APPLICATIONS

This application claims the benefit of and priority to U.S. Patent Application No. 60/451,951, filed Mar. 5, 2003, the disclosure of which is incorporated by reference herein.

### FIELD OF THE INVENTION

The present invention relates generally to the field of anti-infective and anti-proliferative agents. More particularly, the invention relates to a family of bifunctional heterocyclic compounds useful as such agents.

### BACKGROUND

Since the discovery of penicillin in the 1920s and streptomycin in the 1940s, many new compounds have been discovered or specifically designed for use as antibiotic agents. It was once believed that infectious diseases could be completely controlled or eradicated with the use of such therapeutic agents. However, such beliefs have been challenged by the fact that strains of microorganisms resistant to currently effective therapeutic agents continue to evolve. Almost every antibiotic agent developed for clinical use has encountered problems with the emergence of resistant bacteria. For example, resistant strains of Gram-positive bacteria such as methicillin-resistant staphylocci, penicillin-resistant streptococci, and vancomycin-resistant enterococci have developed, and can cause serious and often time fatal results for patients infected with such resistant bacteria Bacteria that are resistant to the macrolide antibiotics have developed. Also, Gram-negative strains of bacteria such as H. influenzae and M. catarrhalis have been identified. See, e.g., F. D. Lowry, Antimicrobial resistance: the example of Staphylococcus aureus, J. Clin. Invest., Vol. 111, No. 9, pp. 1265-1273 (2003); and Gold, H. S. and Moellering, R. C., Jr., Antimicrobial-drug resistance. N. Engl. J. Med., vol. 335, 1445-53 (1996).

This problem of resistance is not limited to the area of anti-infective agents, because resistance has also been encountered with anti-proliferative agents used in cancer chemotherapy. Therefore, the need exists to develop new anti-infective and anti-proliferative agents that are both effective against resistant bacteria and strains of cells and against which bacteria and strains of cells are less likely to develop resistance.

Despite this problem of increasing antibiotic resistance, no new major classes of antibiotics have been developed for clinical use since the approval in the United States in 2000 of the oxazolidinone ring-containing antibiotic, N-[[(5S)-3-[3-fluoro-4-(4-morpholinyl)phenyl]-2-oxo-5-oxazolidinyl]methyl acetamide (see structure 1), which is known as linezolid and which is sold under the tradename Zyvox® (see compound A). See, R. C. Moellering, Jr., Linezolid: The First Oxazolidinone Antimicrobial, Annals of Internal Medicine, Vol. 138, No. 2, pp. 135-142 (2003).

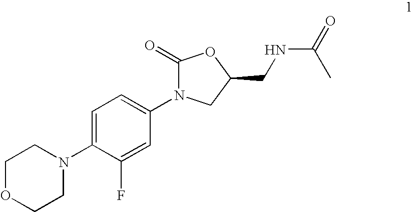

Linezolid was approved for use as an anti-bacterial agent active against Gram-positive organisms. However, linezolid-resistant strains of organisms are already being reported. See Tsiodras et al., Lancet, 2001, 358, 207; Gonzales et al., Lancet, 2001, 357, 1179; Zurenko et al., Proceedings Of The 39^{th} Annual Interscience Conference On Antibacterial Agents And Chemotherapy (ICAAC); San Francisco, Calif., USA, Sep. 26-29, 1999). However, investigators have been working to develop other effective linezolid derivatives. Research has indicated that the oxazolidinone ring could be important for linezolid's activity. The literature describes molecules having small groups substituted at the C-5 of the oxazolidinone ring, and early structure-activity relationships suggested that compounds with larger groups at the C-5 position were less active as anti-bacterial agents. As a consequence, investigators have been reluctant to place large substituents at the C-5 position of oxazolidinone rings in developing new anti-microbial agents.

Another class of antibiotics is the macrolides, which is so named for the 14- to 16-membered ring that is the major structural characteristic of this class of compounds. The first macrolide antibiotic to be developed was erythromycin, which was isolated from a soil sample from the Philippines in 1952. Even though erythromycin has been one of the most widely prescribed antibiotics, it has the disadvantages of relatively low bioavailability, gastrointestinal side effects, and a limited spectrum of activity. See Yong-Ji Wu, Highlights of Semi-synthetic Developments from Erythromycin A, Current Pharm. Design 6, pp. 181-223 (2000), and Yong-Ji Wu and Wei-uo Su, Recent Developments on Ketolides and Macrolides, Curr. Med. Chem., 8(14), pp. 1727-1758 (2001).

In the search for new therapeutic agents, pharmaceutical researchers have tried combining or linking various portions of antibiotic molecules. However, this approach has met with limited success.

U.S. Pat. No. 5,693,791, to Truett, issued Dec. 2, 1997 describes an antibiotic of the formula:
A-L-B
wherein A and B are antibiotics selected from the group consisting of sulfonamides, penicillins, cephalosporins, quinolones, chloramphenicol, erythromycin (i.e., a macrolide antibiotic), metronidzole, tetracyclines, and aminoglycosides. L is a linker formed from a difunctional linking agent.

PCT publication No. WO 99/63937, to Advanced Medicine, Inc., published Dec. 16, 1999, describes multi-binding compounds useful as antibiotics that are of the following formula:
(L)ₚ(X)_{q}
wherein L is selected from the group consisting of a macrolide antibiotic, an aminoglycoside, lincosamide, oxazolidinone, streptogramin, tetracycline, or another compound that binds to bacterial ribosomal RNA and/or to one or more proteins involved in ribosomal protein synthesis in the bacterium. P is an integer from 2-10. Q is an integer from 1-20. X is a linker.

U.S. Pat. No. 6,034,069, to Or et al., issued Mar. 7, 2000 depicts a series of 3′-N-modified 6-O-substituted erythromycin ketolide derivatives such structure 2 below. R, R¹, and R² are selected from the group consisting of a variety of groups, including aryl-alkoxy-heteroaryl-alkylene. R^{p} is H or a hydroxy protecting group. W is absent or is O, NH, or NCH₃. R^{w} is H or an optionally substituted alkyl group.

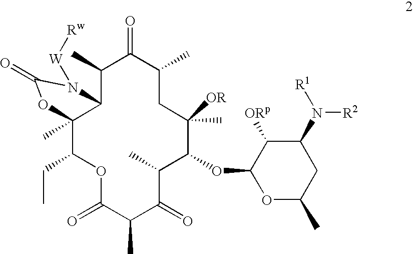

International patent publication No. WO 99/63937 proposes the synthesis of a large variety of multivalent macrolide antibiotics comprising a portion of a macrolide antibiotic linked via a linker to a portion of another known antibacterial agent. Compounds 3 and 4 below are two proposed compounds, although apparently neither was made or tested.

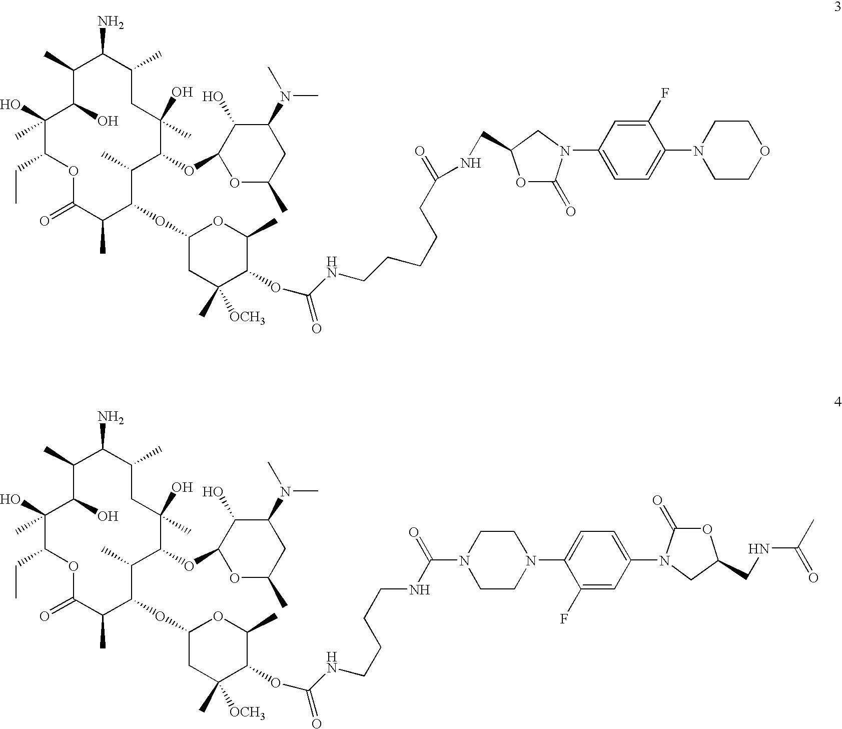

Notwithstanding the foregoing, there is an ongoing need for new anti-infective and anti-proliferative agents. Furthermore, because many anti-infective and anti-proliferative agents have utility as anti-inflammatory agents and also as prokinetic (gastrointestinal modulatory) agents, there is also an ongoing need for new compounds useful as anti-inflammatory and prokinetic agents.

### SUMMARY OF THE INVENTION

The invention provides a family of compounds useful as anti-infective agents and/or anti-proliferative agents, for example, chemotherapeutic agents, anti-fungal agents, anti-bacterial agents, anti-parasitic agents, anti-viral agents, having the formula: 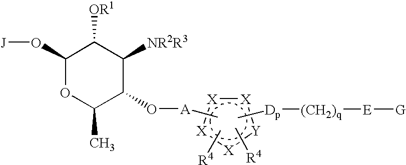
or pharmaceutically acceptable salts, esters, or prodrugs thereof. In the formula, p and q independently are 0 or 1. The variables A, D, E, G, J, R¹, R², R³, R⁴, X, and Y can be selected from the respective groups of chemical moieties later defined in the detailed description.

In addition, the invention provides methods of synthesizing the foregoing compounds. Following synthesis, the compounds may be formulated with a pharmaceutically acceptable carrier for administration to a mammal, fish, or fowl for use as an anti-cancer, anti-fungal, anti-bacterial, anti-parasitic, or anti-viral agent. In one embodiment, the compounds or the formulations may be used to treat microbial infections, for example, anti-bacterial or anti-fungal infections, in the mammal, fish, or fowl. Accordingly, the compounds or the formulations may be administered, for example, via oral, parenteral or topical routes, to provide an effective amount of the compound to the mammal, fish, or fowl.

The foregoing and other aspects and embodiments of the invention may be more fully understood by reference to the following detailed description and claims.

### DETAILED DESCRIPTION OF THE INVENTION

The present invention provides a family of compounds that can be used as anti-proliferative agents and/or anti-infective agents. The compounds may be used without limitation, for example, as anti-cancer agents, anti-bacterial agents, anti-fungal agents, anti-parasitic agents and/or anti-viral agents.

### 1. DEFINITIONS

For the purpose of the present invention, the following definitions have been used throughout.

The term “substituted,” as used herein, means that any one or more hydrogens on the designated atom is replaced with a selection from the indicated group, provided that the designated atom's normal valency is not exceeded, and that the substitution results in a stable compound. When a substituent is keto (i.e., ═O), then 2 hydrogens on the atom are replaced. Keto substituents are not present on aromatic moieties. Ring double bonds, as used herein, are double bonds that are formed between two adjacent ring atoms (e.g., C═C, C═N, or N═N).

The present invention is intended to include all isotopes of atoms occurring in the present compounds. Isotopes include those atoms having the same atomic number but different mass numbers. By way of general example and without limitation, isotopes of hydrogen include tritium and deuterium. Isotopes of carbon include C-13 and C-14.

When any variable (e.g., R³) occurs more than one time in any constituent or formula for a compound, its definition at each occurrence is independent of its definition at every other occurrence. Thus, for example, if a group is shown to be substituted with one or more R³ moieties, then the group may optionally be substituted with one, two, three, four, five, or more R³ moieties, and R³ at each occurrence is selected independently from the definition of R³. Also, combinations of substituents and/or variables are permissible, but only if such combinations result in stable compounds.

In the formulas herein, a broken or dashed circle within a ring indicates that the ring is either aromatic or non-aromatic. A bond extending from a chemical moiety that is depicted as crossing a bond in a ring, but is not attached directly to a ring atom, indicates that the chemical moiety may be bonded to any atom of the ring. When a substituent is listed without indicating the atom via which such substituent is bonded to the rest of the compound of a given formula, then such substituent may be bonded via any atom in such substituent. As to any of the above chemical moieties that contain one or more substituents, it is understood that such moieties do not contain any substitution or substitution patterns that are sterically impractical and/or synthetically unfeasible. In addition, the compounds of this invention include all stereochemical isomers arising from the substitution of these moieties.

As used herein, the terms used to describe various carbon-containing moieties, including, for example, “alkyl,” “alkenyl,” “allynyl,” “carbocycle,” and any variations thereof, are intended to include univalent, bivalent, or multivalent species. For example, “C₁₋₆ alkyl-R⁹” is intended to represent a univalent C₁₋₆ alkyl group substituted with a R³ group, and “O—C₁₋₆ alkyl-R³” is intended to represent a bivalent C₁₋₆ alkyl group, i.e., an “alkylene” group, substituted with an oxygen atom and a R³ group.

In cases wherein there are nitrogens in the compounds of the present invention, these can be converted to N-oxides by treatment with an oxidizing agent (e.g., MCPBA and/or hydrogen peroxides) to afford other compounds of the present invention. Thus, all shown and claimed nitrogens are considered to cover both the shown nitrogen and its N-oxide N→O) derivative.

As used herein, “alkyl” is intended to include both branched and straight-chain saturated aliphatic hydrocarbon groups having the specified number of carbon atoms. C₁₋₆ alkyl is intended to include C₁, C₂, C₃, C₄, C₅, and C₆ alkyl groups. C₁₋₈ alkyl is intended to include C₁, C₂, C₃, C₄, C₅, C₆, C₇, and C₈ alkyl groups. Examples of alkyl include, but are not limited to, methyl, ethyl, n-propyl, i-propyl, n-butyl, s-butyl, t-butyl, n-pentyl, s-pentyl, n-hexyl, n-heptyl, and n-octyl.

As used herein, “alkenyl” is intended to include hydrocarbon chains of either straight or branched configuration and one or more unsaturated carbon-carbon bonds that may occur in any stable point along the chain, such as ethenyl and propenyl. C₂₋₆ alkenyl is intended to include C₂, C₃, C₄, C₅, and C₆ alkenyl groups. C₂₋₈ alkenyl is intended to include C₂, C₃, C₄, C₅, C₆, C₇, and C₈ alkenyl groups.

As used herein, “alkynyl” is intended to include hydrocarbon chains of either straight or branched configuration and one or more triple carbon-carbon bonds that may occur in any stable point along the chain, such as ethynyl and propynyl. C₂₋₆ alkynyl is intended to include C₂, C₃, C₄, C₅, and C₆ alkynyl groups. C₂₋₈ alkynyl is intended to include C₂, C₃, C₄, C₅, C₆, C₇, and C₈ alkynyl groups.

As used herein, “acyl” is intended to include hydrocarbon chains of either straight or branched configuration and one keto group (═O) that may occur in any stable point along the chain. “C₁₋₈ acyl” is intended to include C₂, C₃, C₄, C₅, C₆, C₇, and C₈ acyl groups.

As used herein, “alkoxy” refers to an alkyl group as defined above with the indicated number of carbon atoms attached through an oxygen bridge. C₁₋₆ alkoxy, is intended to include C₁, C₂, C₃, C₄, C₅, and C₆ alkoxy groups. C₁₋₈ alkoxy, is intended to include C₁, C₂, C₃, C₄, C₅, C₆, C₇, and C₈ alkoxy groups. Examples of alkoxy include, but are not limited to, methoxy, ethoxy, n-propoxy, i-propoxy, n-butoxy, s-butoxy, t-butoxy, n-pentoxy, s-pentoxy, n-heptoxy, and n-octoxy.

As used herein, “alkylthio” refers to an alkyl group as defined above with the indicated number of carbon atoms attached through an sulfur bridge. C₁₋₆ alkylthio, is intended to include C₁, C₂, C₃, C₄, C₅, and C₆ alkylthio groups. C₁₋₈ alkylthio, is intended to include C₁, C₂, C₃, C₄, C₅, C₆, C₇, and C₈ alkylthio groups.

As used herein, “carbocycle” or “carbocyclic ring” is intended to mean, unless otherwise specified, any stable 3, 4, 5, 6, or 7-membered monocyclic or bicyclic or 7, 8, 9, 10, 11, or 12-membered bicyclic or tricyclic ring, any of which may be saturated, unsaturated, or aromatic. Examples of such carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, [3.3.0]bicyclooctane, [4.3.0]bicyclononane, [4.4.0]bicyclodecane, [2.2.2]bicyclooctane, fluorenyl, phenyl, naphthyl, indanyl, adamantyl, and tetrahydronaphthyl. As shown above, bridged rings are also included in the definition of carbocycle (e.g., [2.2.2]bicyclooctane). A bridged ring occurs when one or more carbon atoms link two non-adjacent carbon atoms. Preferred bridges are one or two carbon atoms. It is noted that a bridge always converts a monocyclic ring into a tricyclic ring. When a ring is bridged, the substituents recited for the ring may also be present on the bridge. Fused (e.g., naphthyl and tetrahydronaphthyl) and spiro rings are also included.

As used herein, “halo” or “halogen” refers to fluoro, chloro, bromo, and iodo. “Counterion” is used to represent a small, negatively charged species such as chloride, bromide, hydroxide, acetate, and sulfate.

As used herein, the term “heterocycle” means, unless otherwise stated, a stable 3, 4, 5, 6, or 7-membered monocyclic or bicyclic or 7, 8, 9, 10, 11, or 12-membered bicyclic or tricyclic heterocyclic ring which is saturated, unsaturated, or aromatic, and consists of carbon atoms and one or more ring heteroatoms, e.g., 1 or 1-2 or 1-3 or 14 or 1-5 or 1-6 heteroatoms, independently selected from the group consisting of nitrogen, oxygen, and sulfur, and including any bicyclic group in which any of the above-defined heterocyclic rings is fused to a second ring (e.g., a benzene ring). The nitrogen and sulfur heteroatoms may optionally be oxidized (i.e., N→O and S(O)ₚ, where p=1 or 2). When a nitrogen atom is included in the ring it is either N or NH, depending on whether or not it is attached to a double bond in the ring (i.e., a hydrogen is present if needed to maintain the tri-valency of the nitrogen atom). The nitrogen atom may be substituted or unsubstituted (i.e., N or NR wherein R is H or another substituent, as defined). The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quaternized. It is preferred that when the total number of S and O atoms in the heterocycle exceeds 1, then these heteroatoms are not adjacent to one another. It is preferred that the total number of S and O atoms in the heterocycle is not more than 1. Bridged rings are also included in the definition of heterocycle. A bridged ring occurs when one or more atoms (i.e., C, O, N, or S) link two non-adjacent carbon or nitrogen atoms. Preferred bridges include, but are not limited to, one carbon atom, two carbon atoms, one nitrogen atom, two nitrogen atoms, and a carbon-nitrogen group. It is noted that a bridge always converts a monocyclic ring into a tricyclic ring. When a ring is bridged, the substituents recited for the ring may also be present on the bridge. Spiro and fused rings are also included.

As used herein, the term “heteroaryl” or “aromatic heterocycle” is intended to mean a stable 5, 6, or 7-membered monocyclic or bicyclic or 7, 8, 9, 10, 11, or 12-membered bicyclic heterocyclic aromatic ring which consists of carbon atoms and one or more heteroatoms, e.g., 1 or 1-2 or 1-3 or 1-4 or 1-5 or 1-6 heteroatoms, independently selected from the group consisting of nitrogen, oxygen, and sulfur. In the case of bicyclic heterocyclic aromatic rings, only one of the two rings needs to be aromatic (e.g., 2,3-dihydroindole), though both may be (e.g., quinoline). The second ring can also be fused or bridged as defined above for heterocycles. The nitrogen atom may be substituted or unsubstituted (i.e., N or NR wherein R is H or another substituent, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1.

Examples of heterocycles include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, dihydrooxazole, dithiazolonyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isopyrrolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, 1,2,3-oxathiazolyl-1-oxide, oxathiolyl, oxazolidinyl, oxazolyl, oxindolyl, oxo-imidazolyl, oxo-thiazolinyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetraydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiatriazolyl, thiazoledionyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

The term “hydroxy protecting group” refers to a selectively removable group which is known in the art to protect a hydroxyl group against undesirable reaction during synthetic procedures. The use of hydroxy-protecting groups is well known in the art and many such protecting groups are known (see, for example, T. H. Greene and P. G. M. Wuts (1999) PROTECTIVE GROUPS IN ORGANIC SYNTHESIS, 3rd edition, John Wiley & Sons, New York). Examples of hydroxy protecting groups include, but are not limited to, acetate, methoxymethyl ether, methylthiomethyl, tert-butyldimethylsilyl, and tert-butyldiphenylsilyl.

The term “macrolide” refers to any compound possessing a 14- or 15-membered macrocyclic ring and derivatives thereof (such as keto, oxime, cyclic carbonate derivatives). These include, for example, compounds that are (or are synthetically derived from) known antibacterial agents including, but not limited to, erythromycin, clarithromycin, azithromycin, telithromycin, roxithromycin, pikromycin, flurithromycin, and dirithromycin.

As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, and toluene sulfonic.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, 1445.

The term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Other suitable ester groups include, for example, those derived from pharmaceutically acceptable alcohols, such as straight-chain or branched aliphatic alcohols, benzylic alcohols, and amino-alcohols. Examples of particular esters include formates, acetates, propionates, butyrates, acrylates, ethylsuccinates, and methyl, ethyl, propyl, benzyl, and 2-aminoethyl alcohol esters.

Since prodrugs are known to enhance numerous desirable qualities of pharmaceuticals (e.g., solubility, bioavailability, manufacturing, etc.) the compounds of the present invention may be delivered in prodrug form. Thus, the present invention is intended to cover prodrugs of the presently claimed compounds, methods of delivering the same and compositions containing the same. “Prodrugs” are intended to include any covalently bonded carriers that release an active parent drug of the present invention in vivo when such prodrug is administered to a mammalian subject. Prodrugs the present invention are prepared by modifying functional groups present in the compound in such a way that the modifications are cleaved, either in routine manipulation or in vivo, to the parent compound. Prodrugs include compounds of the present invention wherein a hydroxy, amino, or sulfhydryl group is bonded to any group that, when the prodrug of the present invention is administered to a mammalian subject, it cleaves to form a free hydroxyl, free amino, or free sulfhydryl group, respectively. Examples of prodrugs include, but are not limited to, acetate, formate, and benzoate derivatives of alcohol and amine functional groups in the compounds of the present invention.

“Stable compound” and “stable structure” are meant to indicate a compound that is sufficiently robust to survive isolation to a useful degree of purity from a reaction mixture, and formulation into an efficacious therapeutic agent. It is preferred that the presently recited compounds do not contain a N-halo, S(O)₂H, or S(O)H group.

As used herein, “treating” or “treatment” means the treatment of a disease-state in a mammal fish, or fowl, particularly in a human, and include: (a) preventing the disease-state from occurring in a mammal fish, or fowl, in particular, when such mammal fish, or fowl is predisposed to the disease-state but has not yet been diagnosed as having it; (b) inhibiting the disease-state, i.e., arresting its development; and/or (c) relieving the disease-state, i.e., causing regression of the disease state.

As used herein, “mammal” refers to human and non-human patients.

As used herein, the term “therapeutically effective amount” refers to an amount of a compound, or a combination of compounds, of the present invention effective when administered alone or in combination as an anti-proliferative and/or anti-infective agent. The combination of compounds is preferably a synergistic combination. Synergy, as described, for example, by Chou and Talalay, Adv. Enzyme Regul. 1984, 22:27-55, occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, increased anti-proliferative and/or anti-infective effect, or some other beneficial effect of the combination compared with the individual components.

All percentages and ratios used herein, unless otherwise indicated, are by weight.

Throughout the description, where compositions are described as having, including, or comprising specific components, or where processes are described as having, including, or comprising specific process steps, it is contemplated that compositions of the present invention also consist essentially of, or consist of, the recited components, and that the processes of the present invention also consist essentially of, or consist of, the recited processing steps. Further, it should be understood that the order of steps or order for performing certain actions are immaterial so long as the invention remains operable. Moreover, two or more steps or actions may be conducted simultaneously.

### 2. COMPOUNDS OF THE INVENTION

The invention provides a compound having the formula: 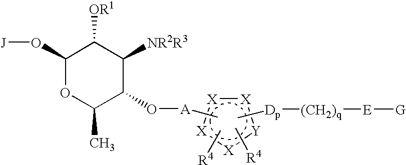
or a pharmaceutically acceptable salt, ester, or prodrug thereof,

wherein:

—O-A is selected from the group consisting of: 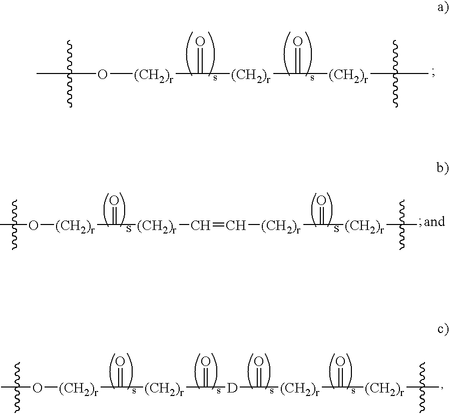
wherein
   r, at each occurrence, independently is 0, 1, 2 3, or 4, and
   s, at each occurrence, independently is 0 or 1;

X, at each occurrence, independently is carbon, carbonyl, or nitrogen, provided at least one X is carbon;

Y is carbon, nitrogen, oxygen, or sulfur;

D is selected from the group consisting of:
O, S, NR⁵, C═O, C═S, C═NOR⁵, SO, and SO₂;

E-G is selected from the group consisting of

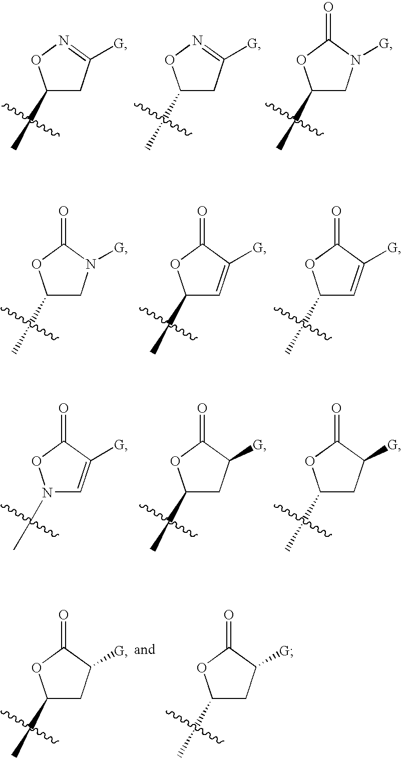

G is selected from the group consisting of: 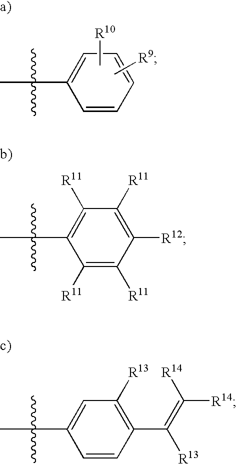
d) 3-14 membered saturated, unsaturated, or aromatic heterocycle containing one or more heteroatoms selected from the group consisting of nitrogen, oxygen, and sulfur, and optionally substituted with one or more 10 groups;
e) C₃₋₁₄ saturated, unsaturated, or aromatic carbocycle, optionally substituted with one or more R⁴ groups;
f) C₁₋₈ alkyl,
g) C₂₋₈ alkenyl,
h) C₂₋₈ alkynyl,
i) C₁₋₈ alkoxy,
j) C₁₋₈ alkylthio,
k) C₁₋₈ acyl,
l) S(O)ₜR⁵; and
m) hydrogen,
wherein any of f)-k) optionally is substituted with
   i) one or more R⁴ groups;
   ii) 3-14 membered saturated, unsaturated, or aromatic heterocycle containing one or more heteroatoms selected from the group consisting of nitrogen, oxygen, and sulfur, and optionally substituted with one or more R⁴ groups; or
   iii) C₃₋₁₄ saturated, unsaturated, or aromatic carbocycle, optionally substituted with one or more R⁴ groups;

J is selected from the group consisting of:
a) H, b) Lᵤ-C₁₋₆ alkyl, c) Lᵤ-C₂₋₆ alkenyl, d) Lᵤ-C₂₋₆ alkynyl, e) Lᵤ-C₃₋₁₄ saturated, unsaturated, or aromatic carbocycle, f) Lᵤ-(3-14 membered saturated, unsaturated, or aromatic heterocycle comprising one or more heteroatoms selected from the group consisting of nitrogen, oxygen, and sulfur), and g) macrolide,
   wherein
      L is selected from the group consisting of —C(O)—, —C(O)O—, and —C(O)NR⁵—,
      u is 0 or 1, and
      any of b)-f) optionally is substituted with one or more R⁴ groups;

R¹, R², and R³ are independently selected from the group consisting of:
a) H, b) Lᵤ-C₁₋₆ alkyl, c) Lᵤ-C₂₋₆ alkenyl, d) Lᵤ-C₂₋₆ alkynyl, e) Lᵤ-C₃₋₁₄ saturated, unsaturated, or aromatic carbocycle, f) Lᵤ-(3-14 membered saturated, unsaturated, or aromatic heterocycle comprising one or more heteroatoms selected from the group consisting of nitrogen, oxygen, and sulfur), g) Lᵤ-(saturated, unsaturated, or aromatic 10-membered bicyclic ring system optionally containing one or more heteroatoms selected from the group consisting of nitrogen, oxygen, and sulfur), and h) Lᵤ-(saturated, unsaturated, or aromatic 13-membered tricyclic ring system optionally containing one or more heteroatoms selected from the group consisting of nitrogen, oxygen, and sulfur),
   wherein
      L is selected from the group consisting of —C(O)—, —C(O)O—, and —C(O)NR⁷—,
      u is 0 or 1, and
      any of b)-h) optionally is substituted with one or more R⁴ groups;

alternatively, R², and R³, taken together with the nitrogen atom to which they are bonded, form a 5-7 membered saturated, unsaturated, or aromatic heterocycle optionally containing one or more additional atoms selected from the group consisting of nitrogen, oxygen, and sulfur, and optionally substituted with one or more R⁴ groups;

R⁴, at each occurrence, independently is selected from the group consisting of:
a) F, b) Cl, c) Br, d) I, e) ═O, f) ═S, g) ═NR⁵, h) ═NOR⁵, i) ═NS(O)ₜR⁵, j) ═N—NR⁵R⁵, k) —CF₃, l) —OR⁵, m) —CN, n) —NO₂, o) —NR⁵R⁵, p) —NR⁵OR⁵, q) —C(O)R⁵, r) —C(O)OR⁵, s) —OC(O)R⁵, t) —C(O)NR⁵R⁵, u) —NR⁵C(O)R⁵, v) —OC(O)NR⁵R⁵, w) —NR⁵C(O)OR⁵, x) —NR⁵C(O)NR⁵R⁵, y) —C(S)R⁵, z) —C(S)OR⁵, aa) —OC(S)R⁵, bb) —C(S)NR⁵R⁵, cc) —NR⁵C(S)R⁵, dd) —OC(S)NR⁵R⁵, ee) —NR⁵C(S)OR⁵, ff) —NR⁵C(S)NR⁵R⁵, gg) —C(═NR⁵)R⁵; hh) —C(═NR⁵)OR⁵, ii) —OC(═NR⁵)R⁵, jj) —C(═NR⁵)NR⁵R⁵, kk) —NR⁵C(═NR⁵)R⁵, ll) —OC(—NR⁵)NR⁵R, mm) —NR⁵C(═NR⁵)OR⁵, nn) —NR⁵C(═NR⁵)NR⁵R⁵, oo) —NR⁵C(═NR⁵)NR⁵R⁵, pp) —S(O)ₜR⁵, qq) —SO₂NR⁵R⁵, rr) —S(O)ₜN═R⁵, and ss) R⁵;

R⁵, at each occurrence, independently is selected from the group consisting of:
a) H, b) Lᵤ-C₁₋₆ alkyl, c) Lᵤ-C₂₋₆ alkenyl, d) Lᵤ-C₂₋₆ alkynyl, e) Lᵤ-C₃₋₁₄ saturated, unsaturated, or aromatic carbocycle, f) Lᵤ-(3-14 membered saturated, unsaturated, or aromatic heterocycle comprising one or more heteroatoms selected from the group consisting of nitrogen, oxygen, and sulfur), g) Lᵤ-(saturated, unsaturated, or aromatic 10-membered bicyclic ring system optionally containing one or more heteroatoms selected from the group consisting of nitrogen, oxygen, and sulfur), and h) Lᵤ-(saturated, unsaturated, or aromatic 13-membered tricyclic ring system optionally containing one or more heteroatoms selected from the group consisting of nitrogen, oxygen, and sulfur),
   wherein
      L is selected from the group consisting of —C(O)—, —C(O)O—, and —C(O)NR⁸—,
      u is 0 or 1, and
      any of b)-h) optionally is substituted with one or more R⁶ groups;

alternatively, two R⁵ groups, taken together with the atom or atoms to which they are bonded, form i) a 5-7 membered saturated, unsaturated, or aromatic carbocycle, or ii) a 5-7 membered saturated, unsaturated, or aromatic heterocycle containing one or more atoms selected from the group consisting of nitrogen, oxygen, and sulfur,
wherein i)-ii) optionally is substituted with one or more R⁶ groups;

R⁶, at each occurrence, independently is selected from the group consisting of:
a) F, b) Cl, c) Br, d) 1, e) —O, f) ═S, g) ═NR⁷, h) ═NOR⁷, i) —NS(O)ₜR⁷, j) —N—NR⁷R⁷, k) —CF₃, l) —OR⁷, m) —CN, n) —NO₂, o) —NR⁷R⁷, p) —NR⁷OR⁷, q) —C(O)R⁷, r) —C(O)OR⁷, s) —OC(O)R⁷, t) —C(O)NR⁷R⁷, u) —NR⁷C(O)R⁷, v) —OC(O)NR⁷R⁷, w) —NR⁷C(O)OR⁷, x) —NR⁷C(O)NR⁷R⁷, y) —C(S)R⁷, z) —C(S)OR⁷, aa) —OC(S)R⁷, bb) —C(S)NR⁷R⁷, cc) —NR⁷C(S)R⁷, dd) —OC(S)NR⁷R⁷, ee) —NR⁷C(S)OR⁷, ff) —NR⁷C(S)NR⁷R⁷, gg) —C(—NR⁷)R⁷; hh) —C(═NR⁷)OR⁷, ii) —OC(—NR⁷)R⁷, jj) —C(—NR⁷)NR⁷R⁷, kk) —NR⁷C(═NR⁷)R⁷, ll) —OC(═NR⁷)NR⁷R⁷, mm) —NR⁷C(—NR⁷)OR⁷, nn) —NR⁷C(═NR⁷)NR⁷R⁷, oo) —NR⁷C(═NR⁷)NR⁷R⁷, pp) —S(O)ₜR⁷, qq) —SO₂NR⁷R⁷, rr) —S(O)ₜN═R⁷, and ss) R⁷;

R⁷, at each occurrence, independently is selected from the group consisting of:
a) H, b) Lᵤ-C₁₋₆ alkyl, c) Lᵤ-C₂₋₆ alkenyl, d) Lᵤ-C₂₋₆ alkynyl, e) Lᵤ-C₃₋₁₄ saturated, unsaturated, or aromatic carbocycle, f) Lᵤ-(3-14 membered saturated, unsaturated, or aromatic heterocycle comprising one or more heteroatoms selected from the group consisting of nitrogen, oxygen, and sulfur), g) Lᵤ-(saturated, unsaturated, or aromatic 10-membered bicyclic ring system optionally containing one or more heteroatoms selected from the group consisting of nitrogen, oxygen, and sulfur), and h) Lᵤ-(saturated, unsaturated, or aromatic 13-membered tricyclic ring system optionally containing one or more heteroatoms selected from the group consisting of nitrogen, oxygen, and sulfur),
   wherein
      L is selected from the group consisting of C(O), C(O)O, and C(O)NR⁷,
      u is 0 or 1, and
      any of b)-h) optionally is substituted with one or more moieties selected from the group consisting of:
         R⁸, F, Cl, Br, I, —CF₃, —OR⁸, —SR⁸, —CN, —NO₂, —NR⁸R⁸, —C(O)R⁸, —C(O)OR⁸, —OC(O)R⁸, —C(O)NR⁸R⁸, —NR⁸C(O)R⁸, —OC(O)NR⁸R⁸, —NR⁸C(O)OR⁸, —NR⁸C(O)NR⁸R⁸, —C(S)R⁸, —C(S)OR⁸, —OC(S)R⁸, —C(S)NR⁸R⁸, —NR⁸C(S)R⁸, —OC(S)NR⁸R⁸, —NR⁸C(S)OR⁵, —NR⁸C(S)NR⁸R⁸, —NR⁸C(NR⁸)NR⁸R⁸, —SO₂NR⁸R⁸, and —S(O)ₜR⁸;

alternatively, two R⁷ groups, taken together with the atom or atoms to which they are bonded, form i) a 5-7 membered saturated, unsaturated, or aromatic carbocycle, or ii) a 5-7 membered saturated, unsaturated, or aromatic heterocycle containing one or more atoms selected from the group consisting of nitrogen, oxygen, and sulfur;

R⁸, at each occurrence, independently is selected from the group consisting of:
a) H, b) Lᵤ-C₁₋₈ alkyl, c) Lᵤ-C₂₋₆ alkenyl, d) Lᵤ-C₂₋₆ alkynyl, e) Lᵤ-C₃₋₁₄ saturated, unsaturated, or aromatic carbocycle, f) Lᵤ-(3-14 membered saturated, unsaturated, or aromatic heterocycle comprising one or more heteroatoms selected from the group consisting of nitrogen, oxygen, and sulfur), g) Lᵤ-(saturated, unsaturated, or aromatic 10-membered bicyclic ring system optionally containing one or more heteroatoms selected from the group consisting of nitrogen, oxygen, and sulfur), and h) Lᵤ-(saturated, unsaturated, or aromatic 13-membered tricyclic ring system optionally containing one or more heteroatoms selected from the group consisting of nitrogen, oxygen, and sulfur),
   wherein
      L is selected from the group consisting of —C(O)—, —C(O)O—, and —C(O)NH—, —C(O)N(C₁₋₆ alkyl)-and
      u is 0 or 1;

R⁹ is R⁴;

R¹⁰ is R⁴;

alternatively, R⁹ and R¹⁰, taken together with the atoms to which they are bonded, form i) a 5-7 membered saturated, unsaturated, or aromatic carbocycle, or ii) a 5-7 membered saturated, unsaturated, or aromatic heterocycle containing one or more atoms selected from the group consisting of nitrogen, oxygen, and sulfur,
wherein i)-ii) optionally is substituted with one or more R⁴ groups;

R¹¹ is R⁴;

alternatively, two R¹¹ groups, taken together with the atoms to which they are bonded, form i) a 5-7 membered saturated, unsaturated, or aromatic carbocycle, or ii) a 5-7 membered saturated, unsaturated, or aromatic heterocycle containing one or more atoms selected from the group consisting of nitrogen, oxygen, and sulfur,
wherein i)-ii) optionally is substituted with one or more R⁴ groups;

R¹² is R⁵;

alternatively, R¹² and one R¹¹ group, taken together with the atoms to which they are bonded, form i) a 5-7 membered saturated, unsaturated, or aromatic carbocycle, or ii) a 5-7 membered saturated, unsaturated, or aromatic heterocycle containing one or more atoms selected from the group consisting of nitrogen, oxygen, and sulfur,
wherein i)-ii) optionally is substituted with one or more R⁴ groups;

R¹³ is R⁴;

R¹⁴ is R⁴;

alternatively, any R¹³ and any R¹⁴, taken together with the atoms to which they are bonded, form i) a 5-7 membered saturated, unsaturated, or aromatic carbocycle, or ii) a 5-7 membered saturated, unsaturated, or aromatic heterocycle containing one or more atoms selected from the group consisting of nitrogen, oxygen, and sulfur,
wherein i)-ii) optionally is substituted with one or more R⁴ groups;

p is 0 or 1;

q is 0 or 1; and

t, at each occurrence, independently is 0, 1, or 2.

In certain embodiments, the invention provides compounds having the formula: 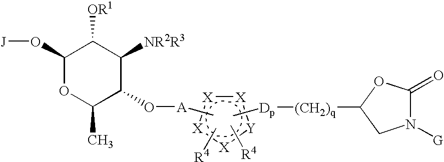
wherein A, D, G, J, R¹, R², R³, R⁴, X, Y, p, and q are as defined above.

In other embodiments, the invention provides compounds having the formula: 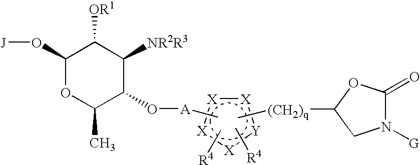
wherein O-A is O—(CH₂)ᵣ, O—C(O), or O—C(O)—(CH₂)ᵣ; r is 1, 2, 3, or 4; J is a macrolide; and G, R¹, R², R³, R⁴, X, Y, and q are as defined above.

In still other embodiments, the invention provides compounds having the formula:

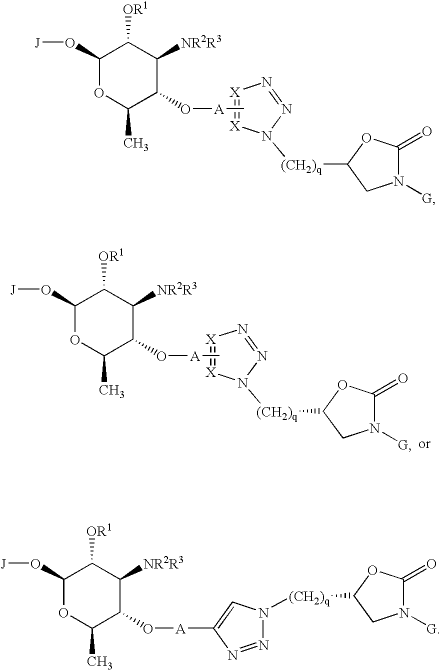

In certain embodiments of the foregoing compounds, G has the formula: 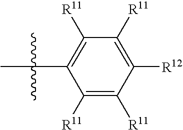
wherein R¹¹ and R¹² are as previously defined. In particular embodiments of these compounds, R¹² is —C(O)CH₃. In other embodiments, R¹² has the formula: 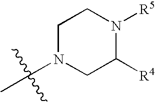
wherein R⁴ and R⁵ are as defined above. In certain embodiments of these compounds, R⁵ is —C(O)—CH₂—OH. In other embodiments, R⁴ is H.

In other embodiments, G has the formula: 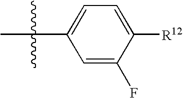
wherein R¹² is as described above. In certain embodiments of these compounds, R¹² is H. In other embodiments, R¹² has the formula: 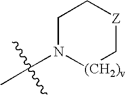
wherein Z is selected from the group consisting of O, NR⁵, and S(O)ₜ; and v is 0, 1, 2, or 3. In particular embodiments, Z is O and v is 1.

In certain embodiments, the invention provides compounds having the formula: 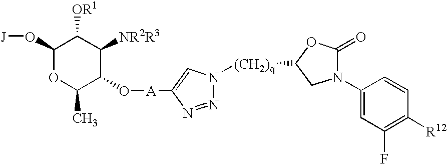
wherein O-A is O—(CH₂)ᵣ, O—C(O), or O—C(O)—(CH₂)ᵣ; r is 1, 2, 3, or 4; J is a macrolide; and R¹, R², R³, R¹², and q are as defined above. In embodiments of these compounds, R¹² is H or

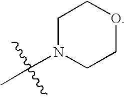

In still other embodiments of the foregoing compounds, J is a macrolide. In certain embodiments of these compounds, the macrolide is selected from the group consisting of: 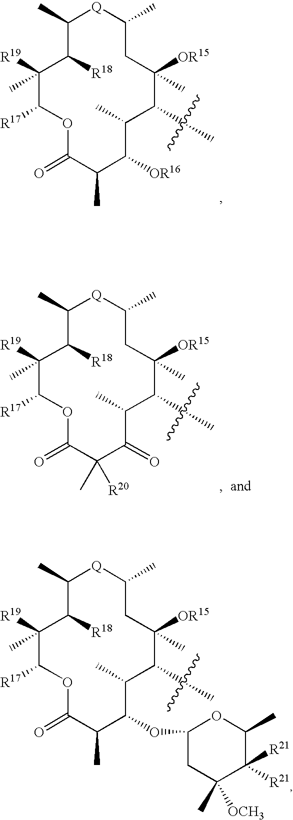
and pharmaceutically acceptable salts, esters and prodrugs thereof, wherein

Q is selected from the group consisting of:
—NR⁵CH₂, —CH₂—NR⁵—, —C(O)—, —C(═NR⁵)—, —C(═NOR⁵)—, —C(═N—NR⁵R⁵)—, —CH(OR⁵)—, and —CH(NR⁵R⁵)—;

R¹⁵ and R¹⁶ independently are selected from the group consisting of R⁵ and a hydroxy protecting group;

alternatively R¹⁵ and R¹⁶, taken together with the atoms to which they are bonded, form:

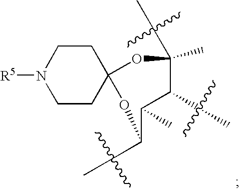

R¹⁷ is selected from the group consisting of:
a) C₁₋₆ alkyl, b) C₂₋₆ alkenyl, and c) C₂₋₆ alkynyl;
   wherein any of a)-c) optionally is substituted with one or more moieties selected from the group consisting of
      i) —OR⁵, ii) C₃₋₁₄ saturated, unsaturated, or aromatic carbocycle, and iii) 3-14 membered saturated, unsaturated, or aromatic heterocycle containing one or more atoms selected from the group consisting of nitrogen, oxygen, and sulfur,
         wherein any of ii)-iii) optionally is substituted with one or more R⁴ groups;

R¹⁸ is selected from the group consisting of:
a) OR¹⁵, b) C₁₋₆ alkyl, c) C₂₋₆ alkenyl, d) C₂₋₆ alkynyl, e) —C(O)R⁵, and f) —NR⁵R⁵,
   wherein any of b)-d) optionally is substituted with one or more R⁴ groups;

alternatively, R¹⁵ and R¹⁸, taken together with the atoms to which they are bonded, form: 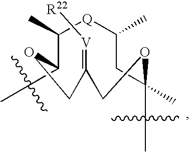
wherein
   V is CH or N, and
   R²² is —OR⁵, or R⁵;

R¹⁹ is —OR¹⁵;

alternatively, R¹⁸ and R¹⁹, taken together with the atoms to which they are bonded, form a 5-membered ring by attachment to each other through a linker selected from the group consisting of:
—OC(R⁴)(R⁴)O—, —OC(O)O—, —OC(O)NR⁵—, —NR⁵C(O)O—, —OC(O)NOR⁵—, —N(OR⁵)C(O)O—, —OC(O)N—NR⁵R⁵—, —N(NR⁵R⁵)C(O)O—, —OC(O)CHR⁵—, —CHR⁴C(O)O—, —OC(S)O—, —OC(S)NR⁵—, —NR⁵C(S)O—, —OC(S)NOR⁵—, —N(OR⁵)C(S)O, —OC(S)N—NR⁵R⁵—, —N(NR⁵R⁵)C(S)O, —OC(S)CHR⁴—, and CHR⁴C(S)O—;

alternatively, Q, R¹⁸, and R¹⁹, taken together with the atoms to which they are bonded, form: 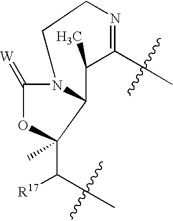
wherein
   W is O, NR⁵, or NOR⁵;

R²⁰ is selected from the group consisting of:
H, F, Cl, Br, and C₁₋₆ alkyl;

R²¹, at each occurrence, independently is selected from the group consisting of:
R⁵, —OR¹⁵, and —NR⁵R⁵;

alternatively, two R²¹ groups taken together are ═O, ═N—OR⁵, or ═N—NR⁵R⁵.

In particular embodiments, J is selected from the group consisting of:

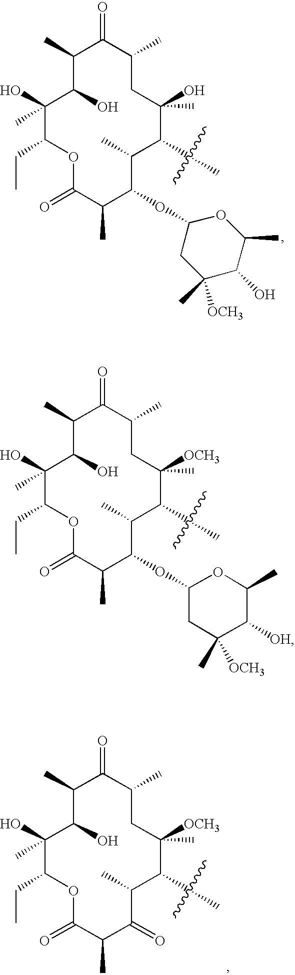

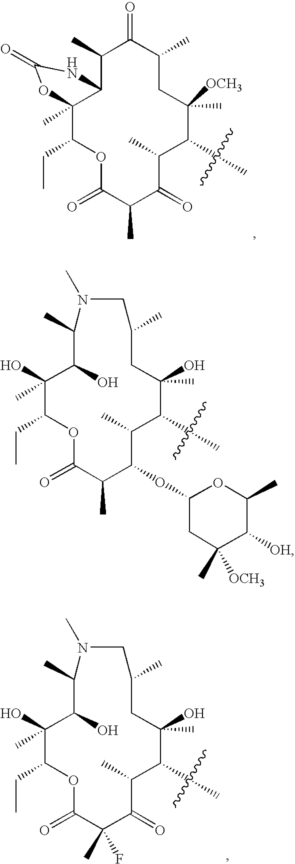

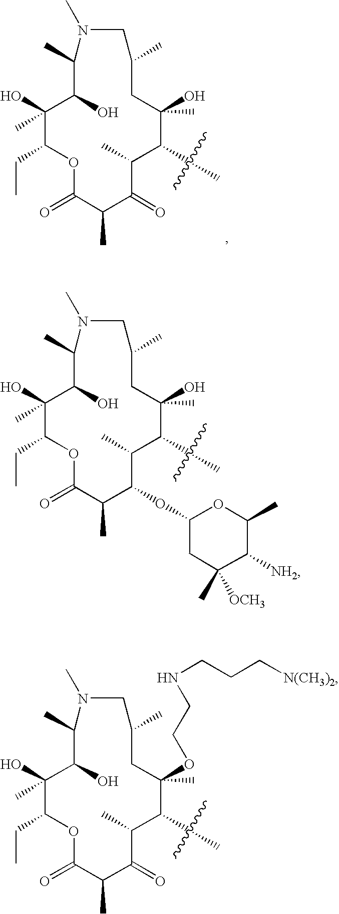

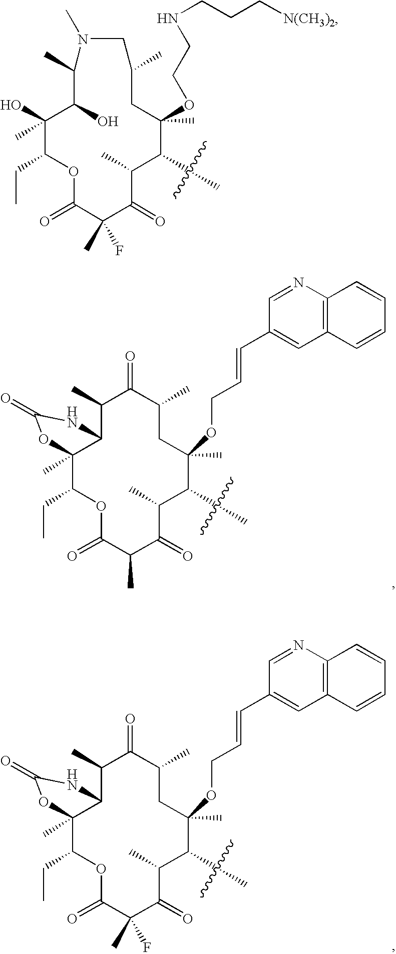

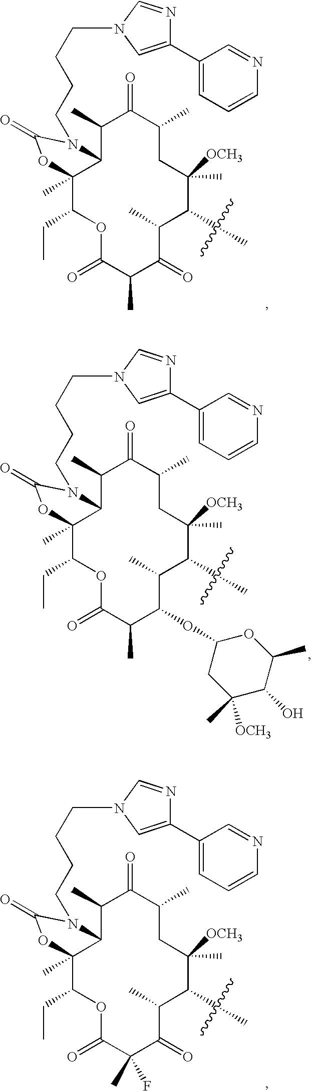

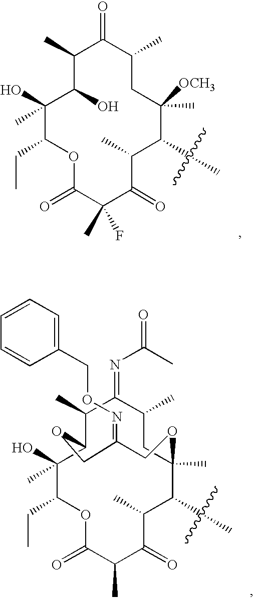

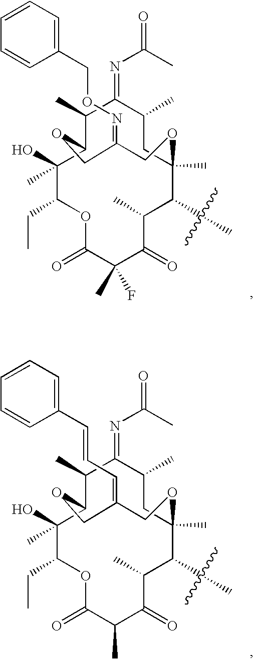

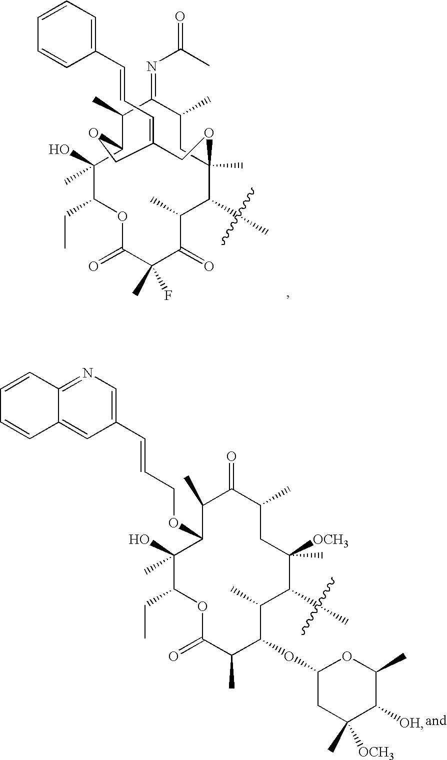

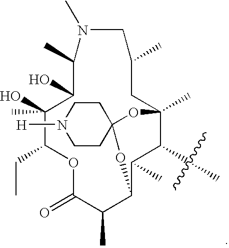

In other embodiments of the foregoing compounds, R¹ is H; R² is methyl, and R³ is methyl.

Particular embodiments of the invention include: 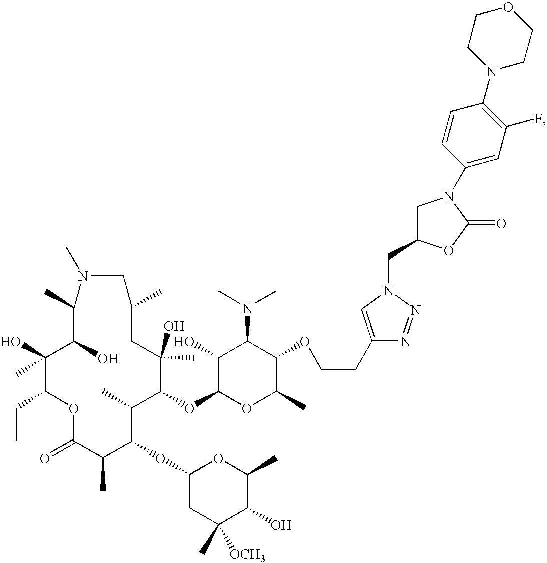 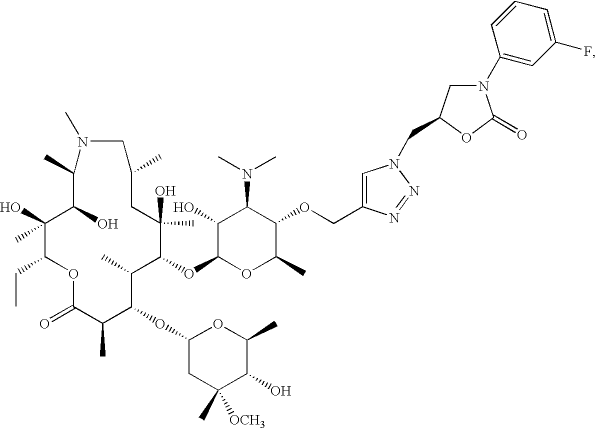 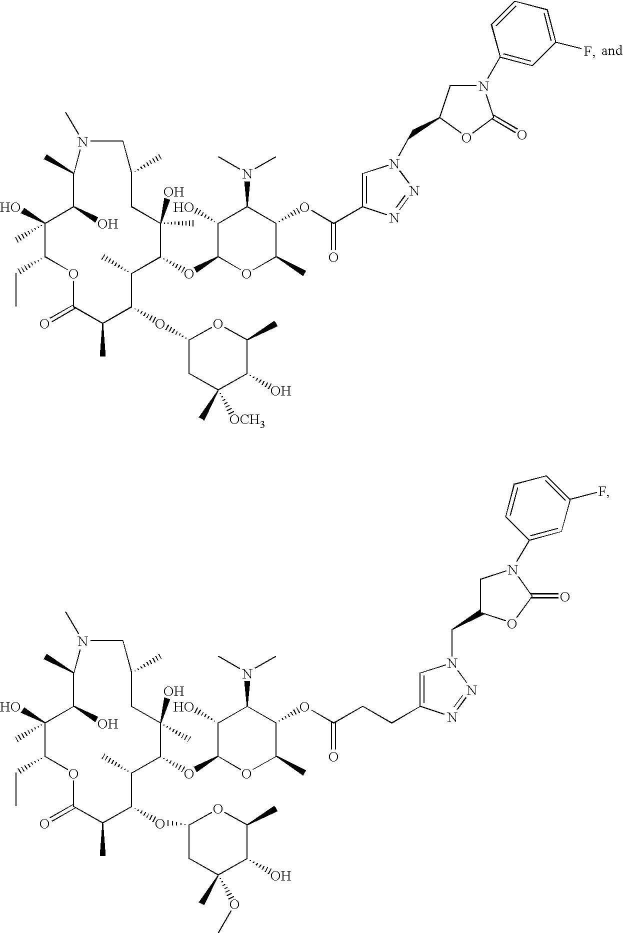
or a pharmaceutically acceptable salt, ester, or prodrug thereof.

In another aspect, the invention provides a pharmaceutical composition comprising a therapeutically effective amount of one or more of the foregoing compounds and a pharmaceutically acceptable carrier. In yet another aspect, the invention provides a method for treating a microbial infection, a fungal infection, a viral infection, a parasitic disease, a proliferative disease, an inflammatory disease, or a gastrointestinal motility disorder in a mammal, fish, or fowl by administering effective amounts of the compounds of the invention or pharmaceutical compositions of the invention, for example, via oral, parenteral or topical routes. In still another aspect, the invention provides methods for synthesizing any one of the foregoing compounds. In another aspect, the invention provides a medical device, for example, a medical stent, which contains or is coated with one or more of the foregoing compounds.

In another embodiment, the invention further provides a family of compounds comprising a heterocyclic side-chain linked via a heterocyclic linker to at least a portion of a macrolide. Exemplary macrolides, heterocyclic linkers, and heterocyclic side-chains useful in the synthesis of the compounds include, but are not limited to, the chemical moieties shown below:
Macrolides

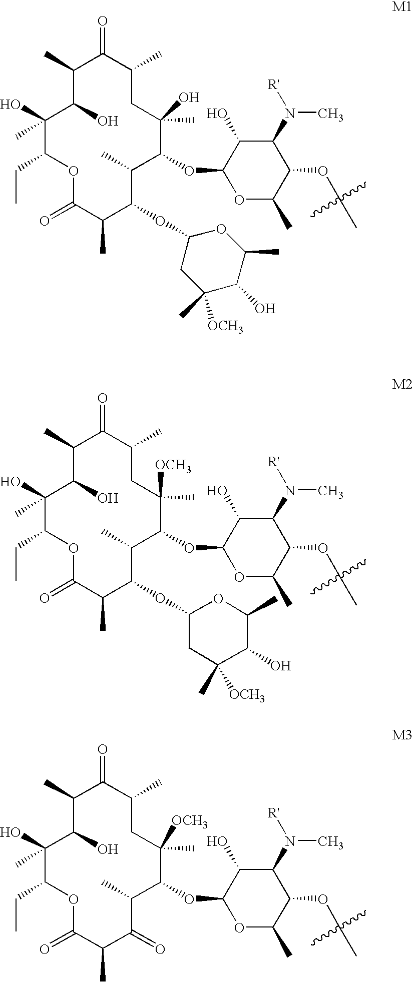

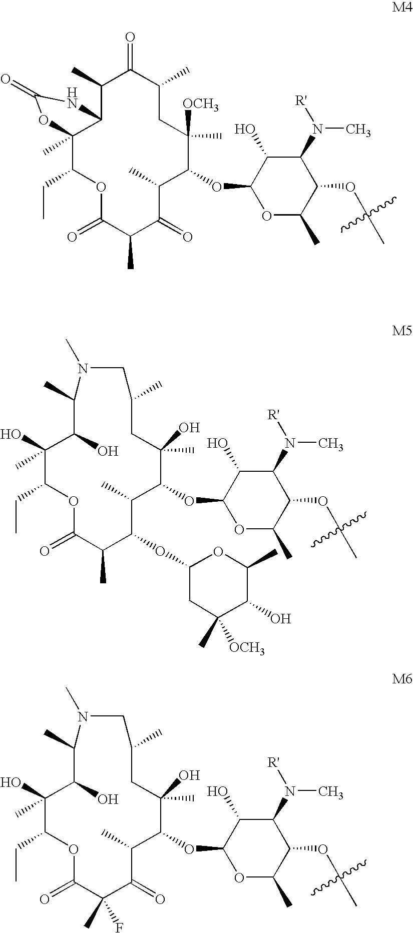

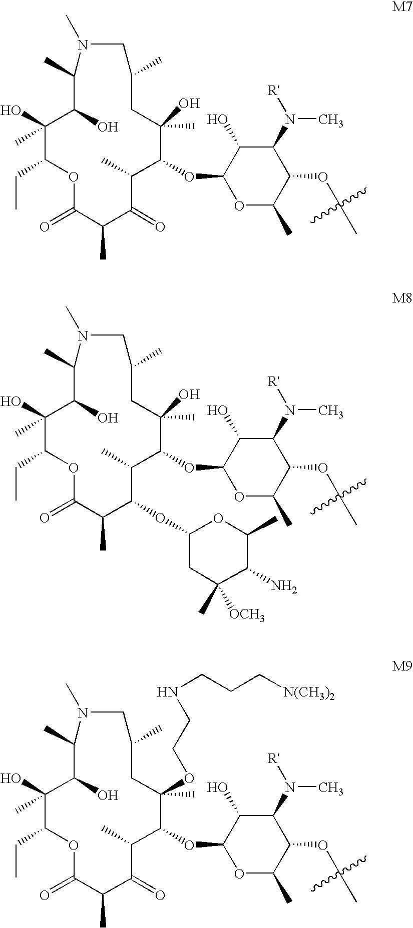

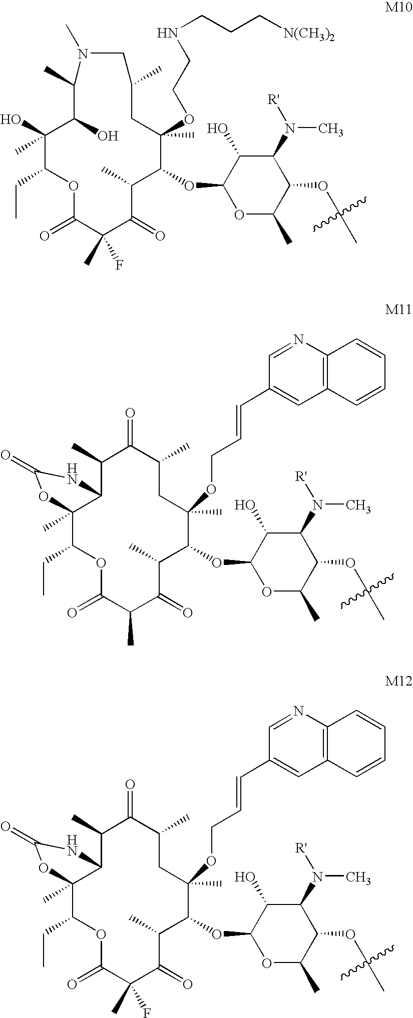

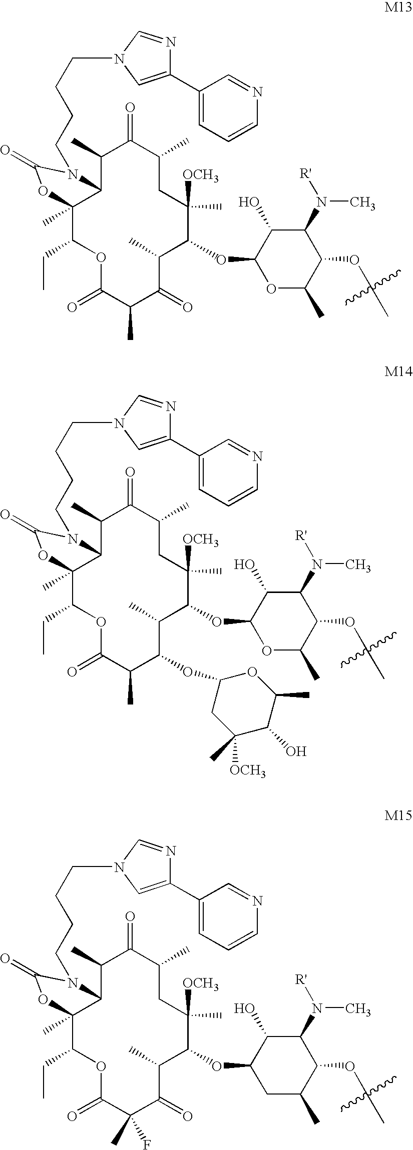

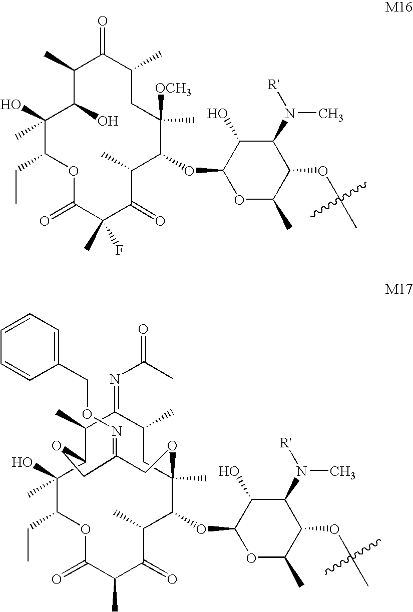

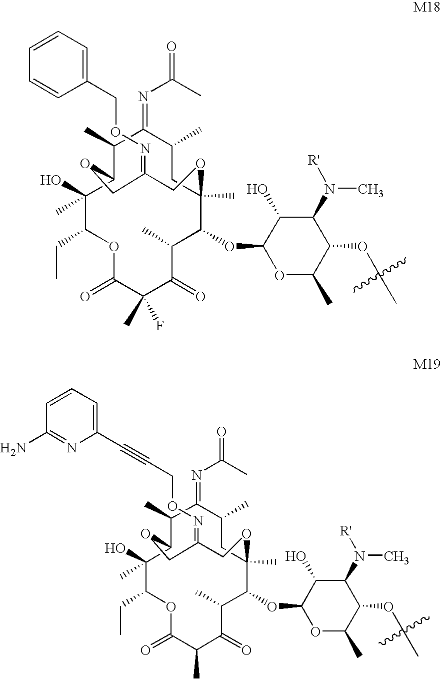

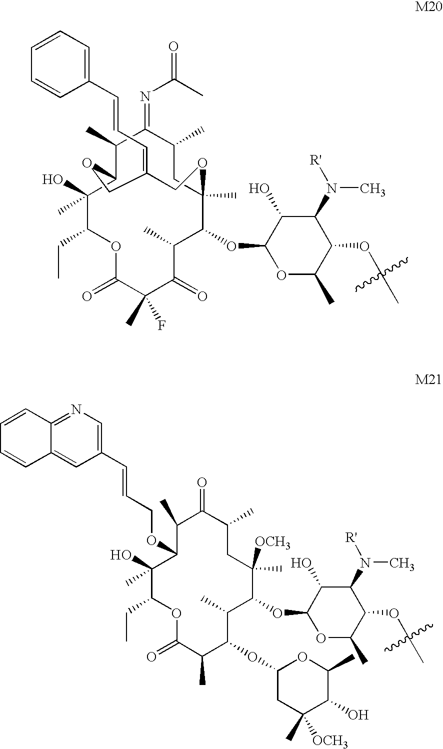

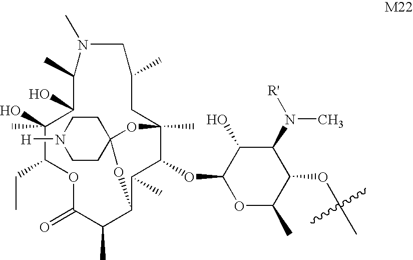

For the above macrolides, R′ can be either hydrogen or methyl.
Linkers

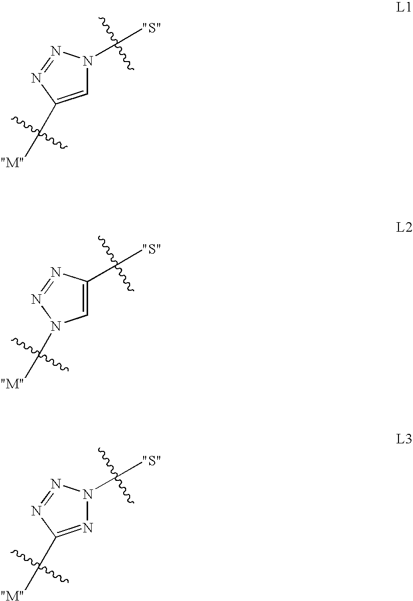

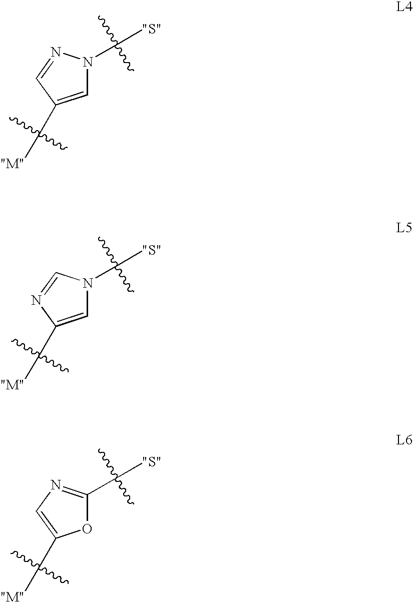

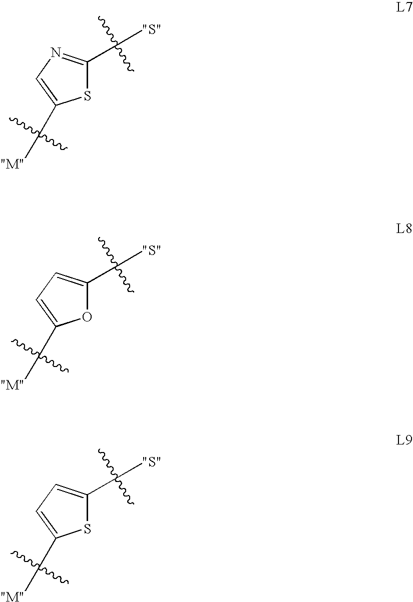

For the above heterocyclic linkers, “M” and “S” are included to depict the orientation of the heterocyclic linker with respect to the other structures that define the compounds of the invention. More specifically, “M” denotes the portion of the compound that includes the macrolide moiety, and “S” denotes the portion of the compound that includes the heterocyclic side-chain moiety.
Side-Chains

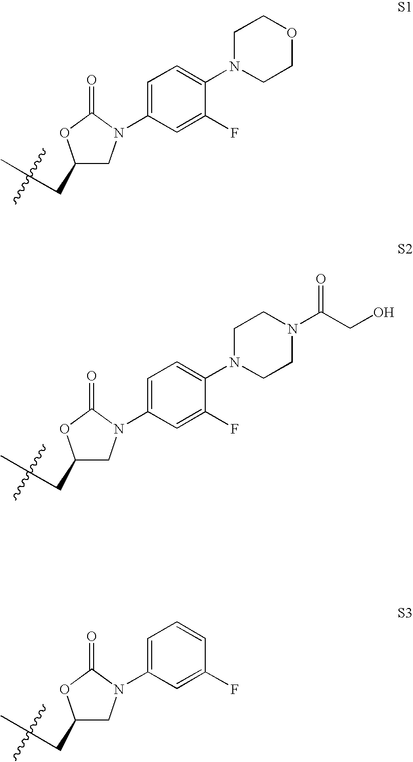

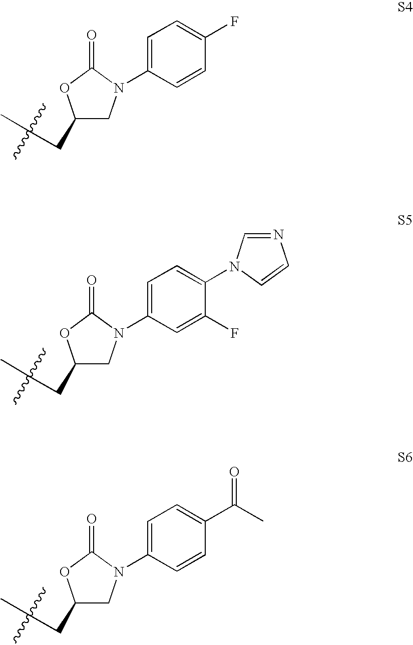

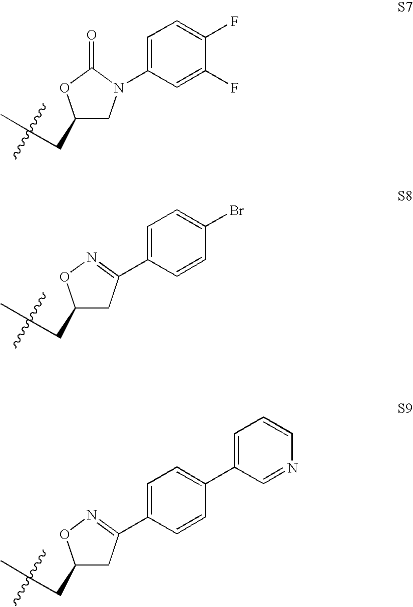

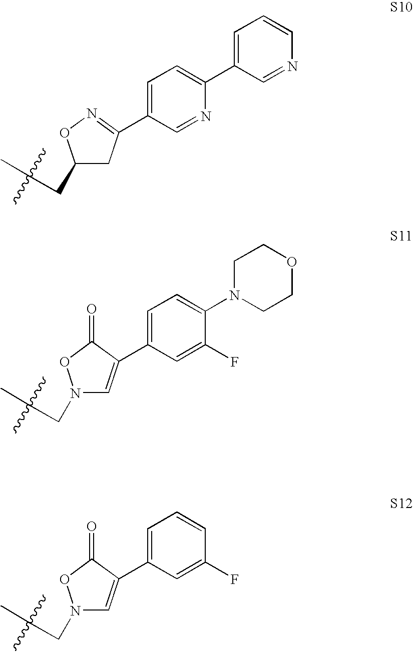

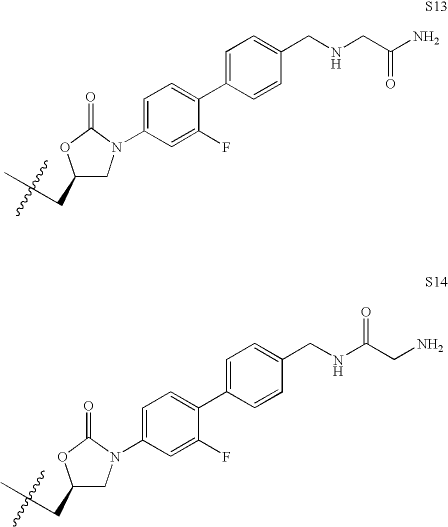

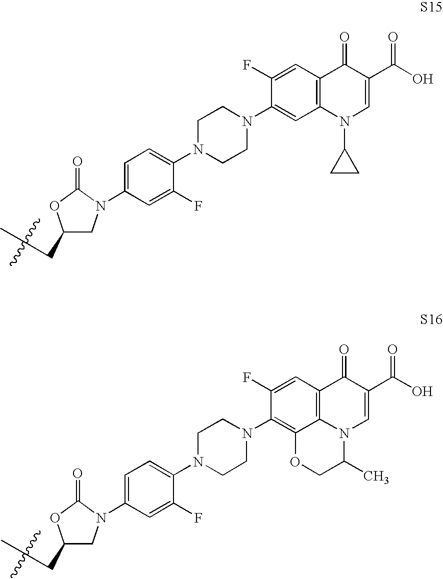

An exemplary scheme showing the linkage of a heterocyclic side-chain to a macrolide fragment via a heterocyclic linker is depicted below, where R′ is hydrogen or methyl and n is 1, 2, 3, or 4:

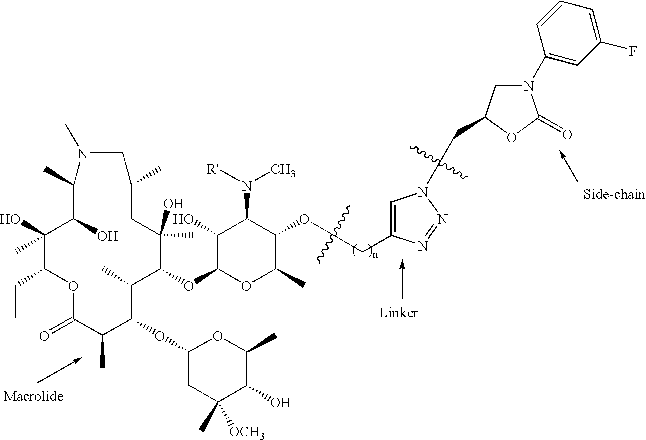

The various heterocyclic side-chains may be linked via the heterocyclic linkers to the macrolides using conventional chemistries known in the art, such as those discussed below. By using the various combinations of chemical moieties provided, the skilled artisan may synthesize one or more of the exemplary compounds listed below in Table 2. For each set of examples, the lower case letter designations denote compounds where R′ is hydrogen or methyl and n is 1, 2, 3, or 4. The R′ and n values for each lower case letter designation are set forth in Table 1 below.

TABLE 1

| Compound | R′ | n |
|---|---|---|
| a | H | 1 |
| b | H | 2 |
| c | H | 3 |
| d | H | 4 |
| e | methyl | 1 |
| f | methyl | 2 |
| g | methyl | 3 |
| h | methyl | 4 |

For example, as a guide to Table 2, compound E1a is the R′=H, n=1 variant of the structure shown on the row 1 of the table, compound E1b is the R′=H, n=2 derivative, and E1e is the R′=methyl, n=1 derivative.

TABLE 2

| Example | S Group | L Group | M Group |
|---|---|---|---|
| E1a-h | S1 | L1 | M1 |
| E2a-h | S1 | L2 | M1 |
| E3a-h | S1 | L3 | M1 |
| E4a-h | S1 | L4 | M1 |
| E5a-h | S1 | L5 | M1 |
| E6a-h | S1 | L6 | M1 |
| E7a-h | S1 | L7 | M1 |
| E8a-h | S1 | L8 | M1 |
| E9a-h | S1 | L9 | M1 |
| E10a-h | S2 | L1 | M1 |
| E11a-h | S2 | L2 | M1 |
| E12a-h | S2 | L3 | M1 |
| E13a-h | S2 | L4 | M1 |
| E14a-h | S2 | L5 | M1 |
| E15a-h | S2 | L6 | M1 |
| E16a-h | S2 | L7 | M1 |
| E17a-h | S2 | L8 | M1 |
| E18a-h | S2 | L9 | M1 |
| E19a-h | S3 | L1 | M1 |
| E20a-h | S3 | L2 | M1 |
| E21a-h | S3 | L3 | M1 |
| E22a-h | S3 | L4 | M1 |
| E23a-h | S3 | L5 | M1 |
| E24a-h | S3 | L6 | M1 |
| E25a-h | S3 | L7 | M1 |
| E26a-h | S3 | L8 | M1 |
| E27a-h | S3 | L9 | M1 |
| E28a-h | S4 | L1 | M1 |
| E29a-h | S4 | L2 | M1 |
| E30a-h | S4 | L3 | M1 |
| E31a-h | S4 | L4 | M1 |
| E32a-h | S4 | L5 | M1 |
| E33a-h | S4 | L6 | M1 |
| E34a-h | S4 | L7 | M1 |
| E35a-h | S4 | L8 | M1 |
| E36a-h | S4 | L9 | M1 |
| E37a-h | S5 | L1 | M1 |
| E38a-h | S5 | L2 | M1 |
| E39a-h | S5 | L3 | M1 |
| E40a-h | S5 | L4 | M1 |
| E41a-h | S5 | L5 | M1 |
| E42a-h | S5 | L6 | M1 |
| E43a-h | S5 | L7 | M1 |
| E44a-h | S5 | L8 | M1 |
| E45a-h | S5 | L9 | M1 |
| E46a-h | S6 | L1 | M1 |
| E47a-h | S6 | L2 | M1 |
| E48a-h | S6 | L3 | M1 |
| E49a-h | S6 | L4 | M1 |
| E50a-h | S6 | L5 | M1 |
| E51a-h | S6 | L6 | M1 |
| E52a-h | S6 | L7 | M1 |
| E53a-h | S6 | L8 | M1 |
| E54a-h | S6 | L9 | M1 |
| E55a-h | S7 | L1 | M1 |
| E56a-h | S7 | L2 | M1 |
| E57a-h | S7 | L3 | M1 |
| E58a-h | S7 | L4 | M1 |
| E59a-h | S7 | L5 | M1 |
| E60a-h | S7 | L6 | M1 |
| E61a-h | S7 | L7 | M1 |
| E62a-h | S7 | L8 | M1 |
| E63a-h | S7 | L9 | M1 |
| E64a-h | S8 | L1 | M1 |
| E65a-h | S8 | L2 | M1 |
| E66a-h | S8 | L3 | M1 |
| E67a-h | S8 | L4 | M1 |
| E68a-h | S8 | L5 | M1 |
| E69a-h | S8 | L6 | M1 |
| E70a-h | S8 | L7 | M1 |
| E71a-h | S8 | L8 | M1 |
| E72a-h | S8 | L9 | M1 |
| E73a-h | S9 | L1 | M1 |
| E74a-h | S9 | L2 | M1 |
| E75a-h | S9 | L3 | M1 |
| E76a-h | S9 | L4 | M1 |
| E77a-h | S9 | L5 | M1 |
| E78a-h | S9 | L6 | M1 |
| E79a-h | S9 | L7 | M1 |
| E80a-h | S9 | L8 | M1 |
| E81a-h | S9 | L9 | M1 |
| E82a-h | S10 | L1 | M1 |
| E83a-h | S10 | L2 | M1 |
| E84a-h | S10 | L3 | M1 |
| E85a-h | S10 | L4 | M1 |
| E86a-h | S10 | L5 | M1 |
| E87a-h | S10 | L6 | M1 |
| E88a-h | S10 | L7 | M1 |
| E89a-h | S10 | L8 | M1 |
| E90a-h | S10 | L9 | M1 |
| E91a-h | S11 | L1 | M1 |
| E92a-h | S11 | L2 | M1 |
| E93a-h | S11 | L3 | M1 |
| E94a-h | S11 | L4 | M1 |
| E95a-h | S11 | L5 | M1 |
| E96a-h | S11 | L6 | M1 |
| E97a-h | S11 | L7 | M1 |
| E98a-h | S11 | L8 | M1 |
| E99a-h | S11 | L9 | M1 |
| E100a-h | S12 | L1 | M1 |
| E101a-h | S12 | L2 | M1 |
| E102a-h | S12 | L3 | M1 |
| E103a-h | S12 | L4 | M1 |
| E104a-h | S12 | L5 | M1 |
| E105a-h | S12 | L6 | M1 |
| E106a-h | S12 | L7 | M1 |
| E107a-h | S12 | L8 | M1 |
| E108a-h | S12 | L9 | M1 |
| E109a-h | S13 | L1 | M1 |
| E110a-h | S13 | L2 | M1 |
| E111a-h | S13 | L3 | M1 |
| E112a-h | S13 | L4 | M1 |
| E113a-h | S13 | L5 | M1 |
| E114a-h | S13 | L6 | M1 |
| E115a-h | S13 | L7 | M1 |
| E116a-h | S13 | L8 | M1 |
| E117a-h | S13 | L9 | M1 |
| E118a-h | S14 | L1 | M1 |
| E119a-h | S14 | L2 | M1 |
| E120a-h | S14 | L3 | M1 |
| E121a-h | S14 | L4 | M1 |
| E122a-h | S14 | L5 | M1 |
| E123a-h | S14 | L6 | M1 |
| E124a-h | S14 | L7 | M1 |
| E125a-h | S14 | L8 | M1 |
| E126a-h | S14 | L9 | M1 |
| E127a-h | S15 | L1 | M1 |
| E128a-h | S15 | L2 | M1 |
| E129a-h | S15 | L3 | M1 |
| E130a-h | S15 | L4 | M1 |
| E131a-h | S15 | L5 | M1 |
| E132a-h | S15 | L6 | M1 |
| E133a-h | S15 | L7 | M1 |
| E134a-h | S15 | L8 | M1 |
| E135a-h | S15 | L9 | M1 |
| E136a-h | S16 | L1 | M1 |
| E137a-h | S16 | L2 | M1 |
| E138a-h | S16 | L3 | M1 |
| E139a-h | S16 | L4 | M1 |
| E140a-h | S16 | L5 | M1 |
| E141a-h | S16 | L6 | M1 |
| E142a-h | S16 | L7 | M1 |
| E143a-h | S16 | L8 | M1 |
| E144a-h | S16 | L9 | M1 |
| E145a-h | S1 | L1 | M2 |
| E146a-h | S1 | L2 | M2 |
| E147a-h | S1 | L3 | M2 |
| E148a-h | S1 | L4 | M2 |
| E149a-h | S1 | L5 | M2 |
| E150a-h | S1 | L6 | M2 |
| E151a-h | S1 | L7 | M2 |
| E152a-h | S1 | L8 | M2 |
| E153a-h | S1 | L9 | M2 |
| E154a-h | S2 | L1 | M2 |
| E155a-h | S2 | L2 | M2 |
| E156a-h | S2 | L3 | M2 |
| E157a-h | S2 | L4 | M2 |
| E158a-h | S2 | L5 | M2 |
| E159a-h | S2 | L6 | M2 |
| E160a-h | S2 | L7 | M2 |
| E161a-h | S2 | L8 | M2 |
| E162a-h | S2 | L9 | M2 |
| E163a-h | S3 | L1 | M2 |
| E164a-h | S3 | L2 | M2 |
| E165a-h | S3 | L3 | M2 |
| E166a-h | S3 | L4 | M2 |
| E167a-h | S3 | L5 | M2 |
| E168a-h | S3 | L6 | M2 |
| E169a-h | S3 | L7 | M2 |
| E170a-h | S3 | L8 | M2 |
| E171a-h | S3 | L9 | M2 |
| E172a-h | S4 | L1 | M2 |
| E173a-h | S4 | L2 | M2 |
| E174a-h | S4 | L3 | M2 |
| E175a-h | S4 | L4 | M2 |
| E176a-h | S4 | L5 | M2 |
| E177a-h | S4 | L6 | M2 |
| E178a-h | S4 | L7 | M2 |
| E179a-h | S4 | L8 | M2 |
| E180a-h | S4 | L9 | M2 |
| E181a-h | S5 | L1 | M2 |
| E182a-h | S5 | L2 | M2 |
| E183a-h | S5 | L3 | M2 |
| E184a-h | S5 | L4 | M2 |
| E185a-h | S5 | L5 | M2 |
| E186a-h | S5 | L6 | M2 |
| E187a-h | S5 | L7 | M2 |
| E188a-h | S5 | L8 | M2 |
| E189a-h | S5 | L9 | M2 |
| E190a-h | S6 | L1 | M2 |
| E191a-h | S6 | L2 | M2 |
| E192a-h | S6 | L3 | M2 |
| E193a-h | S6 | L4 | M2 |
| E194a-h | S6 | L5 | M2 |
| E195a-h | S6 | L6 | M2 |
| E196a-h | S6 | L7 | M2 |
| E197a-h | S6 | L8 | M2 |
| E198a-h | S6 | L9 | M2 |
| E199a-h | S7 | L1 | M2 |
| E200a-h | S7 | L2 | M2 |
| E201a-h | S7 | L3 | M2 |
| E202a-h | S7 | L4 | M2 |
| E203a-h | S7 | L5 | M2 |
| E204a-h | S7 | L6 | M2 |
| E205a-h | S7 | L7 | M2 |
| E206a-h | S7 | L8 | M2 |
| E207a-h | S7 | L9 | M2 |
| E208a-h | S8 | L1 | M2 |
| E209a-h | S8 | L2 | M2 |
| E210a-h | S8 | L3 | M2 |
| E211a-h | S8 | L4 | M2 |
| E212a-h | S8 | L5 | M2 |
| E213a-h | S8 | L6 | M2 |
| E214a-h | S8 | L7 | M2 |
| E215a-h | S8 | L8 | M2 |
| E216a-h | S8 | L9 | M2 |
| E217a-h | S9 | L1 | M2 |
| E218a-h | S9 | L2 | M2 |
| E219a-h | S9 | L3 | M2 |
| E220a-h | S9 | L4 | M2 |
| E221a-h | S9 | L5 | M2 |
| E222a-h | S9 | L6 | M2 |
| E223a-h | S9 | L7 | M2 |
| E224a-h | S9 | L8 | M2 |
| E225a-h | S9 | L9 | M2 |
| E226a-h | S10 | L1 | M2 |
| E227a-h | S10 | L2 | M2 |
| E228a-h | S10 | L3 | M2 |
| E229a-h | S10 | L4 | M2 |
| E230a-h | S10 | L5 | M2 |
| E231a-h | S10 | L6 | M2 |
| E232a-h | S10 | L7 | M2 |
| E233a-h | S10 | L8 | M2 |
| E234a-h | S10 | L9 | M2 |
| E235a-h | S11 | L1 | M2 |
| E236a-h | S11 | L2 | M2 |
| E237a-h | S11 | L3 | M2 |
| E238a-h | S11 | L4 | M2 |
| E239a-h | S11 | L5 | M2 |
| E240a-h | S11 | L6 | M2 |
| E241a-h | S11 | L7 | M2 |
| E242a-h | S11 | L8 | M2 |
| E243a-h | S11 | L9 | M2 |
| E244a-h | S12 | L1 | M2 |
| E245a-h | S12 | L2 | M2 |
| E246a-h | S12 | L3 | M2 |
| E247a-h | S12 | L4 | M2 |
| E248a-h | S12 | L5 | M2 |
| E249a-h | S12 | L6 | M2 |
| E250a-h | S12 | L7 | M2 |
| E251a-h | S12 | L8 | M2 |
| E252a-h | S12 | L9 | M2 |
| E253a-h | S13 | L1 | M2 |
| E254a-h | S13 | L2 | M2 |
| E255a-h | S13 | L3 | M2 |
| E256a-h | S13 | L4 | M2 |
| E257a-h | S13 | L5 | M2 |
| E258a-h | S13 | L6 | M2 |
| E259a-h | S13 | L7 | M2 |
| E260a-h | S13 | L8 | M2 |
| E261a-h | S13 | L9 | M2 |
| E262a-h | S14 | L1 | M2 |
| E263a-h | S14 | L2 | M2 |
| E264a-h | S14 | L3 | M2 |
| E265a-h | S14 | L4 | M2 |
| E266a-h | S14 | L5 | M2 |
| E267a-h | S14 | L6 | M2 |
| E268a-h | S14 | L7 | M2 |
| E269a-h | S14 | L8 | M2 |
| E270a-h | S14 | L9 | M2 |
| E271a-h | S15 | L1 | M2 |
| E272a-h | S15 | L2 | M2 |
| E273a-h | S15 | L3 | M2 |
| E274a-h | S15 | L4 | M2 |
| E275a-h | S15 | L5 | M2 |
| E276a-h | S15 | L6 | M2 |
| E277a-h | S15 | L7 | M2 |
| E278a-h | S15 | L8 | M2 |
| E279a-h | S15 | L9 | M2 |
| E280a-h | S16 | L1 | M2 |
| E281a-h | S16 | L2 | M2 |
| E282a-h | S16 | L3 | M2 |
| E283a-h | S16 | L4 | M2 |
| E284a-h | S16 | L5 | M2 |
| E285a-h | S16 | L6 | M2 |
| E286a-h | S16 | L7 | M2 |
| E287a-h | S16 | L8 | M2 |
| E288a-h | S16 | L9 | M2 |
| E289a-h | S1 | L1 | M3 |
| E290a-h | S1 | L2 | M3 |
| E291a-h | S1 | L3 | M3 |
| E292a-h | S1 | L4 | M3 |
| E293a-h | S1 | L5 | M3 |
| E294a-h | S1 | L6 | M3 |
| E295a-h | S1 | L7 | M3 |
| E296a-h | S1 | L8 | M3 |
| E297a-h | S1 | L9 | M3 |
| E298a-h | S2 | L1 | M3 |
| E299a-h | S2 | L2 | M3 |
| E300a-h | S2 | L3 | M3 |
| E301a-h | S2 | L4 | M3 |
| E302a-h | S2 | L5 | M3 |
| E303a-h | S2 | L6 | M3 |
| E304a-h | S2 | L7 | M3 |
| E305a-h | S2 | L8 | M3 |
| E306a-h | S2 | L9 | M3 |
| E307a-h | S3 | L1 | M3 |
| E308a-h | S3 | L2 | M3 |
| E309a-h | S3 | L3 | M3 |
| E310a-h | S3 | L4 | M3 |
| E311a-h | S3 | L5 | M3 |
| E312a-h | S3 | L6 | M3 |
| E313a-h | S3 | L7 | M3 |
| E314a-h | S3 | L8 | M3 |
| E315a-h | S3 | L9 | M3 |
| E316a-h | S4 | L1 | M3 |
| E317a-h | S4 | L2 | M3 |
| E318a-h | S4 | L3 | M3 |
| E319a-h | S4 | L4 | M3 |
| E320a-h | S4 | L5 | M3 |
| E321a-h | S4 | L6 | M3 |
| E322a-h | S4 | L7 | M3 |
| E323a-h | S4 | L8 | M3 |
| E324a-h | S4 | L9 | M3 |
| E325a-h | S5 | L1 | M3 |
| E326a-h | S5 | L2 | M3 |
| E327a-h | S5 | L3 | M3 |
| E328a-h | S5 | L4 | M3 |
| E329a-h | S5 | L5 | M3 |
| E330a-h | S5 | L6 | M3 |
| E331a-h | S5 | L7 | M3 |
| E332a-h | S5 | L8 | M3 |
| E333a-h | S5 | L9 | M3 |
| E334a-h | S6 | L1 | M3 |
| E335a-h | S6 | L2 | M3 |
| E336a-h | S6 | L3 | M3 |
| E337a-h | S6 | L4 | M3 |
| E338a-h | S6 | L5 | M3 |
| E339a-h | S6 | L6 | M3 |
| E340a-h | S6 | L7 | M3 |
| E341a-h | S6 | L8 | M3 |
| E342a-h | S6 | L9 | M3 |
| E343a-h | S7 | L1 | M3 |
| E344a-h | S7 | L2 | M3 |
| E345a-h | S7 | L3 | M3 |
| E346a-h | S7 | L4 | M3 |
| E347a-h | S7 | L5 | M3 |
| E348a-h | S7 | L6 | M3 |
| E349a-h | S7 | L7 | M3 |
| E350a-h | S7 | L8 | M3 |
| E351a-h | S7 | L9 | M3 |
| E352a-h | S8 | L1 | M3 |
| E353a-h | S8 | L2 | M3 |
| E354a-h | S8 | L3 | M3 |
| E355a-h | S8 | L4 | M3 |
| E356a-h | S8 | L5 | M3 |
| E357a-h | S8 | L6 | M3 |
| E358a-h | S8 | L7 | M3 |
| E359a-h | S8 | L8 | M3 |
| E360a-h | S8 | L9 | M3 |
| E361a-h | S9 | L1 | M3 |
| E362a-h | S9 | L2 | M3 |
| E363a-h | S9 | L3 | M3 |
| E364a-h | S9 | L4 | M3 |
| E365a-h | S9 | L5 | M3 |
| E366a-h | S9 | L6 | M3 |
| E367a-h | S9 | L7 | M3 |
| E368a-h | S9 | L8 | M3 |
| E369a-h | S9 | L9 | M3 |
| E370a-h | S10 | L1 | M3 |
| E371a-h | S10 | L2 | M3 |
| E372a-h | S10 | L3 | M3 |
| E373a-h | S10 | L4 | M3 |
| E374a-h | S10 | L5 | M3 |
| E375a-h | S10 | L6 | M3 |
| E376a-h | S10 | L7 | M3 |
| E377a-h | S10 | L8 | M3 |
| E378a-h | S10 | L9 | M3 |
| E379a-h | S11 | L1 | M3 |
| E380a-h | S11 | L2 | M3 |
| E381a-h | S11 | L3 | M3 |
| E382a-h | S11 | L4 | M3 |
| E383a-h | S11 | L5 | M3 |
| E384a-h | S11 | L6 | M3 |
| E385a-h | S11 | L7 | M3 |
| E386a-h | S11 | L8 | M3 |
| E387a-h | S11 | L9 | M3 |
| E388a-h | S12 | L1 | M3 |
| E389a-h | S12 | L2 | M3 |
| E390a-h | S12 | L3 | M3 |
| E391a-h | S12 | L4 | M3 |
| E392a-h | S12 | L5 | M3 |
| E393a-h | S12 | L6 | M3 |
| E394a-h | S12 | L7 | M3 |
| E395a-h | S12 | L8 | M3 |
| E396a-h | S12 | L9 | M3 |
| E397a-h | S13 | L1 | M3 |
| E398a-h | S13 | L2 | M3 |
| E399a-h | S13 | L3 | M3 |
| E400a-h | S13 | L4 | M3 |
| E401a-h | S13 | L5 | M3 |
| E402a-h | S13 | L6 | M3 |
| E403a-h | S13 | L7 | M3 |
| E404a-h | S13 | L8 | M3 |
| E405a-h | S13 | L9 | M3 |
| E406a-h | S14 | L1 | M3 |
| E407a-h | S14 | L2 | M3 |
| E408a-h | S14 | L3 | M3 |
| E409a-h | S14 | L4 | M3 |
| E410a-h | S14 | L5 | M3 |
| E411a-h | S14 | L6 | M3 |
| E412a-h | S14 | L7 | M3 |
| E413a-h | S14 | L8 | M3 |
| E414a-h | S14 | L9 | M3 |
| E415a-h | S15 | L1 | M3 |
| E416a-h | S15 | L2 | M3 |
| E417a-h | S15 | L3 | M3 |
| E418a-h | S15 | L4 | M3 |
| E419a-h | S15 | L5 | M3 |
| E420a-h | S15 | L6 | M3 |
| E421a-h | S15 | L7 | M3 |
| E422a-h | S15 | L8 | M3 |
| E423a-h | S15 | L9 | M3 |
| E424a-h | S16 | L1 | M3 |
| E425a-h | S16 | L2 | M3 |
| E426a-h | S16 | L3 | M3 |
| E427a-h | S16 | L4 | M3 |
| E428a-h | S16 | L5 | M3 |
| E429a-h | S16 | L6 | M3 |
| E430a-h | S16 | L7 | M3 |
| E431a-h | S16 | L8 | M3 |
| E432a-h | S16 | L9 | M3 |
| E433a-h | S1 | L1 | M4 |
| E434a-h | S1 | L2 | M4 |
| E435a-h | S1 | L3 | M4 |
| E436a-h | S1 | L4 | M4 |
| E437a-h | S1 | L5 | M4 |
| E438a-h | S1 | L6 | M4 |
| E439a-h | S1 | L7 | M4 |
| E440a-h | S1 | L8 | M4 |
| E441a-h | S1 | L9 | M4 |
| E442a-h | S2 | L1 | M4 |
| E443a-h | S2 | L2 | M4 |
| E444a-h | S2 | L3 | M4 |
| E445a-h | S2 | L4 | M4 |
| E446a-h | S2 | L5 | M4 |
| E447a-h | S2 | L6 | M4 |
| E448a-h | S2 | L7 | M4 |
| E449a-h | S2 | L8 | M4 |
| E450a-h | S2 | L9 | M4 |
| E451a-h | S3 | L1 | M4 |
| E452a-h | S3 | L2 | M4 |
| E453a-h | S3 | L3 | M4 |
| E454a-h | S3 | L4 | M4 |
| E455a-h | S3 | L5 | M4 |
| E456a-h | S3 | L6 | M4 |
| E457a-h | S3 | L7 | M4 |
| E458a-h | S3 | L8 | M4 |
| E459a-h | S3 | L9 | M4 |
| E460a-h | S4 | L1 | M4 |
| E461a-h | S4 | L2 | M4 |
| E462a-h | S4 | L3 | M4 |
| E463a-h | S4 | L4 | M4 |
| E464a-h | S4 | L5 | M4 |
| E465a-h | S4 | L6 | M4 |
| E466a-h | S4 | L7 | M4 |
| E467a-h | S4 | L8 | M4 |
| E468a-h | S4 | L9 | M4 |
| E469a-h | S5 | L1 | M4 |
| E470a-h | S5 | L2 | M4 |
| E471a-h | S5 | L3 | M4 |
| E472a-h | S5 | L4 | M4 |
| E473a-h | S5 | L5 | M4 |
| E474a-h | S5 | L6 | M4 |
| E475a-h | S5 | L7 | M4 |
| E476a-h | S5 | L8 | M4 |
| E477a-h | S5 | L9 | M4 |
| E478a-h | S6 | L1 | M4 |
| E479a-h | S6 | L2 | M4 |
| E480a-h | S6 | L3 | M4 |
| E481a-h | S6 | L4 | M4 |
| E482a-h | S6 | L5 | M4 |
| E483a-h | S6 | L6 | M4 |
| E484a-h | S6 | L7 | M4 |
| E485a-h | S6 | L8 | M4 |
| E486a-h | S6 | L9 | M4 |
| E487a-h | S7 | L1 | M4 |
| E488a-h | S7 | L2 | M4 |
| E489a-h | S7 | L3 | M4 |
| E490a-h | S7 | L4 | M4 |
| E491a-h | S7 | L5 | M4 |
| E492a-h | S7 | L6 | M4 |
| E493a-h | S7 | L7 | M4 |
| E494a-h | S7 | L8 | M4 |
| E495a-h | S7 | L9 | M4 |
| E496a-h | S8 | L1 | M4 |
| E497a-h | S8 | L2 | M4 |
| E498a-h | S8 | L3 | M4 |
| E499a-h | S8 | L4 | M4 |
| E500a-h | S8 | L5 | M4 |
| E501a-h | S8 | L6 | M4 |
| E502a-h | S8 | L7 | M4 |
| E503a-h | S8 | L8 | M4 |
| E504a-h | S8 | L9 | M4 |
| E505a-h | S9 | L1 | M4 |
| E506a-h | S9 | L2 | M4 |
| E507a-h | S9 | L3 | M4 |
| E508a-h | S9 | L4 | M4 |
| E509a-h | S9 | L5 | M4 |
| E510a-h | S9 | L6 | M4 |
| E511a-h | S9 | L7 | M4 |
| E512a-h | S9 | L8 | M4 |
| E513a-h | S9 | L9 | M4 |
| E514a-h | S10 | L1 | M4 |
| E515a-h | S10 | L2 | M4 |
| E516a-h | S10 | L3 | M4 |
| E517a-h | S10 | L4 | M4 |
| E518a-h | S10 | L5 | M4 |
| E519a-h | S10 | L6 | M4 |
| E520a-h | S10 | L7 | M4 |
| E521a-h | S10 | L8 | M4 |
| E522a-h | S10 | L9 | M4 |
| E523a-h | S11 | L1 | M4 |
| E524a-h | S11 | L2 | M4 |
| E525a-h | S11 | L3 | M4 |
| E526a-h | S11 | L4 | M4 |
| E527a-h | S11 | L5 | M4 |
| E528a-h | S11 | L6 | M4 |
| E529a-h | S11 | L7 | M4 |
| E530a-h | S11 | L8 | M4 |
| E531a-h | S11 | L9 | M4 |
| E532a-h | S12 | L1 | M4 |
| E533a-h | S12 | L2 | M4 |
| E534a-h | S12 | L3 | M4 |
| E535a-h | S12 | L4 | M4 |
| E536a-h | S12 | L5 | M4 |
| E537a-h | S12 | L6 | M4 |
| E538a-h | S12 | L7 | M4 |
| E539a-h | S12 | L8 | M4 |
| E540a-h | S12 | L9 | M4 |
| E541a-h | S13 | L1 | M4 |
| E542a-h | S13 | L2 | M4 |
| E543a-h | S13 | L3 | M4 |
| E544a-h | S13 | L4 | M4 |
| E545a-h | S13 | L5 | M4 |
| E546a-h | S13 | L6 | M4 |
| E547a-h | S13 | L7 | M4 |
| E548a-h | S13 | L8 | M4 |
| E549a-h | S13 | L9 | M4 |
| E550a-h | S14 | L1 | M4 |
| E551a-h | S14 | L2 | M4 |
| E552a-h | S14 | L3 | M4 |
| E553a-h | S14 | L4 | M4 |
| E554a-h | S14 | L5 | M4 |
| E555a-h | S14 | L6 | M4 |
| E556a-h | S14 | L7 | M4 |
| E557a-h | S14 | L8 | M4 |
| E558a-h | S14 | L9 | M4 |
| E559a-h | S15 | L1 | M4 |
| E560a-h | S15 | L2 | M4 |
| E561a-h | S15 | L3 | M4 |
| E562a-h | S15 | L4 | M4 |
| E563a-h | S15 | L5 | M4 |
| E564a-h | S15 | L6 | M4 |
| E565a-h | S15 | L7 | M4 |
| E566a-h | S15 | L8 | M4 |
| E567a-h | S15 | L9 | M4 |
| E568a-h | S16 | L1 | M4 |
| E569a-h | S16 | L2 | M4 |
| E570a-h | S16 | L3 | M4 |
| E571a-h | S16 | L4 | M4 |
| E572a-h | S16 | L5 | M4 |
| E573a-h | S16 | L6 | M4 |
| E574a-h | S16 | L7 | M4 |
| E575a-h | S16 | L8 | M4 |
| E576a-h | S16 | L9 | M4 |
| E577a-h | S1 | L1 | M5 |
| E578a-h | S1 | L2 | M5 |
| E579a-h | S1 | L3 | M5 |
| E580a-h | S1 | L4 | M5 |
| E581a-h | S1 | L5 | M5 |
| E582a-h | S1 | L6 | M5 |
| E583a-h | S1 | L7 | M5 |
| E584a-h | S1 | L8 | M5 |
| E585a-h | S1 | L9 | M5 |
| E586a-h | S2 | L1 | M5 |
| E587a-h | S2 | L2 | M5 |
| E588a-h | S2 | L3 | M5 |
| E589a-h | S2 | L4 | M5 |
| E590a-h | S2 | L5 | M5 |
| E591a-h | S2 | L6 | M5 |
| E592a-h | S2 | L7 | M5 |
| E593a-h | S2 | L8 | M5 |
| E594a-h | S2 | L9 | M5 |
| E595a-h | S3 | L1 | M5 |
| E596a-h | S3 | L2 | M5 |
| E597a-h | S3 | L3 | M5 |
| E598a-h | S3 | L4 | M5 |
| E599a-h | S3 | L5 | M5 |
| E600a-h | S3 | L6 | M5 |
| E601a-h | S3 | L7 | M5 |
| E602a-h | S3 | L8 | M5 |
| E603a-h | S3 | L9 | M5 |
| E604a-h | S4 | L1 | M5 |
| E605a-h | S4 | L2 | M5 |
| E606a-h | S4 | L3 | M5 |
| E607a-h | S4 | L4 | M5 |
| E608a-h | S4 | L5 | M5 |
| E609a-h | S4 | L6 | M5 |
| E610a-h | S4 | L7 | M5 |
| E611a-h | S4 | L8 | M5 |
| E612a-h | S4 | L9 | M5 |
| E613a-h | S5 | L1 | M5 |
| E614a-h | S5 | L2 | M5 |
| E615a-h | S5 | L3 | M5 |
| E616a-h | S5 | L4 | M5 |
| E617a-h | S5 | L5 | M5 |
| E618a-h | S5 | L6 | M5 |
| E619a-h | S5 | L7 | M5 |
| E620a-h | S5 | L8 | M5 |
| E621a-h | S5 | L9 | M5 |
| E622a-h | S6 | L1 | M5 |
| E623a-h | S6 | L2 | M5 |
| E624a-h | S6 | L3 | M5 |
| E625a-h | S6 | L4 | M5 |
| E626a-h | S6 | L5 | M5 |
| E627a-h | S6 | L6 | M5 |
| E628a-h | S6 | L7 | M5 |
| E629a-h | S6 | L8 | M5 |
| E630a-h | S6 | L9 | M5 |
| E631a-h | S7 | L1 | M5 |
| E632a-h | S7 | L2 | M5 |
| E633a-h | S7 | L3 | M5 |
| E634a-h | S7 | L4 | M5 |
| E635a-h | S7 | L5 | M5 |
| E636a-h | S7 | L6 | M5 |
| E637a-h | S7 | L7 | M5 |
| E638a-h | S7 | L8 | M5 |
| E639a-h | S7 | L9 | M5 |
| E640a-h | S8 | L1 | M5 |
| E641a-h | S8 | L2 | M5 |
| E642a-h | S8 | L3 | M5 |
| E643a-h | S8 | L4 | M5 |
| E644a-h | S8 | L5 | M5 |
| E645a-h | S8 | L6 | M5 |
| E646a-h | S8 | L7 | M5 |
| E647a-h | S8 | L8 | M5 |
| E648a-h | S8 | L9 | M5 |
| E649a-h | S9 | L1 | M5 |
| E650a-h | S9 | L2 | M5 |
| E651a-h | S9 | L3 | M5 |
| E652a-h | S9 | L4 | M5 |
| E653a-h | S9 | L5 | M5 |
| E654a-h | S9 | L6 | M5 |
| E655a-h | S9 | L7 | M5 |
| E656a-h | S9 | L8 | M5 |
| E657a-h | S9 | L9 | M5 |
| E658a-h | S10 | L1 | M5 |
| E659a-h | S10 | L2 | M5 |
| E660a-h | S10 | L3 | M5 |
| E661a-h | S10 | L4 | M5 |
| E662a-h | S10 | L5 | M5 |
| E663a-h | S10 | L6 | M5 |
| E664a-h | S10 | L7 | M5 |
| E665a-h | S10 | L8 | M5 |
| E666a-h | S10 | L9 | M5 |
| E667a-h | S11 | L1 | M5 |
| E668a-h | S11 | L2 | M5 |
| E669a-h | S11 | L3 | M5 |
| E670a-h | S11 | L4 | M5 |
| E671a-h | S11 | L5 | M5 |
| E672a-h | S11 | L6 | M5 |
| E673a-h | S11 | L7 | M5 |
| E674a-h | S11 | L8 | M5 |
| E675a-h | S11 | L9 | M5 |
| E676a-h | S12 | L1 | M5 |
| E677a-h | S12 | L2 | M5 |
| E678a-h | S12 | L3 | M5 |
| E679a-h | S12 | L4 | M5 |
| E680a-h | S12 | L5 | M5 |
| E681a-h | S12 | L6 | M5 |
| E682a-h | S12 | L7 | M5 |
| E683a-h | S12 | L8 | M5 |
| E684a-h | S12 | L9 | M5 |
| E685a-h | S13 | L1 | M5 |
| E686a-h | S13 | L2 | M5 |
| E687a-h | S13 | L3 | M5 |
| E688a-h | S13 | L4 | M5 |
| E689a-h | S13 | L5 | M5 |
| E690a-h | S13 | L6 | M5 |
| E691a-h | S13 | L7 | M5 |
| E692a-h | S13 | L8 | M5 |
| E693a-h | S13 | L9 | M5 |
| E694a-h | S14 | L1 | M5 |
| E695a-h | S14 | L2 | M5 |
| E696a-h | S14 | L3 | M5 |
| E697a-h | S14 | L4 | M5 |
| E698a-h | S14 | L5 | M5 |
| E699a-h | S14 | L6 | M5 |
| E700a-h | S14 | L7 | M5 |
| E701a-h | S14 | L8 | M5 |
| E702a-h | S14 | L9 | M5 |
| E703a-h | S15 | L1 | M5 |
| E704a-h | S15 | L2 | M5 |
| E705a-h | S15 | L3 | M5 |
| E706a-h | S15 | L4 | M5 |
| E707a-h | S15 | L5 | M5 |
| E708a-h | S15 | L6 | M5 |
| E709a-h | S15 | L7 | M5 |
| E710a-h | S15 | L8 | M5 |
| E711a-h | S15 | L9 | M5 |
| E712a-h | S16 | L1 | M5 |
| E713a-h | S16 | L2 | M5 |
| E714a-h | S16 | L3 | M5 |
| E715a-h | S16 | L4 | M5 |
| E716a-h | S16 | L5 | M5 |
| E717a-h | S16 | L6 | M5 |
| E718a-h | S16 | L7 | M5 |
| E719a-h | S16 | L8 | M5 |
| E720a-h | S16 | L9 | M5 |
| E721a-h | S1 | L1 | M6 |
| E722a-h | S1 | L2 | M6 |
| E723a-h | S1 | L3 | M6 |
| E724a-h | S1 | L4 | M6 |
| E725a-h | S1 | L5 | M6 |
| E726a-h | S1 | L6 | M6 |
| E727a-h | S1 | L7 | M6 |
| E728a-h | S1 | L8 | M6 |
| E729a-h | S1 | L9 | M6 |
| E730a-h | S2 | L1 | M6 |
| E731a-h | S2 | L2 | M6 |
| E732a-h | S2 | L3 | M6 |
| E733a-h | S2 | L4 | M6 |
| E734a-h | S2 | L5 | M6 |
| E735a-h | S2 | L6 | M6 |
| E736a-h | S2 | L7 | M6 |
| E737a-h | S2 | L8 | M6 |
| E738a-h | S2 | L9 | M6 |
| E739a-h | S3 | L1 | M6 |
| E740a-h | S3 | L2 | M6 |
| E741a-h | S3 | L3 | M6 |
| E742a-h | S3 | L4 | M6 |
| E743a-h | S3 | L5 | M6 |
| E744a-h | S3 | L6 | M6 |
| E745a-h | S3 | L7 | M6 |
| E746a-h | S3 | L8 | M6 |
| E747a-h | S3 | L9 | M6 |
| E748a-h | S4 | L1 | M6 |
| E749a-h | S4 | L2 | M6 |
| E750a-h | S4 | L3 | M6 |
| E751a-h | S4 | L4 | M6 |
| E752a-h | S4 | L5 | M6 |
| E753a-h | S4 | L6 | M6 |
| E754a-h | S4 | L7 | M6 |
| E755a-h | S4 | L8 | M6 |
| E756a-h | S4 | L9 | M6 |
| E757a-h | S5 | L1 | M6 |
| E758a-h | S5 | L2 | M6 |
| E759a-h | S5 | L3 | M6 |
| E760a-h | S5 | L4 | M6 |
| E761a-h | S5 | L5 | M6 |
| E762a-h | S5 | L6 | M6 |
| E763a-h | S5 | L7 | M6 |
| E764a-h | S5 | L8 | M6 |
| E765a-h | S5 | L9 | M6 |
| E766a-h | S6 | L1 | M6 |
| E767a-h | S6 | L2 | M6 |
| E768a-h | S6 | L3 | M6 |
| E769a-h | S6 | L4 | M6 |
| E770a-h | S6 | L5 | M6 |
| E771a-h | S6 | L6 | M6 |
| E772a-h | S6 | L7 | M6 |
| E773a-h | S6 | L8 | M6 |
| E774a-h | S6 | L9 | M6 |
| E775a-h | S7 | L1 | M6 |
| E776a-h | S7 | L2 | M6 |
| E777a-h | S7 | L3 | M6 |
| E778a-h | S7 | L4 | M6 |
| E779a-h | S7 | L5 | M6 |
| E780a-h | S7 | L6 | M6 |
| E781a-h | S7 | L7 | M6 |
| E782a-h | S7 | L8 | M6 |
| E783a-h | S7 | L9 | M6 |
| E784a-h | S8 | L1 | M6 |
| E785a-h | S8 | L2 | M6 |
| E786a-h | S8 | L3 | M6 |
| E787a-h | S8 | L4 | M6 |
| E788a-h | S8 | L5 | M6 |
| E789a-h | S8 | L6 | M6 |
| E790a-h | S8 | L7 | M6 |
| E791a-h | S8 | L8 | M6 |
| E792a-h | S8 | L9 | M6 |
| E793a-h | S9 | L1 | M6 |
| E794a-h | S9 | L2 | M6 |
| E795a-h | S9 | L3 | M6 |
| E796a-h | S9 | L4 | M6 |
| E797a-h | S9 | L5 | M6 |
| E798a-h | S9 | L6 | M6 |
| E799a-h | S9 | L7 | M6 |
| E800a-h | S9 | L8 | M6 |
| E801a-h | S9 | L9 | M6 |
| E802a-h | S10 | L1 | M6 |
| E803a-h | S10 | L2 | M6 |
| E804a-h | S10 | L3 | M6 |
| E805a-h | S10 | L4 | M6 |
| E806a-h | S10 | L5 | M6 |
| E807a-h | S10 | L6 | M6 |
| E808a-h | S10 | L7 | M6 |
| E809a-h | S10 | L8 | M6 |
| E810a-h | S10 | L9 | M6 |
| E811a-h | S11 | L1 | M6 |
| E812a-h | S11 | L2 | M6 |
| E813a-h | S11 | L3 | M6 |
| E814a-h | S11 | L4 | M6 |
| E815a-h | S11 | L5 | M6 |
| E816a-h | S11 | L6 | M6 |
| E817a-h | S11 | L7 | M6 |
| E818a-h | S11 | L8 | M6 |
| E819a-h | S11 | L9 | M6 |
| E820a-h | S12 | L1 | M6 |
| E821a-h | S12 | L2 | M6 |
| E822a-h | S12 | L3 | M6 |
| E823a-h | S12 | L4 | M6 |
| E824a-h | S12 | L5 | M6 |
| E825a-h | S12 | L6 | M6 |
| E826a-h | S12 | L7 | M6 |
| E827a-h | S12 | L8 | M6 |
| E828a-h | S12 | L9 | M6 |
| E829a-h | S13 | L1 | M6 |
| E830a-h | S13 | L2 | M6 |
| E831a-h | S13 | L3 | M6 |
| E832a-h | S13 | L4 | M6 |
| E833a-h | S13 | L5 | M6 |
| E834a-h | S13 | L6 | M6 |
| E835a-h | S13 | L7 | M6 |
| E836a-h | S13 | L8 | M6 |
| E837a-h | S13 | L9 | M6 |
| E838a-h | S14 | L1 | M6 |
| E839a-h | S14 | L2 | M6 |
| E840a-h | S14 | L3 | M6 |
| E841a-h | S14 | L4 | M6 |
| E842a-h | S14 | L5 | M6 |
| E843a-h | S14 | L6 | M6 |
| E844a-h | S14 | L7 | M6 |
| E845a-h | S14 | L8 | M6 |
| E846a-h | S14 | L9 | M6 |
| E847a-h | S15 | L1 | M6 |
| E848a-h | S15 | L2 | M6 |
| E849a-h | S15 | L3 | M6 |
| E850a-h | S15 | L4 | M6 |
| E851a-h | S15 | L5 | M6 |
| E852a-h | S15 | L6 | M6 |
| E853a-h | S15 | L7 | M6 |
| E854a-h | S15 | L8 | M6 |
| E855a-h | S15 | L9 | M6 |
| E856a-h | S16 | L1 | M6 |
| E857a-h | S16 | L2 | M6 |
| E858a-h | S16 | L3 | M6 |
| E859a-h | S16 | L4 | M6 |
| E860a-h | S16 | L5 | M6 |
| E861a-h | S16 | L6 | M6 |
| E862a-h | S16 | L7 | M6 |
| E863a-h | S16 | L8 | M6 |
| E864a-h | S16 | L9 | M6 |
| E865a-h | S1 | L1 | M7 |
| E866a-h | S1 | L2 | M7 |
| E867a-h | S1 | L3 | M7 |
| E868a-h | S1 | L4 | M7 |
| E869a-h | S1 | L5 | M7 |
| E870a-h | S1 | L6 | M7 |
| E871a-h | S1 | L7 | M7 |
| E872a-h | S1 | L8 | M7 |
| E873a-h | S1 | L9 | M7 |
| E874a-h | S2 | L1 | M7 |
| E875a-h | S2 | L2 | M7 |
| E876a-h | S2 | L3 | M7 |
| E877a-h | S2 | L4 | M7 |
| E878a-h | S2 | L5 | M7 |
| E879a-h | S2 | L6 | M7 |
| E880a-h | S2 | L7 | M7 |
| E881a-h | S2 | L8 | M7 |
| E882a-h | S2 | L9 | M7 |
| E883a-h | S3 | L1 | M7 |
| E884a-h | S3 | L2 | M7 |
| E885a-h | S3 | L3 | M7 |
| E886a-h | S3 | L4 | M7 |
| E887a-h | S3 | L5 | M7 |
| E888a-h | S3 | L6 | M7 |
| E889a-h | S3 | L7 | M7 |
| E890a-h | S3 | L8 | M7 |
| E891a-h | S3 | L9 | M7 |
| E892a-h | S4 | L1 | M7 |
| E893a-h | S4 | L2 | M7 |
| E894a-h | S4 | L3 | M7 |
| E895a-h | S4 | L4 | M7 |
| E896a-h | S4 | L5 | M7 |
| E897a-h | S4 | L6 | M7 |
| E898a-h | S4 | L7 | M7 |
| E899a-h | S4 | L8 | M7 |
| E900a-h | S4 | L9 | M7 |
| E901a-h | S5 | L1 | M7 |
| E902a-h | S5 | L2 | M7 |
| E903a-h | S5 | L3 | M7 |
| E904a-h | S5 | L4 | M7 |
| E905a-h | S5 | L5 | M7 |
| E906a-h | S5 | L6 | M7 |
| E907a-h | S5 | L7 | M7 |
| E908a-h | S5 | L8 | M7 |
| E909a-h | S5 | L9 | M7 |
| E910a-h | S6 | L1 | M7 |
| E911a-h | S6 | L2 | M7 |
| E912a-h | S6 | L3 | M7 |
| E913a-h | S6 | L4 | M7 |
| E914a-h | S6 | L5 | M7 |
| E915a-h | S6 | L6 | M7 |
| E916a-h | S6 | L7 | M7 |
| E917a-h | S6 | L8 | M7 |
| E918a-h | S6 | L9 | M7 |
| E919a-h | S7 | L1 | M7 |
| E920a-h | S7 | L2 | M7 |
| E921a-h | S7 | L3 | M7 |
| E922a-h | S7 | L4 | M7 |
| E923a-h | S7 | L5 | M7 |
| E924a-h | S7 | L6 | M7 |
| E925a-h | S7 | L7 | M7 |
| E926a-h | S7 | L8 | M7 |
| E927a-h | S7 | L9 | M7 |
| E928a-h | S8 | L1 | M7 |
| E929a-h | S8 | L2 | M7 |
| E930a-h | S8 | L3 | M7 |
| E931a-h | S8 | L4 | M7 |
| E932a-h | S8 | L5 | M7 |
| E933a-h | S8 | L6 | M7 |
| E934a-h | S8 | L7 | M7 |
| E935a-h | S8 | L8 | M7 |
| E936a-h | S8 | L9 | M7 |
| E937a-h | S9 | L1 | M7 |
| E938a-h | S9 | L2 | M7 |
| E939a-h | S9 | L3 | M7 |
| E940a-h | S9 | L4 | M7 |
| E941a-h | S9 | L5 | M7 |
| E942a-h | S9 | L6 | M7 |
| E943a-h | S9 | L7 | M7 |
| E944a-h | S9 | L8 | M7 |
| E945a-h | S9 | L9 | M7 |
| E946a-h | S10 | L1 | M7 |
| E947a-h | S10 | L2 | M7 |
| E948a-h | S10 | L3 | M7 |
| E949a-h | S10 | L4 | M7 |
| E950a-h | S10 | L5 | M7 |
| E951a-h | S10 | L6 | M7 |
| E952a-h | S10 | L7 | M7 |
| E953a-h | S10 | L8 | M7 |
| E954a-h | S10 | L9 | M7 |
| E955a-h | S11 | L1 | M7 |
| E956a-h | S11 | L2 | M7 |
| E957a-h | S11 | L3 | M7 |
| E958a-h | S11 | L4 | M7 |
| E959a-h | S11 | L5 | M7 |
| E960a-h | S11 | L6 | M7 |
| E961a-h | S11 | L7 | M7 |
| E962a-h | S11 | L8 | M7 |
| E963a-h | S11 | L9 | M7 |
| E964a-h | S12 | L1 | M7 |
| E965a-h | S12 | L2 | M7 |
| E966a-h | S12 | L3 | M7 |
| E967a-h | S12 | L4 | M7 |
| E968a-h | S12 | L5 | M7 |
| E969a-h | S12 | L6 | M7 |
| E970a-h | S12 | L7 | M7 |
| E971a-h | S12 | L8 | M7 |
| E972a-h | S12 | L9 | M7 |
| E973a-h | S13 | L1 | M7 |
| E974a-h | S13 | L2 | M7 |
| E975a-h | S13 | L3 | M7 |
| E976a-h | S13 | L4 | M7 |
| E977a-h | S13 | L5 | M7 |
| E978a-h | S13 | L6 | M7 |
| E979a-h | S13 | L7 | M7 |
| E980a-h | S13 | L8 | M7 |
| E981a-h | S13 | L9 | M7 |
| E982a-h | S14 | L1 | M7 |
| E983a-h | S14 | L2 | M7 |
| E984a-h | S14 | L3 | M7 |
| E985a-h | S14 | L4 | M7 |
| E986a-h | S14 | L5 | M7 |
| E987a-h | S14 | L6 | M7 |
| E988a-h | S14 | L7 | M7 |
| E989a-h | S14 | L8 | M7 |
| E990a-h | S14 | L9 | M7 |
| E991a-h | S15 | L1 | M7 |
| E992a-h | S15 | L2 | M7 |
| E993a-h | S15 | L3 | M7 |
| E994a-h | S15 | L4 | M7 |
| E995a-h | S15 | L5 | M7 |
| E996a-h | S15 | L6 | M7 |
| E997a-h | S15 | L7 | M7 |
| E998a-h | S15 | L8 | M7 |
| E999a-h | S15 | L9 | M7 |
| E1000a-h | S16 | L1 | M7 |
| E1001a-h | S16 | L2 | M7 |
| E1002a-h | S16 | L3 | M7 |
| E1003a-h | S16 | L4 | M7 |
| E1004a-h | S16 | L5 | M7 |
| E1005a-h | S16 | L6 | M7 |
| E1006a-h | S16 | L7 | M7 |
| E1007a-h | S16 | L8 | M7 |
| E1008a-h | S16 | L9 | M7 |
| E1009a-h | S1 | L1 | M8 |
| E1010a-h | S1 | L2 | M8 |
| E1011a-h | S1 | L3 | M8 |
| E1012a-h | S1 | L4 | M8 |
| E1013a-h | S1 | L5 | M8 |
| E1014a-h | S1 | L6 | M8 |
| E1015a-h | S1 | L7 | M8 |
| E1016a-h | S1 | L8 | M8 |
| E1017a-h | S1 | L9 | M8 |
| E1018a-h | S2 | L1 | M8 |
| E1019a-h | S2 | L2 | M8 |
| E1020a-h | S2 | L3 | M8 |
| E1021a-h | S2 | L4 | M8 |
| E1022a-h | S2 | L5 | M8 |
| E1023a-h | S2 | L6 | M8 |
| E1024a-h | S2 | L7 | M8 |
| E1025a-h | S2 | L8 | M8 |
| E1026a-h | S2 | L9 | M8 |
| E1027a-h | S3 | L1 | M8 |
| E1028a-h | S3 | L2 | M8 |
| E1029a-h | S3 | L3 | M8 |
| E1030a-h | S3 | L4 | M8 |
| E1031a-h | S3 | L5 | M8 |
| E1032a-h | S3 | L6 | M8 |
| E1033a-h | S3 | L7 | M8 |
| E1034a-h | S3 | L8 | M8 |
| E1035a-h | S3 | L9 | M8 |
| E1036a-h | S4 | L1 | M8 |
| E1037a-h | S4 | L2 | M8 |
| E1038a-h | S4 | L3 | M8 |
| E1039a-h | S4 | L4 | M8 |
| E1040a-h | S4 | L5 | M8 |
| E1041a-h | S4 | L6 | M8 |
| E1042a-h | S4 | L7 | M8 |
| E1043a-h | S4 | L8 | M8 |
| E1044a-h | S4 | L9 | M8 |
| E1045a-h | S5 | L1 | M8 |
| E1046a-h | S5 | L2 | M8 |
| E1047a-h | S5 | L3 | M8 |
| E1048a-h | S5 | L4 | M8 |
| E1049a-h | S5 | L5 | M8 |
| E1050a-h | S5 | L6 | M8 |
| E1051a-h | S5 | L7 | M8 |
| E1052a-h | S5 | L8 | M8 |
| E1053a-h | S5 | L9 | M8 |
| E1054a-h | S6 | L1 | M8 |
| E1055a-h | S6 | L2 | M8 |
| E1056a-h | S6 | L3 | M8 |
| E1057a-h | S6 | L4 | M8 |
| E1058a-h | S6 | L5 | M8 |
| E1059a-h | S6 | L6 | M8 |
| E1060a-h | S6 | L7 | M8 |
| E1061a-h | S6 | L8 | M8 |
| E1062a-h | S6 | L9 | M8 |
| E1063a-h | S7 | L1 | M8 |
| E1064a-h | S7 | L2 | M8 |
| E1065a-h | S7 | L3 | M8 |
| E1066a-h | S7 | L4 | M8 |
| E1067a-h | S7 | L5 | M8 |
| E1068a-h | S7 | L6 | M8 |
| E1069a-h | S7 | L7 | M8 |
| E1070a-h | S7 | L8 | M8 |
| E1071a-h | S7 | L9 | M8 |
| E1072a-h | S8 | L1 | M8 |
| E1073a-h | S8 | L2 | M8 |
| E1074a-h | S8 | L3 | M8 |
| E1075a-h | S8 | L4 | M8 |
| E1076a-h | S8 | L5 | M8 |
| E1077a-h | S8 | L6 | M8 |
| E1078a-h | S8 | L7 | M8 |
| E1079a-h | S8 | L8 | M8 |
| E1080a-h | S8 | L9 | M8 |
| E1081a-h | S9 | L1 | M8 |
| E1082a-h | S9 | L2 | M8 |
| E1083a-h | S9 | L3 | M8 |
| E1084a-h | S9 | L4 | M8 |
| E1085a-h | S9 | L5 | M8 |
| E1086a-h | S9 | L6 | M8 |
| E1087a-h | S9 | L7 | M8 |
| E1088a-h | S9 | L8 | M8 |
| E1089a-h | S9 | L9 | M8 |
| E1090a-h | S10 | L1 | M8 |
| E1091a-h | S10 | L2 | M8 |
| E1092a-h | S10 | L3 | M8 |
| E1093a-h | S10 | L4 | M8 |
| E1094a-h | S10 | L5 | M8 |
| E1095a-h | S10 | L6 | M8 |
| E1096a-h | S10 | L7 | M8 |
| E1097a-h | S10 | L8 | M8 |
| E1098a-h | S10 | L9 | M8 |
| E1099a-h | S11 | L1 | M8 |
| E1100a-h | S11 | L2 | M8 |
| E1101a-h | S11 | L3 | M8 |
| E1102a-h | S11 | L4 | M8 |
| E1103a-h | S11 | L5 | M8 |
| E1104a-h | S11 | L6 | M8 |
| E1105a-h | S11 | L7 | M8 |
| E1106a-h | S11 | L8 | M8 |
| E1107a-h | S11 | L9 | M8 |
| E1108a-h | S12 | L1 | M8 |
| E1109a-h | S12 | L2 | M8 |
| E1110a-h | S12 | L3 | M8 |
| E1111a-h | S12 | L4 | M8 |
| E1112a-h | S12 | L5 | M8 |
| E1113a-h | S12 | L6 | M8 |
| E1114a-h | S12 | L7 | M8 |
| E1115a-h | S12 | L8 | M8 |
| E1116a-h | S12 | L9 | M8 |
| E1117a-h | S13 | L1 | M8 |
| E1118a-h | S13 | L2 | M8 |
| E1119a-h | S13 | L3 | M8 |
| E1120a-h | S13 | L4 | M8 |
| E1121a-h | S13 | L5 | M8 |
| E1122a-h | S13 | L6 | M8 |
| E1123a-h | S13 | L7 | M8 |
| E1124a-h | S13 | L8 | M8 |
| E1125a-h | S13 | L9 | M8 |
| E1126a-h | S14 | L1 | M8 |
| E1127a-h | S14 | L2 | M8 |
| E1128a-h | S14 | L3 | M8 |
| E1129a-h | S14 | L4 | M8 |
| E1130a-h | S14 | L5 | M8 |
| E1131a-h | S14 | L6 | M8 |
| E1132a-h | S14 | L7 | M8 |
| E1133a-h | S14 | L8 | M8 |
| E1134a-h | S14 | L9 | M8 |
| E1135a-h | S15 | L1 | M8 |
| E1136a-h | S15 | L2 | M8 |
| E1137a-h | S15 | L3 | M8 |
| E1138a-h | S15 | L4 | M8 |
| E1139a-h | S15 | L5 | M8 |
| E1140a-h | S15 | L6 | M8 |
| E1141a-h | S15 | L7 | M8 |
| E1142a-h | S15 | L8 | M8 |
| E1143a-h | S15 | L9 | M8 |
| E1144a-h | S16 | L1 | M8 |
| E1145a-h | S16 | L2 | M8 |
| E1146a-h | S16 | L3 | M8 |
| E1147a-h | S16 | L4 | M8 |
| E1148a-h | S16 | L5 | M8 |
| E1149a-h | S16 | L6 | M8 |
| E1150a-h | S16 | L7 | M8 |
| E1151a-h | S16 | L8 | M8 |
| E1152a-h | S16 | L9 | M8 |
| E1153a-h | S1 | L1 | M9 |
| E1154a-h | S1 | L2 | M9 |
| E1155a-h | S1 | L3 | M9 |
| E1156a-h | S1 | L4 | M9 |
| E1157a-h | S1 | L5 | M9 |
| E1158a-h | S1 | L6 | M9 |
| E1159a-h | S1 | L7 | M9 |
| E1160a-h | S1 | L8 | M9 |
| E1161a-h | S1 | L9 | M9 |
| E1162a-h | S2 | L1 | M9 |
| E1163a-h | S2 | L2 | M9 |
| E1164a-h | S2 | L3 | M9 |
| E1165a-h | S2 | L4 | M9 |
| E1166a-h | S2 | L5 | M9 |
| E1167a-h | S2 | L6 | M9 |
| E1168a-h | S2 | L7 | M9 |
| E1169a-h | S2 | L8 | M9 |
| E1170a-h | S2 | L9 | M9 |
| E1171a-h | S3 | L1 | M9 |
| E1172a-h | S3 | L2 | M9 |
| E1173a-h | S3 | L3 | M9 |
| E1174a-h | S3 | L4 | M9 |
| E1175a-h | S3 | L5 | M9 |
| E1176a-h | S3 | L6 | M9 |
| E1177a-h | S3 | L7 | M9 |
| E1178a-h | S3 | L8 | M9 |
| E1179a-h | S3 | L9 | M9 |
| E1180a-h | S4 | L1 | M9 |
| E1181a-h | S4 | L2 | M9 |
| E1182a-h | S4 | L3 | M9 |
| E1183a-h | S4 | L4 | M9 |
| E1184a-h | S4 | L5 | M9 |
| E1185a-h | S4 | L6 | M9 |
| E1186a-h | S4 | L7 | M9 |
| E1187a-h | S4 | L8 | M9 |
| E1188a-h | S4 | L9 | M9 |
| E1189a-h | S5 | L1 | M9 |
| E1190a-h | S5 | L2 | M9 |
| E1191a-h | S5 | L3 | M9 |
| E1192a-h | S5 | L4 | M9 |
| E1193a-h | S5 | L5 | M9 |
| E1194a-h | S5 | L6 | M9 |
| E1195a-h | S5 | L7 | M9 |
| E1196a-h | S5 | L8 | M9 |
| E1197a-h | S5 | L9 | M9 |
| E1198a-h | S6 | L1 | M9 |
| E1199a-h | S6 | L2 | M9 |
| E1200a-h | S6 | L3 | M9 |
| E1201a-h | S6 | L4 | M9 |
| E1202a-h | S6 | L5 | M9 |
| E1203a-h | S6 | L6 | M9 |
| E1204a-h | S6 | L7 | M9 |
| E1205a-h | S6 | L8 | M9 |
| E1206a-h | S6 | L9 | M9 |
| E1207a-h | S7 | L1 | M9 |
| E1208a-h | S7 | L2 | M9 |
| E1209a-h | S7 | L3 | M9 |
| E1210a-h | S7 | L4 | M9 |
| E1211a-h | S7 | L5 | M9 |
| E1212a-h | S7 | L6 | M9 |
| E1213a-h | S7 | L7 | M9 |
| E1214a-h | S7 | L8 | M9 |
| E1215a-h | S7 | L9 | M9 |
| E1216a-h | S8 | L1 | M9 |
| E1217a-h | S8 | L2 | M9 |
| E1218a-h | S8 | L3 | M9 |
| E1219a-h | S8 | L4 | M9 |
| E1220a-h | S8 | L5 | M9 |
| E1221a-h | S8 | L6 | M9 |
| E1222a-h | S8 | L7 | M9 |
| E1223a-h | S8 | L8 | M9 |
| E1224a-h | S8 | L9 | M9 |
| E1225a-h | S9 | L1 | M9 |
| E1226a-h | S9 | L2 | M9 |
| E1227a-h | S9 | L3 | M9 |
| E1228a-h | S9 | L4 | M9 |
| E1229a-h | S9 | L5 | M9 |
| E1230a-h | S9 | L6 | M9 |
| E1231a-h | S9 | L7 | M9 |
| E1232a-h | S9 | L8 | M9 |
| E1233a-h | S9 | L9 | M9 |
| E1234a-h | S10 | L1 | M9 |
| E1235a-h | S10 | L2 | M9 |
| E1236a-h | S10 | L3 | M9 |
| E1237a-h | S10 | L4 | M9 |
| E1238a-h | S10 | L5 | M9 |
| E1239a-h | S10 | L6 | M9 |
| E1240a-h | S10 | L7 | M9 |
| E1241a-h | S10 | L8 | M9 |
| E1242a-h | S10 | L9 | M9 |
| E1243a-h | S11 | L1 | M9 |
| E1244a-h | S11 | L2 | M9 |
| E1245a-h | S11 | L3 | M9 |
| E1246a-h | S11 | L4 | M9 |
| E1247a-h | S11 | L5 | M9 |
| E1248a-h | S11 | L6 | M9 |
| E1249a-h | S11 | L7 | M9 |
| E1250a-h | S11 | L8 | M9 |
| E1251a-h | S11 | L9 | M9 |
| E1252a-h | S12 | L1 | M9 |
| E1253a-h | S12 | L2 | M9 |
| E1254a-h | S12 | L3 | M9 |
| E1255a-h | S12 | L4 | M9 |
| E1256a-h | S12 | L5 | M9 |
| E1257a-h | S12 | L6 | M9 |
| E1258a-h | S12 | L7 | M9 |
| E1259a-h | S12 | L8 | M9 |
| E1260a-h | S12 | L9 | M9 |
| E1261a-h | S13 | L1 | M9 |
| E1262a-h | S13 | L2 | M9 |
| E1263a-h | S13 | L3 | M9 |
| E1264a-h | S13 | L4 | M9 |
| E1265a-h | S13 | L5 | M9 |
| E1266a-h | S13 | L6 | M9 |
| E1267a-h | S13 | L7 | M9 |
| E1268a-h | S13 | L8 | M9 |
| E1269a-h | S13 | L9 | M9 |
| E1270a-h | S14 | L1 | M9 |
| E1271a-h | S14 | L2 | M9 |
| E1272a-h | S14 | L3 | M9 |
| E1273a-h | S14 | L4 | M9 |
| E1274a-h | S14 | L5 | M9 |
| E1275a-h | S14 | L6 | M9 |
| E1276a-h | S14 | L7 | M9 |
| E1277a-h | S14 | L8 | M9 |
| E1278a-h | S14 | L9 | M9 |
| E1279a-h | S15 | L1 | M9 |
| E1280a-h | S15 | L2 | M9 |
| E1281a-h | S15 | L3 | M9 |
| E1282a-h | S15 | L4 | M9 |
| E1283a-h | S15 | L5 | M9 |
| E1284a-h | S15 | L6 | M9 |
| E1285a-h | S15 | L7 | M9 |
| E1286a-h | S15 | L8 | M9 |
| E1287a-h | S15 | L9 | M9 |
| E1288a-h | S16 | L1 | M9 |
| E1289a-h | S16 | L2 | M9 |
| E1290a-h | S16 | L3 | M9 |
| E1291a-h | S16 | L4 | M9 |
| E1292a-h | S16 | L5 | M9 |
| E1293a-h | S16 | L6 | M9 |
| E1294a-h | S16 | L7 | M9 |
| E1295a-h | S16 | L8 | M9 |
| E1296a-h | S16 | L9 | M9 |
| E1297a-h | S1 | L1 | M10 |
| E1298a-h | S1 | L2 | M10 |
| E1299a-h | S1 | L3 | M10 |
| E1300a-h | S1 | L4 | M10 |
| E1301a-h | S1 | L5 | M10 |
| E1302a-h | S1 | L6 | M10 |
| E1303a-h | S1 | L7 | M10 |
| E1304a-h | S1 | L8 | M10 |
| E1305a-h | S1 | L9 | M10 |
| E1306a-h | S2 | L1 | M10 |
| E1307a-h | S2 | L2 | M10 |
| E1308a-h | S2 | L3 | M10 |
| E1309a-h | S2 | L4 | M10 |
| E1310a-h | S2 | L5 | M10 |
| E1311a-h | S2 | L6 | M10 |
| E1312a-h | S2 | L7 | M10 |
| E1313a-h | S2 | L8 | M10 |
| E1314a-h | S2 | L9 | M10 |
| E1315a-h | S3 | L1 | M10 |
| E1316a-h | S3 | L2 | M10 |
| E1317a-h | S3 | L3 | M10 |
| E1318a-h | S3 | L4 | M10 |
| E1319a-h | S3 | L5 | M10 |
| E1320a-h | S3 | L6 | M10 |
| E1321a-h | S3 | L7 | M10 |
| E1322a-h | S3 | L8 | M10 |
| E1323a-h | S3 | L9 | M10 |
| E1324a-h | S4 | L1 | M10 |
| E1325a-h | S4 | L2 | M10 |
| E1326a-h | S4 | L3 | M10 |
| E1327a-h | S4 | L4 | M10 |
| E1328a-h | S4 | L5 | M10 |
| E1329a-h | S4 | L6 | M10 |
| E1330a-h | S4 | L7 | M10 |
| E1331a-h | S4 | L8 | M10 |
| E1332a-h | S4 | L9 | M10 |
| E1333a-h | S5 | L1 | M10 |
| E1334a-h | S5 | L2 | M10 |
| E1335a-h | S5 | L3 | M10 |
| E1336a-h | S5 | L4 | M10 |
| E1337a-h | S5 | L5 | M10 |
| E1338a-h | S5 | L6 | M10 |
| E1339a-h | S5 | L7 | M10 |
| E1340a-h | S5 | L8 | M10 |
| E1341a-h | S5 | L9 | M10 |
| E1342a-h | S6 | L1 | M10 |
| E1343a-h | S6 | L2 | M10 |
| E1344a-h | S6 | L3 | M10 |
| E1345a-h | S6 | L4 | M10 |
| E1346a-h | S6 | L5 | M10 |
| E1347a-h | S6 | L6 | M10 |
| E1348a-h | S6 | L7 | M10 |
| E1349a-h | S6 | L8 | M10 |
| E1350a-h | S6 | L9 | M10 |
| E1351a-h | S7 | L1 | M10 |
| E1352a-h | S7 | L2 | M10 |
| E1353a-h | S7 | L3 | M10 |
| E1354a-h | S7 | L4 | M10 |
| E1355a-h | S7 | L5 | M10 |
| E1356a-h | S7 | L6 | M10 |
| E1357a-h | S7 | L7 | M10 |
| E1358a-h | S7 | L8 | M10 |
| E1359a-h | S7 | L9 | M10 |
| E1360a-h | S8 | L1 | M10 |
| E1361a-h | S8 | L2 | M10 |
| E1362a-h | S8 | L3 | M10 |
| E1363a-h | S8 | L4 | M10 |
| E1364a-h | S8 | L5 | M10 |
| E1365a-h | S8 | L6 | M10 |
| E1366a-h | S8 | L7 | M10 |
| E1367a-h | S8 | L8 | M10 |
| E1368a-h | S8 | L9 | M10 |
| E1369a-h | S9 | L1 | M10 |
| E1370a-h | S9 | L2 | M10 |
| E1371a-h | S9 | L3 | M10 |
| E1372a-h | S9 | L4 | M10 |
| E1373a-h | S9 | L5 | M10 |
| E1374a-h | S9 | L6 | M10 |
| E1375a-h | S9 | L7 | M10 |
| E1376a-h | S9 | L8 | M10 |
| E1377a-h | S9 | L9 | M10 |
| E1378a-h | S10 | L1 | M10 |
| E1379a-h | S10 | L2 | M10 |
| E1380a-h | S10 | L3 | M10 |
| E1381a-h | S10 | L4 | M10 |
| E1382a-h | S10 | L5 | M10 |
| E1383a-h | S10 | L6 | M10 |
| E1384a-h | S10 | L7 | M10 |
| E1385a-h | S10 | L8 | M10 |
| E1386a-h | S10 | L9 | M10 |
| E1387a-h | S11 | L1 | M10 |
| E1388a-h | S11 | L2 | M10 |
| E1389a-h | S11 | L3 | M10 |
| E1390a-h | S11 | L4 | M10 |
| E1391a-h | S11 | L5 | M10 |
| E1392a-h | S11 | L6 | M10 |
| E1393a-h | S11 | L7 | M10 |
| E1394a-h | S11 | L8 | M10 |
| E1395a-h | S11 | L9 | M10 |
| E1396a-h | S12 | L1 | M10 |
| E1397a-h | S12 | L2 | M10 |
| E1398a-h | S12 | L3 | M10 |
| E1399a-h | S12 | L4 | M10 |
| E1400a-h | S12 | L5 | M10 |
| E1401a-h | S12 | L6 | M10 |
| E1402a-h | S12 | L7 | M10 |
| E1403a-h | S12 | L8 | M10 |
| E1404a-h | S12 | L9 | M10 |
| E1405a-h | S13 | L1 | M10 |
| E1406a-h | S13 | L2 | M10 |
| E1407a-h | S13 | L3 | M10 |
| E1408a-h | S13 | L4 | M10 |
| E1409a-h | S13 | L5 | M10 |
| E1410a-h | S13 | L6 | M10 |
| E1411a-h | S13 | L7 | M10 |
| E1412a-h | S13 | L8 | M10 |
| E1413a-h | S13 | L9 | M10 |
| E1414a-h | S14 | L1 | M10 |
| E1415a-h | S14 | L2 | M10 |
| E1416a-h | S14 | L3 | M10 |
| E1417a-h | S14 | L4 | M10 |
| E1418a-h | S14 | L5 | M10 |
| E1419a-h | S14 | L6 | M10 |
| E1420a-h | S14 | L7 | M10 |
| E1421a-h | S14 | L8 | M10 |
| E1422a-h | S14 | L9 | M10 |
| E1423a-h | S15 | L1 | M10 |
| E1424a-h | S15 | L2 | M10 |
| E1425a-h | S15 | L3 | M10 |
| E1426a-h | S15 | L4 | M10 |
| E1427a-h | S15 | L5 | M10 |
| E1428a-h | S15 | L6 | M10 |
| E1429a-h | S15 | L7 | M10 |
| E1430a-h | S15 | L8 | M10 |
| E1431a-h | S15 | L9 | M10 |
| E1432a-h | S16 | L1 | M10 |
| E1433a-h | S16 | L2 | M10 |
| E1434a-h | S16 | L3 | M10 |
| E1435a-h | S16 | L4 | M10 |
| E1436a-h | S16 | L5 | M10 |
| E1437a-h | S16 | L6 | M10 |
| E1438a-h | S16 | L7 | M10 |
| E1439a-h | S16 | L8 | M10 |
| E1440a-h | S16 | L9 | M10 |
| E1441a-h | S1 | L1 | M11 |
| E1442a-h | S1 | L2 | M11 |
| E1443a-h | S1 | L3 | M11 |
| E1444a-h | S1 | L4 | M11 |
| E1445a-h | S1 | L5 | M11 |
| E1446a-h | S1 | L6 | M11 |
| E1447a-h | S1 | L7 | M11 |
| E1448a-h | S1 | L8 | M11 |
| E1449a-h | S1 | L9 | M11 |
| E1450a-h | S2 | L1 | M11 |
| E1451a-h | S2 | L2 | M11 |
| E1452a-h | S2 | L3 | M11 |
| E1453a-h | S2 | L4 | M11 |
| E1454a-h | S2 | L5 | M11 |
| E1455a-h | S2 | L6 | M11 |
| E1456a-h | S2 | L7 | M11 |
| E1457a-h | S2 | L8 | M11 |
| E1458a-h | S2 | L9 | M11 |
| E1459a-h | S3 | L1 | M11 |
| E1460a-h | S3 | L2 | M11 |
| E1461a-h | S3 | L3 | M11 |
| E1462a-h | S3 | L4 | M11 |
| E1463a-h | S3 | L5 | M11 |
| E1464a-h | S3 | L6 | M11 |
| E1465a-h | S3 | L7 | M11 |
| E1466a-h | S3 | L8 | M11 |
| E1467a-h | S3 | L9 | M11 |
| E1468a-h | S4 | L1 | M11 |
| E1469a-h | S4 | L2 | M11 |
| E1470a-h | S4 | L3 | M11 |
| E1471a-h | S4 | L4 | M11 |
| E1472a-h | S4 | L5 | M11 |
| E1473a-h | S4 | L6 | M11 |
| E1474a-h | S4 | L7 | M11 |
| E1475a-h | S4 | L8 | M11 |
| E1476a-h | S4 | L9 | M11 |
| E1477a-h | S5 | L1 | M11 |
| E1478a-h | S5 | L2 | M11 |
| E1479a-h | S5 | L3 | M11 |
| E1480a-h | S5 | L4 | M11 |
| E1481a-h | S5 | L5 | M11 |
| E1482a-h | S5 | L6 | M11 |
| E1483a-h | S5 | L7 | M11 |
| E1484a-h | S5 | L8 | M11 |
| E1485a-h | S5 | L9 | M11 |
| E1486a-h | S6 | L1 | M11 |
| E1487a-h | S6 | L2 | M11 |
| E1488a-h | S6 | L3 | M11 |
| E1489a-h | S6 | L4 | M11 |
| E1490a-h | S6 | L5 | M11 |
| E1491a-h | S6 | L6 | M11 |
| E1492a-h | S6 | L7 | M11 |
| E1493a-h | S6 | L8 | M11 |
| E1494a-h | S6 | L9 | M11 |
| E1495a-h | S7 | L1 | M11 |
| E1496a-h | S7 | L2 | M11 |
| E1497a-h | S7 | L3 | M11 |
| E1498a-h | S7 | L4 | M11 |
| E1499a-h | S7 | L5 | M11 |
| E1500a-h | S7 | L6 | M11 |
| E1501a-h | S7 | L7 | M11 |
| E1502a-h | S7 | L8 | M11 |
| E1503a-h | S7 | L9 | M11 |
| E1504a-h | S8 | L1 | M11 |
| E1505a-h | S8 | L2 | M11 |
| E1506a-h | S8 | L3 | M11 |
| E1507a-h | S8 | L4 | M11 |
| E1508a-h | S8 | L5 | M11 |
| E1509a-h | S8 | L6 | M11 |
| E1510a-h | S8 | L7 | M11 |
| E1511a-h | S8 | L8 | M11 |
| E1512a-h | S8 | L9 | M11 |
| E1513a-h | S9 | L1 | M11 |
| E1514a-h | S9 | L2 | M11 |
| E1515a-h | S9 | L3 | M11 |
| E1516a-h | S9 | L4 | M11 |
| E1517a-h | S9 | L5 | M11 |
| E1518a-h | S9 | L6 | M11 |
| E1519a-h | S9 | L7 | M11 |
| E1520a-h | S9 | L8 | M11 |
| E1521a-h | S9 | L9 | M11 |
| E1522a-h | S10 | L1 | M11 |
| E1523a-h | S10 | L2 | M11 |
| E1524a-h | S10 | L3 | M11 |
| E1525a-h | S10 | L4 | M11 |
| E1526a-h | S10 | L5 | M11 |
| E1527a-h | S10 | L6 | M11 |
| E1528a-h | S10 | L7 | M11 |
| E1529a-h | S10 | L8 | M11 |
| E1530a-h | S10 | L9 | M11 |
| E1531a-h | S11 | L1 | M11 |
| E1532a-h | S11 | L2 | M11 |
| E1533a-h | S11 | L3 | M11 |
| E1534a-h | S11 | L4 | M11 |
| E1535a-h | S11 | L5 | M11 |
| E1536a-h | S11 | L6 | M11 |
| E1537a-h | S11 | L7 | M11 |
| E1538a-h | S11 | L8 | M11 |
| E1539a-h | S11 | L9 | M11 |
| E1540a-h | S12 | L1 | M11 |
| E1541a-h | S12 | L2 | M11 |
| E1542a-h | S12 | L3 | M11 |
| E1543a-h | S12 | L4 | M11 |
| E1544a-h | S12 | L5 | M11 |
| E1545a-h | S12 | L6 | M11 |
| E1546a-h | S12 | L7 | M11 |
| E1547a-h | S12 | L8 | M11 |
| E1548a-h | S12 | L9 | M11 |
| E1549a-h | S13 | L1 | M11 |
| E1550a-h | S13 | L2 | M11 |
| E1551a-h | S13 | L3 | M11 |
| E1552a-h | S13 | L4 | M11 |
| E1553a-h | S13 | L5 | M11 |
| E1554a-h | S13 | L6 | M11 |
| E1555a-h | S13 | L7 | M11 |
| E1556a-h | S13 | L8 | M11 |
| E1557a-h | S13 | L9 | M11 |
| E1558a-h | S14 | L1 | M11 |
| E1559a-h | S14 | L2 | M11 |
| E1560a-h | S14 | L3 | M11 |
| E1561a-h | S14 | L4 | M11 |
| E1562a-h | S14 | L5 | M11 |
| E1563a-h | S14 | L6 | M11 |
| E1564a-h | S14 | L7 | M11 |
| E1565a-h | S14 | L8 | M11 |
| E1566a-h | S14 | L9 | M11 |
| E1567a-h | S15 | L1 | M11 |
| E1568a-h | S15 | L2 | M11 |
| E1569a-h | S15 | L3 | M11 |
| E1570a-h | S15 | L4 | M11 |
| E1571a-h | S15 | L5 | M11 |
| E1572a-h | S15 | L6 | M11 |
| E1573a-h | S15 | L7 | M11 |
| E1574a-h | S15 | L8 | M11 |
| E1575a-h | S15 | L9 | M11 |
| E1576a-h | S16 | L1 | M11 |
| E1577a-h | S16 | L2 | M11 |
| E1578a-h | S16 | L3 | M11 |
| E1579a-h | S16 | L4 | M11 |
| E1580a-h | S16 | L5 | M11 |
| E1581a-h | S16 | L6 | M11 |
| E1582a-h | S16 | L7 | M11 |
| E1583a-h | S16 | L8 | M11 |
| E1584a-h | S16 | L9 | M11 |
| E1585a-h | S1 | L1 | M12 |
| E1586a-h | S1 | L2 | M12 |
| E1587a-h | S1 | L3 | M12 |
| E1588a-h | S1 | L4 | M12 |
| E1589a-h | S1 | L5 | M12 |
| E1590a-h | S1 | L6 | M12 |
| E1591a-h | S1 | L7 | M12 |
| E1592a-h | S1 | L8 | M12 |
| E1593a-h | S1 | L9 | M12 |
| E1594a-h | S2 | L1 | M12 |
| E1595a-h | S2 | L2 | M12 |
| E1596a-h | S2 | L3 | M12 |
| E1597a-h | S2 | L4 | M12 |
| E1598a-h | S2 | L5 | M12 |
| E1599a-h | S2 | L6 | M12 |
| E1600a-h | S2 | L7 | M12 |
| E1601a-h | S2 | L8 | M12 |
| E1602a-h | S2 | L9 | M12 |
| E1603a-h | S3 | L1 | M12 |
| E1604a-h | S3 | L2 | M12 |
| E1605a-h | S3 | L3 | M12 |
| E1606a-h | S3 | L4 | M12 |
| E1607a-h | S3 | L5 | M12 |
| E1608a-h | S3 | L6 | M12 |
| E1609a-h | S3 | L7 | M12 |
| E1610a-h | S3 | L8 | M12 |
| E1611a-h | S3 | L9 | M12 |
| E1612a-h | S4 | L1 | M12 |
| E1613a-h | S4 | L2 | M12 |
| E1614a-h | S4 | L3 | M12 |
| E1615a-h | S4 | L4 | M12 |
| E1616a-h | S4 | L5 | M12 |
| E1617a-h | S4 | L6 | M12 |
| E1618a-h | S4 | L7 | M12 |
| E1619a-h | S4 | L8 | M12 |
| E1620a-h | S4 | L9 | M12 |
| E1621a-h | S5 | L1 | M12 |
| E1622a-h | S5 | L2 | M12 |
| E1623a-h | S5 | L3 | M12 |
| E1624a-h | S5 | L4 | M12 |
| E1625a-h | S5 | L5 | M12 |
| E1626a-h | S5 | L6 | M12 |
| E1627a-h | S5 | L7 | M12 |
| E1628a-h | S5 | L8 | M12 |
| E1629a-h | S5 | L9 | M12 |
| E1630a-h | S6 | L1 | M12 |
| E1631a-h | S6 | L2 | M12 |
| E1632a-h | S6 | L3 | M12 |
| E1633a-h | S6 | L4 | M12 |
| E1634a-h | S6 | L5 | M12 |
| E1635a-h | S6 | L6 | M12 |
| E1636a-h | S6 | L7 | M12 |
| E1637a-h | S6 | L8 | M12 |
| E1638a-h | S6 | L9 | M12 |
| E1639a-h | S7 | L1 | M12 |
| E1640a-h | S7 | L2 | M12 |
| E1641a-h | S7 | L3 | M12 |
| E1642a-h | S7 | L4 | M12 |
| E1643a-h | S7 | L5 | M12 |
| E1644a-h | S7 | L6 | M12 |
| E1645a-h | S7 | L7 | M12 |
| E1646a-h | S7 | L8 | M12 |
| E1647a-h | S7 | L9 | M12 |
| E1648a-h | S8 | L1 | M12 |
| E1649a-h | S8 | L2 | M12 |
| E1650a-h | S8 | L3 | M12 |
| E1651a-h | S8 | L4 | M12 |
| E1652a-h | S8 | L5 | M12 |
| E1653a-h | S8 | L6 | M12 |
| E1654a-h | S8 | L7 | M12 |
| E1655a-h | S8 | L8 | M12 |
| E1656a-h | S8 | L9 | M12 |
| E1657a-h | S9 | L1 | M12 |
| E1658a-h | S9 | L2 | M12 |
| E1659a-h | S9 | L3 | M12 |
| E1660a-h | S9 | L4 | M12 |
| E1661a-h | S9 | L5 | M12 |
| E1662a-h | S9 | L6 | M12 |
| E1663a-h | S9 | L7 | M12 |
| E1664a-h | S9 | L8 | M12 |
| E1665a-h | S9 | L9 | M12 |
| E1666a-h | S10 | L1 | M12 |
| E1667a-h | S10 | L2 | M12 |
| E1668a-h | S10 | L3 | M12 |
| E1669a-h | S10 | L4 | M12 |
| E1670a-h | S10 | L5 | M12 |
| E1671a-h | S10 | L6 | M12 |
| E1672a-h | S10 | L7 | M12 |
| E1673a-h | S10 | L8 | M12 |
| E1674a-h | S10 | L9 | M12 |
| E1675a-h | S11 | L1 | M12 |
| E1676a-h | S11 | L2 | M12 |
| E1677a-h | S11 | L3 | M12 |
| E1678a-h | S11 | L4 | M12 |
| E1679a-h | S11 | L5 | M12 |
| E1680a-h | S11 | L6 | M12 |
| E1681a-h | S11 | L7 | M12 |
| E1682a-h | S11 | L8 | M12 |
| E1683a-h | S11 | L9 | M12 |
| E1684a-h | S12 | L1 | M12 |
| E1685a-h | S12 | L2 | M12 |
| E1686a-h | S12 | L3 | M12 |
| E1687a-h | S12 | L4 | M12 |
| E1688a-h | S12 | L5 | M12 |
| E1689a-h | S12 | L6 | M12 |
| E1690a-h | S12 | L7 | M12 |
| E1691a-h | S12 | L8 | M12 |
| E1692a-h | S12 | L9 | M12 |
| E1693a-h | S13 | L1 | M12 |
| E1694a-h | S13 | L2 | M12 |
| E1695a-h | S13 | L3 | M12 |
| E1696a-h | S13 | L4 | M12 |
| E1697a-h | S13 | L5 | M12 |
| E1698a-h | S13 | L6 | M12 |
| E1699a-h | S13 | L7 | M12 |
| E1700a-h | S13 | L8 | M12 |
| E1701a-h | S13 | L9 | M12 |
| E1702a-h | S14 | L1 | M12 |
| E1703a-h | S14 | L2 | M12 |
| E1704a-h | S14 | L3 | M12 |
| E1705a-h | S14 | L4 | M12 |
| E1706a-h | S14 | L5 | M12 |
| E1707a-h | S14 | L6 | M12 |
| E1708a-h | S14 | L7 | M12 |
| E1709a-h | S14 | L8 | M12 |
| E1710a-h | S14 | L9 | M12 |
| E1711a-h | S15 | L1 | M12 |
| E1712a-h | S15 | L2 | M12 |
| E1713a-h | S15 | L3 | M12 |
| E1714a-h | S15 | L4 | M12 |
| E1715a-h | S15 | L5 | M12 |
| E1716a-h | S15 | L6 | M12 |
| E1717a-h | S15 | L7 | M12 |
| E1718a-h | S15 | L8 | M12 |
| E1719a-h | S15 | L9 | M12 |
| E1720a-h | S16 | L1 | M12 |
| E1721a-h | S16 | L2 | M12 |
| E1722a-h | S16 | L3 | M12 |
| E1723a-h | S16 | L4 | M12 |
| E1724a-h | S16 | L5 | M12 |
| E1725a-h | S16 | L6 | M12 |
| E1726a-h | S16 | L7 | M12 |
| E1727a-h | S16 | L8 | M12 |
| E1728a-h | S16 | L9 | M12 |
| E1729a-h | S1 | L1 | M13 |
| E1730a-h | S1 | L2 | M13 |
| E1731a-h | S1 | L3 | M13 |
| E1732a-h | S1 | L4 | M13 |
| E1733a-h | S1 | L5 | M13 |
| E1734a-h | S1 | L6 | M13 |
| E1735a-h | S1 | L7 | M13 |
| E1736a-h | S1 | L8 | M13 |
| E1737a-h | S1 | L9 | M13 |
| E1738a-h | S2 | L1 | M13 |
| E1739a-h | S2 | L2 | M13 |
| E1740a-h | S2 | L3 | M13 |
| E1741a-h | S2 | L4 | M13 |
| E1742a-h | S2 | L5 | M13 |
| E1743a-h | S2 | L6 | M13 |
| E1744a-h | S2 | L7 | M13 |
| E1745a-h | S2 | L8 | M13 |
| E1746a-h | S2 | L9 | M13 |
| E1747a-h | S3 | L1 | M13 |
| E1748a-h | S3 | L2 | M13 |
| E1749a-h | S3 | L3 | M13 |
| E1750a-h | S3 | L4 | M13 |
| E1751a-h | S3 | L5 | M13 |
| E1752a-h | S3 | L6 | M13 |
| E1753a-h | S3 | L7 | M13 |
| E1754a-h | S3 | L8 | M13 |
| E1755a-h | S3 | L9 | M13 |
| E1756a-h | S4 | L1 | M13 |
| E1757a-h | S4 | L2 | M13 |
| E1758a-h | S4 | L3 | M13 |
| E1759a-h | S4 | L4 | M13 |
| E1760a-h | S4 | L5 | M13 |
| E1761a-h | S4 | L6 | M13 |
| E1762a-h | S4 | L7 | M13 |
| E1763a-h | S4 | L8 | M13 |
| E1764a-h | S4 | L9 | M13 |
| E1765a-h | S5 | L1 | M13 |
| E1766a-h | S5 | L2 | M13 |
| E1767a-h | S5 | L3 | M13 |
| E1768a-h | S5 | L4 | M13 |
| E1769a-h | S5 | L5 | M13 |
| E1770a-h | S5 | L6 | M13 |
| E1771a-h | S5 | L7 | M13 |
| E1772a-h | S5 | L8 | M13 |
| E1773a-h | S5 | L9 | M13 |
| E1774a-h | S6 | L1 | M13 |
| E1775a-h | S6 | L2 | M13 |
| E1776a-h | S6 | L3 | M13 |
| E1777a-h | S6 | L4 | M13 |
| E1778a-h | S6 | L5 | M13 |
| E1779a-h | S6 | L6 | M13 |
| E1780a-h | S6 | L7 | M13 |
| E1781a-h | S6 | L8 | M13 |
| E1782a-h | S6 | L9 | M13 |
| E1783a-h | S7 | L1 | M13 |
| E1784a-h | S7 | L2 | M13 |
| E1785a-h | S7 | L3 | M13 |
| E1786a-h | S7 | L4 | M13 |
| E1787a-h | S7 | L5 | M13 |
| E1788a-h | S7 | L6 | M13 |
| E1789a-h | S7 | L7 | M13 |
| E1790a-h | S7 | L8 | M13 |
| E1791a-h | S7 | L9 | M13 |
| E1792a-h | S8 | L1 | M13 |
| E1793a-h | S8 | L2 | M13 |
| E1794a-h | S8 | L3 | M13 |
| E1795a-h | S8 | L4 | M13 |
| E1796a-h | S8 | L5 | M13 |
| E1797a-h | S8 | L6 | M13 |
| E1798a-h | S8 | L7 | M13 |
| E1799a-h | S8 | L8 | M13 |
| E1800a-h | S8 | L9 | M13 |
| E1801a-h | S9 | L1 | M13 |
| E1802a-h | S9 | L2 | M13 |
| E1803a-h | S9 | L3 | M13 |
| E1804a-h | S9 | L4 | M13 |
| E1805a-h | S9 | L5 | M13 |
| E1806a-h | S9 | L6 | M13 |
| E1807a-h | S9 | L7 | M13 |
| E1808a-h | S9 | L8 | M13 |
| E1809a-h | S9 | L9 | M13 |
| E1810a-h | S10 | L1 | M13 |
| E1811a-h | S10 | L2 | M13 |
| E1812a-h | S10 | L3 | M13 |
| E1813a-h | S10 | L4 | M13 |
| E1814a-h | S10 | L5 | M13 |
| E1815a-h | S10 | L6 | M13 |
| E1816a-h | S10 | L7 | M13 |
| E1817a-h | S10 | L8 | M13 |
| E1818a-h | S10 | L9 | M13 |
| E1819a-h | S11 | L1 | M13 |
| E1820a-h | S11 | L2 | M13 |
| E1821a-h | S11 | L3 | M13 |
| E1822a-h | S11 | L4 | M13 |
| E1823a-h | S11 | L5 | M13 |
| E1824a-h | S11 | L6 | M13 |
| E1825a-h | S11 | L7 | M13 |
| E1826a-h | S11 | L8 | M13 |
| E1827a-h | S11 | L9 | M13 |
| E1828a-h | S12 | L1 | M13 |
| E1829a-h | S12 | L2 | M13 |
| E1830a-h | S12 | L3 | M13 |
| E1831a-h | S12 | L4 | M13 |
| E1832a-h | S12 | L5 | M13 |
| E1833a-h | S12 | L6 | M13 |
| E1834a-h | S12 | L7 | M13 |
| E1835a-h | S12 | L8 | M13 |
| E1836a-h | S12 | L9 | M13 |
| E1837a-h | S13 | L1 | M13 |
| E1838a-h | S13 | L2 | M13 |
| E1839a-h | S13 | L3 | M13 |
| E1840a-h | S13 | L4 | M13 |
| E1841a-h | S13 | L5 | M13 |
| E1842a-h | S13 | L6 | M13 |
| E1843a-h | S13 | L7 | M13 |
| E1844a-h | S13 | L8 | M13 |
| E1845a-h | S13 | L9 | M13 |
| E1846a-h | S14 | L1 | M13 |
| E1847a-h | S14 | L2 | M13 |
| E1848a-h | S14 | L3 | M13 |
| E1849a-h | S14 | L4 | M13 |
| E1850a-h | S14 | L5 | M13 |
| E1851a-h | S14 | L6 | M13 |
| E1852a-h | S14 | L7 | M13 |
| E1853a-h | S14 | L8 | M13 |
| E1854a-h | S14 | L9 | M13 |
| E1855a-h | S15 | L1 | M13 |
| E1856a-h | S15 | L2 | M13 |
| E1857a-h | S15 | L3 | M13 |
| E1858a-h | S15 | L4 | M13 |
| E1859a-h | S15 | L5 | M13 |
| E1860a-h | S15 | L6 | M13 |
| E1861a-h | S15 | L7 | M13 |
| E1862a-h | S15 | L8 | M13 |
| E1863a-h | S15 | L9 | M13 |
| E1864a-h | S16 | L1 | M13 |
| E1865a-h | S16 | L2 | M13 |
| E1866a-h | S16 | L3 | M13 |
| E1867a-h | S16 | L4 | M13 |
| E1868a-h | S16 | L5 | M13 |
| E1869a-h | S16 | L6 | M13 |
| E1870a-h | S16 | L7 | M13 |
| E1871a-h | S16 | L8 | M13 |
| E1872a-h | S16 | L9 | M13 |
| E1873a-h | S1 | L1 | M14 |
| E1874a-h | S1 | L2 | M14 |
| E1875a-h | S1 | L3 | M14 |
| E1876a-h | S1 | L4 | M14 |
| E1877a-h | S1 | L5 | M14 |
| E1878a-h | S1 | L6 | M14 |
| E1879a-h | S1 | L7 | M14 |
| E1880a-h | S1 | L8 | M14 |
| E1881a-h | S1 | L9 | M14 |
| E1882a-h | S2 | L1 | M14 |
| E1883a-h | S2 | L2 | M14 |
| E1884a-h | S2 | L3 | M14 |
| E1885a-h | S2 | L4 | M14 |
| E1886a-h | S2 | L5 | M14 |
| E1887a-h | S2 | L6 | M14 |
| E1888a-h | S2 | L7 | M14 |
| E1889a-h | S2 | L8 | M14 |
| E1890a-h | S2 | L9 | M14 |
| E1891a-h | S3 | L1 | M14 |
| E1892a-h | S3 | L2 | M14 |
| E1893a-h | S3 | L3 | M14 |
| E1894a-h | S3 | L4 | M14 |
| E1895a-h | S3 | L5 | M14 |
| E1896a-h | S3 | L6 | M14 |
| E1897a-h | S3 | L7 | M14 |
| E1898a-h | S3 | L8 | M14 |
| E1899a-h | S3 | L9 | M14 |
| E1900a-h | S4 | L1 | M14 |
| E1901a-h | S4 | L2 | M14 |
| E1902a-h | S4 | L3 | M14 |
| E1903a-h | S4 | L4 | M14 |
| E1904a-h | S4 | L5 | M14 |
| E1905a-h | S4 | L6 | M14 |
| E1906a-h | S4 | L7 | M14 |
| E1907a-h | S4 | L8 | M14 |
| E1908a-h | S4 | L9 | M14 |
| E1909a-h | S5 | L1 | M14 |
| E1910a-h | S5 | L2 | M14 |
| E1911a-h | S5 | L3 | M14 |
| E1912a-h | S5 | L4 | M14 |
| E1913a-h | S5 | L5 | M14 |
| E1914a-h | S5 | L6 | M14 |
| E1915a-h | S5 | L7 | M14 |
| E1916a-h | S5 | L8 | M14 |
| E1917a-h | S5 | L9 | M14 |
| E1918a-h | S6 | L1 | M14 |
| E1919a-h | S6 | L2 | M14 |
| E1920a-h | S6 | L3 | M14 |
| E1921a-h | S6 | L4 | M14 |
| E1922a-h | S6 | L5 | M14 |
| E1923a-h | S6 | L6 | M14 |
| E1924a-h | S6 | L7 | M14 |
| E1925a-h | S6 | L8 | M14 |
| E1926a-h | S6 | L9 | M14 |
| E1927a-h | S7 | L1 | M14 |
| E1928a-h | S7 | L2 | M14 |
| E1929a-h | S7 | L3 | M14 |
| E1930a-h | S7 | L4 | M14 |
| E1931a-h | S7 | L5 | M14 |
| E1932a-h | S7 | L6 | M14 |
| E1933a-h | S7 | L7 | M14 |
| E1934a-h | S7 | L8 | M14 |
| E1935a-h | S7 | L9 | M14 |
| E1936a-h | S8 | L1 | M14 |
| E1937a-h | S8 | L2 | M14 |
| E1938a-h | S8 | L3 | M14 |
| E1939a-h | S8 | L4 | M14 |
| E1940a-h | S8 | L5 | M14 |
| E1941a-h | S8 | L6 | M14 |
| E1942a-h | S8 | L7 | M14 |
| E1943a-h | S8 | L8 | M14 |
| E1944a-h | S8 | L9 | M14 |
| E1945a-h | S9 | L1 | M14 |
| E1946a-h | S9 | L2 | M14 |
| E1947a-h | S9 | L3 | M14 |
| E1948a-h | S9 | L4 | M14 |
| E1949a-h | S9 | L5 | M14 |
| E1950a-h | S9 | L6 | M14 |
| E1951a-h | S9 | L7 | M14 |
| E1952a-h | S9 | L8 | M14 |
| E1953a-h | S9 | L9 | M14 |
| E1954a-h | S10 | L1 | M14 |
| E1955a-h | S10 | L2 | M14 |
| E1956a-h | S10 | L3 | M14 |
| E1957a-h | S10 | L4 | M14 |
| E1958a-h | S10 | L5 | M14 |
| E1959a-h | S10 | L6 | M14 |
| E1960a-h | S10 | L7 | M14 |
| E1961a-h | S10 | L8 | M14 |
| E1962a-h | S10 | L9 | M14 |
| E1963a-h | S11 | L1 | M14 |
| E1964a-h | S11 | L2 | M14 |
| E1965a-h | S11 | L3 | M14 |
| E1966a-h | S11 | L4 | M14 |
| E1967a-h | S11 | L5 | M14 |
| E1968a-h | S11 | L6 | M14 |
| E1969a-h | S11 | L7 | M14 |
| E1970a-h | S11 | L8 | M14 |
| E1971a-h | S11 | L9 | M14 |
| E1972a-h | S12 | L1 | M14 |
| E1973a-h | S12 | L2 | M14 |
| E1974a-h | S12 | L3 | M14 |
| E1975a-h | S12 | L4 | M14 |
| E1976a-h | S12 | L5 | M14 |
| E1977a-h | S12 | L6 | M14 |
| E1978a-h | S12 | L7 | M14 |
| E1979a-h | S12 | L8 | M14 |
| E1980a-h | S12 | L9 | M14 |
| E1981a-h | S13 | L1 | M14 |
| E1982a-h | S13 | L2 | M14 |
| E1983a-h | S13 | L3 | M14 |
| E1984a-h | S13 | L4 | M14 |
| E1985a-h | S13 | L5 | M14 |
| E1986a-h | S13 | L6 | M14 |
| E1987a-h | S13 | L7 | M14 |
| E1988a-h | S13 | L8 | M14 |
| E1989a-h | S13 | L9 | M14 |
| E1990a-h | S14 | L1 | M14 |
| E1991a-h | S14 | L2 | M14 |
| E1992a-h | S14 | L3 | M14 |
| E1993a-h | S14 | L4 | M14 |
| E1994a-h | S14 | L5 | M14 |
| E1995a-h | S14 | L6 | M14 |
| E1996a-h | S14 | L7 | M14 |
| E1997a-h | S14 | L8 | M14 |
| E1998a-h | S14 | L9 | M14 |
| E1999a-h | S15 | L1 | M14 |
| E2000a-h | S15 | L2 | M14 |
| E2001a-h | S15 | L3 | M14 |
| E2002a-h | S15 | L4 | M14 |
| E2003a-h | S15 | L5 | M14 |
| E2004a-h | S15 | L6 | M14 |
| E2005a-h | S15 | L7 | M14 |
| E2006a-h | S15 | L8 | M14 |
| E2007a-h | S15 | L9 | M14 |
| E2008a-h | S16 | L1 | M14 |
| E2009a-h | S16 | L2 | M14 |
| E2010a-h | S16 | L3 | M14 |
| E2011a-h | S16 | L4 | M14 |
| E2012a-h | S16 | L5 | M14 |
| E2013a-h | S16 | L6 | M14 |
| E2014a-h | S16 | L7 | M14 |
| E2015a-h | S16 | L8 | M14 |
| E2016a-h | S16 | L9 | M14 |
| E2017a-h | S1 | L1 | M15 |
| E2018a-h | S1 | L2 | M15 |
| E2019a-h | S1 | L3 | M15 |
| E2020a-h | S1 | L4 | M15 |
| E2021a-h | S1 | L5 | M15 |
| E2022a-h | S1 | L6 | M15 |
| E2023a-h | S1 | L7 | M15 |
| E2024a-h | S1 | L8 | M15 |
| E2025a-h | S1 | L9 | M15 |
| E2026a-h | S2 | L1 | M15 |
| E2027a-h | S2 | L2 | M15 |
| E2028a-h | S2 | L3 | M15 |
| E2029a-h | S2 | L4 | M15 |
| E2030a-h | S2 | L5 | M15 |
| E2031a-h | S2 | L6 | M15 |
| E2032a-h | S2 | L7 | M15 |
| E2033a-h | S2 | L8 | M15 |
| E2034a-h | S2 | L9 | M15 |
| E2035a-h | S3 | L1 | M15 |
| E2036a-h | S3 | L2 | M15 |
| E2037a-h | S3 | L3 | M15 |
| E2038a-h | S3 | L4 | M15 |
| E2039a-h | S3 | L5 | M15 |
| E2040a-h | S3 | L6 | M15 |
| E2041a-h | S3 | L7 | M15 |
| E2042a-h | S3 | L8 | M15 |
| E2043a-h | S3 | L9 | M15 |
| E2044a-h | S4 | L1 | M15 |
| E2045a-h | S4 | L2 | M15 |
| E2046a-h | S4 | L3 | M15 |
| E2047a-h | S4 | L4 | M15 |
| E2048a-h | S4 | L5 | M15 |
| E2049a-h | S4 | L6 | M15 |
| E2050a-h | S4 | L7 | M15 |
| E2051a-h | S4 | L8 | M15 |
| E2052a-h | S4 | L9 | M15 |
| E2053a-h | S5 | L1 | M15 |
| E2054a-h | S5 | L2 | M15 |
| E2055a-h | S5 | L3 | M15 |
| E2056a-h | S5 | L4 | M15 |
| E2057a-h | S5 | L5 | M15 |
| E2058a-h | S5 | L6 | M15 |
| E2059a-h | S5 | L7 | M15 |
| E2060a-h | S5 | L8 | M15 |
| E2061a-h | S5 | L9 | M15 |
| E2062a-h | S6 | L1 | M15 |
| E2063a-h | S6 | L2 | M15 |
| E2064a-h | S6 | L3 | M15 |
| E2065a-h | S6 | L4 | M15 |
| E2066a-h | S6 | L5 | M15 |
| E2067a-h | S6 | L6 | M15 |
| E2068a-h | S6 | L7 | M15 |
| E2069a-h | S6 | L8 | M15 |
| E2070a-h | S6 | L9 | M15 |
| E2071a-h | S7 | L1 | M15 |
| E2072a-h | S7 | L2 | M15 |
| E2073a-h | S7 | L3 | M15 |
| E2074a-h | S7 | L4 | M15 |
| E2975a-h | S7 | L5 | M15 |
| E2076a-h | S7 | L6 | M15 |
| E2077a-h | S7 | L7 | M15 |
| E2078a-h | S7 | L8 | M15 |
| E2979a-h | S7 | L9 | M15 |
| E2080a-h | S8 | L1 | M15 |
| E2081a-h | S8 | L2 | M15 |
| E2082a-h | S8 | L3 | M15 |
| E2083a-h | S8 | L4 | M15 |
| E2084a-h | S8 | L5 | M15 |
| E2085a-h | S8 | L6 | M15 |
| E2086a-h | S8 | L7 | M15 |
| E2087a-h | S8 | L8 | M15 |
| E2088a-h | S8 | L9 | M15 |
| E2089a-h | S9 | L1 | M15 |
| E2090a-h | S9 | L2 | M15 |
| E2091a-h | S9 | L3 | M15 |
| E2092a-h | S9 | L4 | M15 |
| E2093a-h | S9 | L5 | M15 |
| E2094a-h | S9 | L6 | M15 |
| E2095a-h | S9 | L7 | M15 |
| E2096a-h | S9 | L8 | M15 |
| E2097a-h | S9 | L9 | M15 |
| E2098a-h | S10 | L1 | M15 |
| E2099a-h | S10 | L2 | M15 |
| E2100a-h | S10 | L3 | M15 |
| E2101a-h | S10 | L4 | M15 |
| E2102a-h | S10 | L5 | M15 |
| E2103a-h | S10 | L6 | M15 |
| E2104a-h | S10 | L7 | M15 |
| E2105a-h | S10 | L8 | M15 |
| E2106a-h | S10 | L9 | M15 |
| E2107a-h | S11 | L1 | M15 |
| E2108a-h | S11 | L2 | M15 |
| E2109a-h | S11 | L3 | M15 |
| E2110a-h | S11 | L4 | M15 |
| E2111a-h | S11 | L5 | M15 |
| E2112a-h | S11 | L6 | M15 |
| E2113a-h | S11 | L7 | M15 |
| E2114a-h | S11 | L8 | M15 |
| E2115a-h | S11 | L9 | M15 |
| E2116a-h | S12 | L1 | M15 |
| E2117a-h | S12 | L2 | M15 |
| E2118a-h | S12 | L3 | M15 |
| E2119a-h | S12 | L4 | M15 |
| E2120a-h | S12 | L5 | M15 |
| E2121a-h | S12 | L6 | M15 |
| E2122a-h | S12 | L7 | M15 |
| E2123a-h | S12 | L8 | M15 |
| E2124a-h | S12 | L9 | M15 |
| E2125a-h | S13 | L1 | M15 |
| E2126a-h | S13 | L2 | M15 |
| E2127a-h | S13 | L3 | M15 |
| E2128a-h | S13 | L4 | M15 |
| E2129a-h | S13 | L5 | M15 |
| E2130a-h | S13 | L6 | M15 |
| E2131a-h | S13 | L7 | M15 |
| E2132a-h | S13 | L8 | M15 |
| E2133a-h | S13 | L9 | M15 |
| E2134a-h | S14 | L1 | M15 |
| E2135a-h | S14 | L2 | M15 |
| E2136a-h | S14 | L3 | M15 |
| E2137a-h | S14 | L4 | M15 |
| E2138a-h | S14 | L5 | M15 |
| E2139a-h | S14 | L6 | M15 |
| E2140a-h | S14 | L7 | M15 |
| E2141a-h | S14 | L8 | M15 |
| E2142a-h | S14 | L9 | M15 |
| E2143a-h | S15 | L1 | M15 |
| E2144a-h | S15 | L2 | M15 |
| E2145a-h | S15 | L3 | M15 |
| E2146a-h | S15 | L4 | M15 |
| E2147a-h | S15 | L5 | M15 |
| E2148a-h | S15 | L6 | M15 |
| E2149a-h | S15 | L7 | M15 |
| E2150a-h | S15 | L8 | M15 |
| E2151a-h | S15 | L9 | M15 |
| E2152a-h | S16 | L1 | M15 |
| E2153a-h | S16 | L2 | M15 |
| E2154a-h | S16 | L3 | M15 |
| E2155a-h | S16 | L4 | M15 |
| E2156a-h | S16 | L5 | M15 |
| E2157a-h | S16 | L6 | M15 |
| E2158a-h | S16 | L7 | M15 |
| E2159a-h | S16 | L8 | M15 |
| E2160a-h | S16 | L9 | M15 |
| E2161a-h | S1 | L1 | M16 |
| E2162a-h | S1 | L2 | M16 |
| E2163a-h | S1 | L3 | M16 |
| E2164a-h | S1 | L4 | M16 |
| E2165a-h | S1 | L5 | M16 |
| E2166a-h | S1 | L6 | M16 |
| E2167a-h | S1 | L7 | M16 |
| E2168a-h | S1 | L8 | M16 |
| E2169a-h | S1 | L9 | M16 |
| E2170a-h | S2 | L1 | M16 |
| E2171a-h | S2 | L2 | M16 |
| E2172a-h | S2 | L3 | M16 |
| E2173a-h | S2 | L4 | M16 |
| E2174a-h | S2 | L5 | M16 |
| E2175a-h | S2 | L6 | M16 |
| E2176a-h | S2 | L7 | M16 |
| E2177a-h | S2 | L8 | M16 |
| E2178a-h | S2 | L9 | M16 |
| E2179a-h | S3 | L1 | M16 |
| E2180a-h | S3 | L2 | M16 |
| E2181a-h | S3 | L3 | M16 |
| E2182a-h | S3 | L4 | M16 |
| E2183a-h | S3 | L5 | M16 |
| E2184a-h | S3 | L6 | M16 |
| E2185a-h | S3 | L7 | M16 |
| E2186a-h | S3 | L8 | M16 |
| E2187a-h | S3 | L9 | M16 |
| E2188a-h | S4 | L1 | M16 |
| E2189a-h | S4 | L2 | M16 |
| E2190a-h | S4 | L3 | M16 |
| E2191a-h | S4 | L4 | M16 |
| E2192a-h | S4 | L5 | M16 |
| E2193a-h | S4 | L6 | M16 |
| E2194a-h | S4 | L7 | M16 |
| E2195a-h | S4 | L8 | M16 |
| E2196a-h | S4 | L9 | M16 |
| E2197a-h | S5 | L1 | M16 |
| E2198a-h | S5 | L2 | M16 |
| E2199a-h | S5 | L3 | M16 |
| E2200a-h | S5 | L4 | M16 |
| E2201a-h | S5 | L5 | M16 |
| E2202a-h | S5 | L6 | M16 |
| E2203a-h | S5 | L7 | M16 |
| E2204a-h | S5 | L8 | M16 |
| E2205a-h | S5 | L9 | M16 |
| E2206a-h | S6 | L1 | M16 |
| E2207a-h | S6 | L2 | M16 |
| E2208a-h | S6 | L3 | M16 |
| E2209a-h | S6 | L4 | M16 |
| E2210a-h | S6 | L5 | M16 |
| E2211a-h | S6 | L6 | M16 |
| E2212a-h | S6 | L7 | M16 |
| E2213a-h | S6 | L8 | M16 |
| E2214a-h | S6 | L9 | M16 |
| E2215a-h | S7 | L1 | M16 |
| E2216a-h | S7 | L2 | M16 |
| E2217a-h | S7 | L3 | M16 |
| E2218a-h | S7 | L4 | M16 |
| E2219a-h | S7 | L5 | M16 |
| E2220a-h | S7 | L6 | M16 |
| E2221a-h | S7 | L7 | M16 |
| E2222a-h | S7 | L8 | M16 |
| E2223a-h | S7 | L9 | M16 |
| E2224a-h | S8 | L1 | M16 |
| E2225a-h | S8 | L2 | M16 |
| E2226a-h | S8 | L3 | M16 |
| E2227a-h | S8 | L4 | M16 |
| E2228a-h | S8 | L5 | M16 |
| E2229a-h | S8 | L6 | M16 |
| E2230a-h | S8 | L7 | M16 |
| E2231a-h | S8 | L8 | M16 |
| E2232a-h | S8 | L9 | M16 |
| E2233a-h | S9 | L1 | M16 |
| E2234a-h | S9 | L2 | M16 |
| E2235a-h | S9 | L3 | M16 |
| E2236a-h | S9 | L4 | M16 |
| E2237a-h | S9 | L5 | M16 |
| E2238a-h | S9 | L6 | M16 |
| E2239a-h | S9 | L7 | M16 |
| E2240a-h | S9 | L8 | M16 |
| E2241a-h | S9 | L9 | M16 |
| E2242a-h | S10 | L1 | M16 |
| E2243a-h | S10 | L2 | M16 |
| E2244a-h | S10 | L3 | M16 |
| E2245a-h | S10 | L4 | M16 |
| E2246a-h | S10 | L5 | M16 |
| E2247a-h | S10 | L6 | M16 |
| E2248a-h | S10 | L7 | M16 |
| E2249a-h | S10 | L8 | M16 |
| E2250a-h | S10 | L9 | M16 |
| E2251a-h | S11 | L1 | M16 |
| E2252a-h | S11 | L2 | M16 |
| E2253a-h | S11 | L3 | M16 |
| E2254a-h | S11 | L4 | M16 |
| E2255a-h | S11 | L5 | M16 |
| E2256a-h | S11 | L6 | M16 |
| E2257a-h | S11 | L7 | M16 |
| E2258a-h | S11 | L8 | M16 |
| E2259a-h | S11 | L9 | M16 |
| E2260a-h | S12 | L1 | M16 |
| E2261a-h | S12 | L2 | M16 |
| E2262a-h | S12 | L3 | M16 |
| E2263a-h | S12 | L4 | M16 |
| E2264a-h | S12 | L5 | M16 |
| E2265a-h | S12 | L6 | M16 |
| E2266a-h | S12 | L7 | M16 |
| E2267a-h | S12 | L8 | M16 |
| E2268a-h | S12 | L9 | M16 |
| E2269a-h | S13 | L1 | M16 |
| E2270a-h | S13 | L2 | M16 |
| E2271a-h | S13 | L3 | M16 |
| E2272a-h | S13 | L4 | M16 |
| E2273a-h | S13 | L5 | M16 |
| E2274a-h | S13 | L6 | M16 |
| E2275a-h | S13 | L7 | M16 |
| E2276a-h | S13 | L8 | M16 |
| E2277a-h | S13 | L9 | M16 |
| E2278a-h | S14 | L1 | M16 |
| E2279a-h | S14 | L2 | M16 |
| E2280a-h | S14 | L3 | M16 |
| E2281a-h | S14 | L4 | M16 |
| E2282a-h | S14 | L5 | M16 |
| E2283a-h | S14 | L6 | M16 |
| E2284a-h | S14 | L7 | M16 |
| E2285a-h | S14 | L8 | M16 |
| E2286a-h | S14 | L9 | M16 |
| E2287a-h | S15 | L1 | M16 |
| E2288a-h | S15 | L2 | M16 |
| E2289a-h | S15 | L3 | M16 |
| E2290a-h | S15 | L4 | M16 |
| E2291a-h | S15 | L5 | M16 |
| E2292a-h | S15 | L6 | M16 |
| E2293a-h | S15 | L7 | M16 |
| E2294a-h | S15 | L8 | M16 |
| E2295a-h | S15 | L9 | M16 |
| E2296a-h | S16 | L1 | M16 |
| E2297a-h | S16 | L2 | M16 |
| E2298a-h | S16 | L3 | M16 |
| E2299a-h | S16 | L4 | M16 |
| E2300a-h | S16 | L5 | M16 |
| E2301a-h | S16 | L6 | M16 |
| E2302a-h | S16 | L7 | M16 |
| E2303a-h | S16 | L8 | M16 |
| E2304a-h | S16 | L9 | M16 |
| E2305a-h | S1 | L1 | M17 |
| E2306a-h | S1 | L2 | M17 |
| E2307a-h | S1 | L3 | M17 |
| E2308a-h | S1 | L4 | M17 |
| E2309a-h | S1 | L5 | M17 |
| E2310a-h | S1 | L6 | M17 |
| E2311a-h | S1 | L7 | M17 |
| E2312a-h | S1 | L8 | M17 |
| E2313a-h | S1 | L9 | M17 |
| E2314a-h | S2 | L1 | M17 |
| E2315a-h | S2 | L2 | M17 |
| E2316a-h | S2 | L3 | M17 |
| E2317a-h | S2 | L4 | M17 |
| E2318a-h | S2 | L5 | M17 |
| E2319a-h | S2 | L6 | M17 |
| E2320a-h | S2 | L7 | M17 |
| E2321a-h | S2 | L8 | M17 |
| E2322a-h | S2 | L9 | M17 |
| E2323a-h | S3 | L1 | M17 |
| E2324a-h | S3 | L2 | M17 |
| E2325a-h | S3 | L3 | M17 |
| E2326a-h | S3 | L4 | M17 |
| E2327a-h | S3 | L5 | M17 |
| E2328a-h | S3 | L6 | M17 |
| E2329a-h | S3 | L7 | M17 |
| E2330a-h | S3 | L8 | M17 |
| E2331a-h | S3 | L9 | M17 |
| E2332a-h | S4 | L1 | M17 |
| E2333a-h | S4 | L2 | M17 |
| E2334a-h | S4 | L3 | M17 |
| E2335a-h | S4 | L4 | M17 |
| E2336a-h | S4 | L5 | M17 |
| E2337a-h | S4 | L6 | M17 |
| E2338a-h | S4 | L7 | M17 |
| E2339a-h | S4 | L8 | M17 |
| E2340a-h | S4 | L9 | M17 |
| E2341a-h | S5 | L1 | M17 |
| E2342a-h | S5 | L2 | M17 |
| E2343a-h | S5 | L3 | M17 |
| E2344a-h | S5 | L4 | M17 |
| E2345a-h | S5 | L5 | M17 |
| E2346a-h | S5 | L6 | M17 |
| E2347a-h | S5 | L7 | M17 |
| E2348a-h | S5 | L8 | M17 |
| E2349a-h | S5 | L9 | M17 |
| E2350a-h | S6 | L1 | M17 |
| E2351a-h | S6 | L2 | M17 |
| E2352a-h | S6 | L3 | M17 |
| E2353a-h | S6 | L4 | M17 |
| E2354a-h | S6 | L5 | M17 |
| E2355a-h | S6 | L6 | M17 |
| E2356a-h | S6 | L7 | M17 |
| E2357a-h | S6 | L8 | M17 |
| E2358a-h | S6 | L9 | M17 |
| E2359a-h | S7 | L1 | M17 |
| E2360a-h | S7 | L2 | M17 |
| E2361a-h | S7 | L3 | M17 |
| E2362a-h | S7 | L4 | M17 |
| E2363a-h | S7 | L5 | M17 |
| E2364a-h | S7 | L6 | M17 |
| E2365a-h | S7 | L7 | M17 |
| E2366a-h | S7 | L8 | M17 |
| E2367a-h | S7 | L9 | M17 |
| E2368a-h | S8 | L1 | M17 |
| E2369a-h | S8 | L2 | M17 |
| E2370a-h | S8 | L3 | M17 |
| E2371a-h | S8 | L4 | M17 |
| E2372a-h | S8 | L5 | M17 |
| E2373a-h | S8 | L6 | M17 |
| E2374a-h | S8 | L7 | M17 |
| E2375a-h | S8 | L8 | M17 |
| E2376a-h | S8 | L9 | M17 |
| E2377a-h | S9 | L1 | M17 |
| E2378a-h | S9 | L2 | M17 |
| E2379a-h | S9 | L3 | M17 |
| E2380a-h | S9 | L4 | M17 |
| E2381a-h | S9 | L5 | M17 |
| E2382a-h | S9 | L6 | M17 |
| E2383a-h | S9 | L7 | M17 |
| E2384a-h | S9 | L8 | M17 |
| E2385a-h | S9 | L9 | M17 |
| E2386a-h | S10 | L1 | M17 |
| E2387a-h | S10 | L2 | M17 |
| E2388a-h | S10 | L3 | M17 |
| E2389a-h | S10 | L4 | M17 |
| E2390a-h | S10 | L5 | M17 |
| E2391a-h | S10 | L6 | M17 |
| E2392a-h | S10 | L7 | M17 |
| E2393a-h | S10 | L8 | M17 |
| E2394a-h | S10 | L9 | M17 |
| E2395a-h | S11 | L1 | M17 |
| E2396a-h | S11 | L2 | M17 |
| E2397a-h | S11 | L3 | M17 |
| E2398a-h | S11 | L4 | M17 |
| E2399a-h | S11 | L5 | M17 |
| E2400a-h | S11 | L6 | M17 |
| E2401a-h | S11 | L7 | M17 |
| E2402a-h | S11 | L8 | M17 |
| E2403a-h | S11 | L9 | M17 |
| E2404a-h | S12 | L1 | M17 |
| E2405a-h | S12 | L2 | M17 |
| E2406a-h | S12 | L3 | M17 |
| E2407a-h | S12 | L4 | M17 |
| E2408a-h | S12 | L5 | M17 |
| E2409a-h | S12 | L6 | M17 |
| E2410a-h | S12 | L7 | M17 |
| E2411a-h | S12 | L8 | M17 |
| E2412a-h | S12 | L9 | M17 |
| E2413a-h | S13 | L1 | M17 |
| E2414a-h | S13 | L2 | M17 |
| E2415a-h | S13 | L3 | M17 |
| E2416a-h | S13 | L4 | M17 |
| E2417a-h | S13 | L5 | M17 |
| E2418a-h | S13 | L6 | M17 |
| E2419a-h | S13 | L7 | M17 |
| E2420a-h | S13 | L8 | M17 |
| E2421a-h | S13 | L9 | M17 |
| E2422a-h | S14 | L1 | M17 |
| E2423a-h | S14 | L2 | M17 |
| E2424a-h | S14 | L3 | M17 |
| E2425a-h | S14 | L4 | M17 |
| E2426a-h | S14 | L5 | M17 |
| E2427a-h | S14 | L6 | M17 |
| E2428a-h | S14 | L7 | M17 |
| E2429a-h | S14 | L8 | M17 |
| E2430a-h | S14 | L9 | M17 |
| E2431a-h | S15 | L1 | M17 |
| E2432a-h | S15 | L2 | M17 |
| E2433a-h | S15 | L3 | M17 |
| E2434a-h | S15 | L4 | M17 |
| E2435a-h | S15 | L5 | M17 |
| E2436a-h | S15 | L6 | M17 |
| E2437a-h | S15 | L7 | M17 |
| E2438a-h | S15 | L8 | M17 |
| E2439a-h | S15 | L9 | M17 |
| E2440a-h | S16 | L1 | M17 |
| E2441a-h | S16 | L2 | M17 |
| E2442a-h | S16 | L3 | M17 |
| E2443a-h | S16 | L4 | M17 |
| E2444a-h | S16 | L5 | M17 |
| E2445a-h | S16 | L6 | M17 |
| E2446a-h | S16 | L7 | M17 |
| E2447a-h | S16 | L8 | M17 |
| E2448a-h | S16 | L9 | M17 |
| E2449a-h | S1 | L1 | M18 |
| E2450a-h | S1 | L2 | M18 |
| E2451a-h | S1 | L3 | M18 |
| E2452a-h | S1 | L4 | M18 |
| E2453a-h | S1 | L5 | M18 |
| E2454a-h | S1 | L6 | M18 |
| E2455a-h | S1 | L7 | M18 |
| E2456a-h | S1 | L8 | M18 |
| E2457a-h | S1 | L9 | M18 |
| E2458a-h | S2 | L1 | M18 |
| E2459a-h | S2 | L2 | M18 |
| E2460a-h | S2 | L3 | M18 |
| E2461a-h | S2 | L4 | M18 |
| E2462a-h | S2 | L5 | M18 |
| E2463a-h | S2 | L6 | M18 |
| E2464a-h | S2 | L7 | M18 |
| E2465a-h | S2 | L8 | M18 |
| E2466a-h | S2 | L9 | M18 |
| E2467a-h | S3 | L1 | M18 |
| E2468a-h | S3 | L2 | M18 |
| E2469a-h | S3 | L3 | M18 |
| E2470a-h | S3 | L4 | M18 |
| E2471a-h | S3 | L5 | M18 |
| E2472a-h | S3 | L6 | M18 |
| E2473a-h | S3 | L7 | M18 |
| E2474a-h | S3 | L8 | M18 |
| E2475a-h | S3 | L9 | M18 |
| E2476a-h | S4 | L1 | M18 |
| E2477a-h | S4 | L2 | M18 |
| E2478a-h | S4 | L3 | M18 |
| E2479a-h | S4 | L4 | M18 |
| E2480a-h | S4 | L5 | M18 |
| E2481a-h | S4 | L6 | M18 |
| E2482a-h | S4 | L7 | M18 |
| E2483a-h | S4 | L8 | M18 |
| E2484a-h | S4 | L9 | M18 |
| E2485a-h | S5 | L1 | M18 |
| E2486a-h | S5 | L2 | M18 |
| E2487a-h | S5 | L3 | M18 |
| E2488a-h | S5 | L4 | M18 |
| E2489a-h | S5 | L5 | M18 |
| E2490a-h | S5 | L6 | M18 |
| E2491a-h | S5 | L7 | M18 |
| E2492a-h | S5 | L8 | M18 |
| E2493a-h | S5 | L9 | M18 |
| E2494a-h | S6 | L1 | M18 |
| E2495a-h | S6 | L2 | M18 |
| E2496a-h | S6 | L3 | M18 |
| E2497a-h | S6 | L4 | M18 |
| E2498a-h | S6 | L5 | M18 |
| E2499a-h | S6 | L6 | M18 |
| E2500a-h | S6 | L7 | M18 |
| E2501a-h | S6 | L8 | M18 |
| E2502a-h | S6 | L9 | M18 |
| E2503a-h | S7 | L1 | M18 |
| E2504a-h | S7 | L2 | M18 |
| E2505a-h | S7 | L3 | M18 |
| E2506a-h | S7 | L4 | M18 |
| E2507a-h | S7 | L5 | M18 |
| E2508a-h | S7 | L6 | M18 |
| E2509a-h | S7 | L7 | M18 |
| E2510a-h | S7 | L8 | M18 |
| E2511a-h | S7 | L9 | M18 |
| E2512a-h | S8 | L1 | M18 |
| E2513a-h | S8 | L2 | M18 |
| E2514a-h | S8 | L3 | M18 |
| E2515a-h | S8 | L4 | M18 |
| E2516a-h | S8 | L5 | M18 |
| E2517a-h | S8 | L6 | M18 |
| E2518a-h | S8 | L7 | M18 |
| E2519a-h | S8 | L8 | M18 |
| E2520a-h | S8 | L9 | M18 |
| E2521a-h | S9 | L1 | M18 |
| E2522a-h | S9 | L2 | M18 |
| E2523a-h | S9 | L3 | M18 |
| E2524a-h | S9 | L4 | M18 |
| E2525a-h | S9 | L5 | M18 |
| E2526a-h | S9 | L6 | M18 |
| E2527a-h | S9 | L7 | M18 |
| E2528a-h | S9 | L8 | M18 |
| E2529a-h | S9 | L9 | M18 |
| E2530a-h | S10 | L1 | M18 |
| E2531a-h | S10 | L2 | M18 |
| E2532a-h | S10 | L3 | M18 |
| E2533a-h | S10 | L4 | M18 |
| E2534a-h | S10 | L5 | M18 |
| E2535a-h | S10 | L6 | M18 |
| E2536a-h | S10 | L7 | M18 |
| E2537a-h | S10 | L8 | M18 |
| E2538a-h | S10 | L9 | M18 |
| E2539a-h | S11 | L1 | M18 |
| E2540a-h | S11 | L2 | M18 |
| E2541a-h | S11 | L3 | M18 |
| E2542a-h | S11 | L4 | M18 |
| E2543a-h | S11 | L5 | M18 |
| E2544a-h | S11 | L6 | M18 |
| E2545a-h | S11 | L7 | M18 |
| E2546a-h | S11 | L8 | M18 |
| E2547a-h | S11 | L9 | M18 |
| E2548a-h | S12 | L1 | M18 |
| E2549a-h | S12 | L2 | M18 |
| E2550a-h | S12 | L3 | M18 |
| E2551a-h | S12 | L4 | M18 |
| E2552a-h | S12 | L5 | M18 |
| E2553a-h | S12 | L6 | M18 |
| E2554a-h | S12 | L7 | M18 |
| E2555a-h | S12 | L8 | M18 |
| E2556a-h | S12 | L9 | M18 |
| E2557a-h | S13 | L1 | M18 |
| E2558a-h | S13 | L2 | M18 |
| E2559a-h | S13 | L3 | M18 |
| E2560a-h | S13 | L4 | M18 |
| E2561a-h | S13 | L5 | M18 |
| E2562a-h | S13 | L6 | M18 |
| E2563a-h | S13 | L7 | M18 |
| E2564a-h | S13 | L8 | M18 |
| E2565a-h | S13 | L9 | M18 |
| E2566a-h | S14 | L1 | M18 |
| E2567a-h | S14 | L2 | M18 |
| E2568a-h | S14 | L3 | M18 |
| E2569a-h | S14 | L4 | M18 |
| E2570a-h | S14 | L5 | M18 |
| E2571a-h | S14 | L6 | M18 |
| E2572a-h | S14 | L7 | M18 |
| E2573a-h | S14 | L8 | M18 |
| E2574a-h | S14 | L9 | M18 |
| E2575a-h | S15 | L1 | M18 |
| E2576a-h | S15 | L2 | M18 |
| E2577a-h | S15 | L3 | M18 |
| E2578a-h | S15 | L4 | M18 |
| E2579a-h | S15 | L5 | M18 |
| E2580a-h | S15 | L6 | M18 |
| E2581a-h | S15 | L7 | M18 |
| E2582a-h | S15 | L8 | M18 |
| E2583a-h | S15 | L9 | M18 |
| E2584a-h | S16 | L1 | M18 |
| E2585a-h | S16 | L2 | M18 |
| E2586a-h | S16 | L3 | M18 |
| E2587a-h | S16 | L4 | M18 |
| E2588a-h | S16 | L5 | M18 |
| E2589a-h | S16 | L6 | M18 |
| E2590a-h | S16 | L7 | M18 |
| E2591a-h | S16 | L8 | M18 |
| E2592a-h | S16 | L9 | M18 |
| E2593a-h | S1 | L1 | M19 |
| E2594a-h | S1 | L2 | M19 |
| E2595a-h | S1 | L3 | M19 |
| E2596a-h | S1 | L4 | M19 |
| E2597a-h | S1 | L5 | M19 |
| E2598a-h | S1 | L6 | M19 |
| E2599a-h | S1 | L7 | M19 |
| E2600a-h | S1 | L8 | M19 |
| E2601a-h | S1 | L9 | M19 |
| E2602a-h | S2 | L1 | M19 |
| E2603a-h | S2 | L2 | M19 |
| E2604a-h | S2 | L3 | M19 |
| E2605a-h | S2 | L4 | M19 |
| E2606a-h | S2 | L5 | M19 |
| E2607a-h | S2 | L6 | M19 |
| E2608a-h | S2 | L7 | M19 |
| E2609a-h | S2 | L8 | M19 |
| E2610a-h | S2 | L9 | M19 |
| E2611a-h | S3 | L1 | M19 |
| E2612a-h | S3 | L2 | M19 |
| E2613a-h | S3 | L3 | M19 |
| E2614a-h | S3 | L4 | M19 |
| E2615a-h | S3 | L5 | M19 |
| E2616a-h | S3 | L6 | M19 |
| E2617a-h | S3 | L7 | M19 |
| E2618a-h | S3 | L8 | M19 |
| E2619a-h | S3 | L9 | M19 |
| E2620a-h | S4 | L1 | M19 |
| E2621a-h | S4 | L2 | M19 |
| E2622a-h | S4 | L3 | M19 |
| E2623a-h | S4 | L4 | M19 |
| E2624a-h | S4 | L5 | M19 |
| E2625a-h | S4 | L6 | M19 |
| E2626a-h | S4 | L7 | M19 |
| E2627a-h | S4 | L8 | M19 |
| E2628a-h | S4 | L9 | M19 |
| E2629a-h | S5 | L1 | M19 |
| E2630a-h | S5 | L2 | M19 |
| E2631a-h | S5 | L3 | M19 |
| E2632a-h | S5 | L4 | M19 |
| E2633a-h | S5 | L5 | M19 |
| E2634a-h | S5 | L6 | M19 |
| E2635a-h | S5 | L7 | M19 |
| E2636a-h | S5 | L8 | M19 |
| E2637a-h | S5 | L9 | M19 |
| E2638a-h | S6 | L1 | M19 |
| E2639a-h | S6 | L2 | M19 |
| E2640a-h | S6 | L3 | M19 |
| E2641a-h | S6 | L4 | M19 |
| E2642a-h | S6 | L5 | M19 |
| E2643a-h | S6 | L6 | M19 |
| E2644a-h | S6 | L7 | M19 |
| E2645a-h | S6 | L8 | M19 |
| E2646a-h | S6 | L9 | M19 |
| E2647a-h | S7 | L1 | M19 |
| E2648a-h | S7 | L2 | M19 |
| E2649a-h | S7 | L3 | M19 |
| E2650a-h | S7 | L4 | M19 |
| E2651a-h | S7 | L5 | M19 |
| E2652a-h | S7 | L6 | M19 |
| E2653a-h | S7 | L7 | M19 |
| E2654a-h | S7 | L8 | M19 |
| E2655a-h | S7 | L9 | M19 |
| E2656a-h | S8 | L1 | M19 |
| E2657a-h | S8 | L2 | M19 |
| E2658a-h | S8 | L3 | M19 |
| E2659a-h | S8 | L4 | M19 |
| E2660a-h | S8 | L5 | M19 |
| E2661a-h | S8 | L6 | M19 |
| E2662a-h | S8 | L7 | M19 |
| E2663a-h | S8 | L8 | M19 |
| E2664a-h | S8 | L9 | M19 |
| E2665a-h | S9 | L1 | M19 |
| E2666a-h | S9 | L2 | M19 |
| E2667a-h | S9 | L3 | M19 |
| E2668a-h | S9 | L4 | M19 |
| E2669a-h | S9 | L5 | M19 |
| E2670a-h | S9 | L6 | M19 |
| E2671a-h | S9 | L7 | M19 |
| E2672a-h | S9 | L8 | M19 |
| E2673a-h | S9 | L9 | M19 |
| E2674a-h | S10 | L1 | M19 |
| E2675a-h | S10 | L2 | M19 |
| E2676a-h | S10 | L3 | M19 |
| E2677a-h | S10 | L4 | M19 |
| E2678a-h | S10 | L5 | M19 |
| E2679a-h | S10 | L6 | M19 |
| E2680a-h | S10 | L7 | M19 |
| E2681a-h | S10 | L8 | M19 |
| E2682a-h | S10 | L9 | M19 |
| E2683a-h | S11 | L1 | M19 |
| E2684a-h | S11 | L2 | M19 |
| E2685a-h | S11 | L3 | M19 |
| E2686a-h | S11 | L4 | M19 |
| E2687a-h | S11 | L5 | M19 |
| E2688a-h | S11 | L6 | M19 |
| E2689a-h | S11 | L7 | M19 |
| E2690a-h | S11 | L8 | M19 |
| E2691a-h | S11 | L9 | M19 |
| E2692a-h | S12 | L1 | M19 |
| E2693a-h | S12 | L2 | M19 |
| E2694a-h | S12 | L3 | M19 |
| E2695a-h | S12 | L4 | M19 |
| E2696a-h | S12 | L5 | M19 |
| E2697a-h | S12 | L6 | M19 |
| E2698a-h | S12 | L7 | M19 |
| E2699a-h | S12 | L8 | M19 |
| E2700a-h | S12 | L9 | M19 |
| E2701a-h | S13 | L1 | M19 |
| E2702a-h | S13 | L2 | M19 |
| E2703a-h | S13 | L3 | M19 |
| E2704a-h | S13 | L4 | M19 |
| E2705a-h | S13 | L5 | M19 |
| E2706a-h | S13 | L6 | M19 |
| E2707a-h | S13 | L7 | M19 |
| E2708a-h | S13 | L8 | M19 |
| E2709a-h | S13 | L9 | M19 |
| E2710a-h | S14 | L1 | M19 |
| E2711a-h | S14 | L2 | M19 |
| E2712a-h | S14 | L3 | M19 |
| E2713a-h | S14 | L4 | M19 |
| E2714a-h | S14 | L5 | M19 |
| E2715a-h | S14 | L6 | M19 |
| E2716a-h | S14 | L7 | M19 |
| E2717a-h | S14 | L8 | M19 |
| E2718a-h | S14 | L9 | M19 |
| E2719a-h | S15 | L1 | M19 |
| E2720a-h | S15 | L2 | M19 |
| E2721a-h | S15 | L3 | M19 |
| E2722a-h | S15 | L4 | M19 |
| E2723a-h | S15 | L5 | M19 |
| E2724a-h | S15 | L6 | M19 |
| E2725a-h | S15 | L7 | M19 |
| E2726a-h | S15 | L8 | M19 |
| E2727a-h | S15 | L9 | M19 |
| E2728a-h | S16 | L1 | M19 |
| E2729a-h | S16 | L2 | M19 |
| E2730a-h | S16 | L3 | M19 |
| E2731a-h | S16 | L4 | M19 |
| E2732a-h | S16 | L5 | M19 |
| E2733a-h | S16 | L6 | M19 |
| E2734a-h | S16 | L7 | M19 |
| E2735a-h | S16 | L8 | M19 |
| E2736a-h | S16 | L9 | M19 |
| E2737a-h | S1 | L1 | M20 |
| E2738a-h | S1 | L2 | M20 |
| E2739a-h | S1 | L3 | M20 |
| E2740a-h | S1 | L4 | M20 |
| E2741a-h | S1 | L5 | M20 |
| E2742a-h | S1 | L6 | M20 |
| E2743a-h | S1 | L7 | M20 |
| E2744a-h | S1 | L8 | M20 |
| E2745a-h | S1 | L9 | M20 |
| E2746a-h | S2 | L1 | M20 |
| E2747a-h | S2 | L2 | M20 |
| E2748a-h | S2 | L3 | M20 |
| E2749a-h | S2 | L4 | M20 |
| E2750a-h | S2 | L5 | M20 |
| E2751a-h | S2 | L6 | M20 |
| E2752a-h | S2 | L7 | M20 |
| E2753a-h | S2 | L8 | M20 |
| E2754a-h | S2 | L9 | M20 |
| E2755a-h | S3 | L1 | M20 |
| E2756a-h | S3 | L2 | M20 |
| E2757a-h | S3 | L3 | M20 |
| E2758a-h | S3 | L4 | M20 |
| E2759a-h | S3 | L5 | M20 |
| E2760a-h | S3 | L6 | M20 |
| E2761a-h | S3 | L7 | M20 |
| E2762a-h | S3 | L8 | M20 |
| E2763a-h | S3 | L9 | M20 |
| E2764a-h | S4 | L1 | M20 |
| E2765a-h | S4 | L2 | M20 |
| E2766a-h | S4 | L3 | M20 |
| E2767a-h | S4 | L4 | M20 |
| E2768a-h | S4 | L5 | M20 |
| E2769a-h | S4 | L6 | M20 |
| E2770a-h | S4 | L7 | M20 |
| E2771a-h | S4 | L8 | M20 |
| E2772a-h | S4 | L9 | M20 |
| E2773a-h | S5 | L1 | M20 |
| E2774a-h | S5 | L2 | M20 |
| E2775a-h | S5 | L3 | M20 |
| E2776a-h | S5 | L4 | M20 |
| E2777a-h | S5 | L5 | M20 |
| E2778a-h | S5 | L6 | M20 |
| E2779a-h | S5 | L7 | M20 |
| E2780a-h | S5 | L8 | M20 |
| E2781a-h | S5 | L9 | M20 |
| E2782a-h | S6 | L1 | M20 |
| E2783a-h | S6 | L2 | M20 |
| E2784a-h | S6 | L3 | M20 |
| E2785a-h | S6 | L4 | M20 |
| E2786a-h | S6 | L5 | M20 |
| E2787a-h | S6 | L6 | M20 |
| E2788a-h | S6 | L7 | M20 |
| E2789a-h | S6 | L8 | M20 |
| E2790a-h | S6 | L9 | M20 |
| E2791a-h | S7 | L1 | M20 |
| E2792a-h | S7 | L2 | M20 |
| E2793a-h | S7 | L3 | M20 |
| E2794a-h | S7 | L4 | M20 |
| E2795a-h | S7 | L5 | M20 |
| E2796a-h | S7 | L6 | M20 |
| E2797a-h | S7 | L7 | M20 |
| E2798a-h | S7 | L8 | M20 |
| E2799a-h | S7 | L9 | M20 |
| E2800a-h | S8 | L1 | M20 |
| E2801a-h | S8 | L2 | M20 |
| E2802a-h | S8 | L3 | M20 |
| E2803a-h | S8 | L4 | M20 |
| E2804a-h | S8 | L5 | M20 |
| E2805a-h | S8 | L6 | M20 |
| E2806a-h | S8 | L7 | M20 |
| E2807a-h | S8 | L8 | M20 |
| E2808a-h | S8 | L9 | M20 |
| E2809a-h | S9 | L1 | M20 |
| E2810a-h | S9 | L2 | M20 |
| E2811a-h | S9 | L3 | M20 |
| E2812a-h | S9 | L4 | M20 |
| E2813a-h | S9 | L5 | M20 |
| E2814a-h | S9 | L6 | M20 |
| E2815a-h | S9 | L7 | M20 |
| E2816a-h | S9 | L8 | M20 |
| E2817a-h | S9 | L9 | M20 |
| E2818a-h | S10 | L1 | M20 |
| E2819a-h | S10 | L2 | M20 |
| E2820a-h | S10 | L3 | M20 |
| E2821a-h | S10 | L4 | M20 |
| E2822a-h | S10 | L5 | M20 |
| E2823a-h | S10 | L6 | M20 |
| E2824a-h | S10 | L7 | M20 |
| E2825a-h | S10 | L8 | M20 |
| E2826a-h | S10 | L9 | M20 |
| E2827a-h | S11 | L1 | M20 |
| E2828a-h | S11 | L2 | M20 |
| E2829a-h | S11 | L3 | M20 |
| E2830a-h | S11 | L4 | M20 |
| E2831a-h | S11 | L5 | M20 |
| E2832a-h | S11 | L6 | M20 |
| E2833a-h | S11 | L7 | M20 |
| E2834a-h | S11 | L8 | M20 |
| E2835a-h | S11 | L9 | M20 |
| E2836a-h | S12 | L1 | M20 |
| E2837a-h | S12 | L2 | M20 |
| E2838a-h | S12 | L3 | M20 |
| E2839a-h | S12 | L4 | M20 |
| E2840a-h | S12 | L5 | M20 |
| E2841a-h | S12 | L6 | M20 |
| E2842a-h | S12 | L7 | M20 |
| E2843a-h | S12 | L8 | M20 |
| E2844a-h | S12 | L9 | M20 |
| E2845a-h | S13 | L1 | M20 |
| E2846a-h | S13 | L2 | M20 |
| E2847a-h | S13 | L3 | M20 |
| E2848a-h | S13 | L4 | M20 |
| E2849a-h | S13 | L5 | M20 |
| E2850a-h | S13 | L6 | M20 |
| E2851a-h | S13 | L7 | M20 |
| E2852a-h | S13 | L8 | M20 |
| E2853a-h | S13 | L9 | M20 |
| E2854a-h | S14 | L1 | M20 |
| E2855a-h | S14 | L2 | M20 |
| E2856a-h | S14 | L3 | M20 |
| E2857a-h | S14 | L4 | M20 |
| E2858a-h | S14 | L5 | M20 |
| E2859a-h | S14 | L6 | M20 |
| E2860a-h | S14 | L7 | M20 |
| E2861a-h | S14 | L8 | M20 |
| E2862a-h | S14 | L9 | M20 |
| E2863a-h | S15 | L1 | M20 |
| E2864a-h | S15 | L2 | M20 |
| E2865a-h | S15 | L3 | M20 |
| E2866a-h | S15 | L4 | M20 |
| E2867a-h | S15 | L5 | M20 |
| E2868a-h | S15 | L6 | M20 |
| E2869a-h | S15 | L7 | M20 |
| E2870a-h | S15 | L8 | M20 |
| E2871a-h | S15 | L9 | M20 |
| E2872a-h | S16 | L1 | M20 |
| E2873a-h | S16 | L2 | M20 |
| E2874a-h | S16 | L3 | M20 |
| E2875a-h | S16 | L4 | M20 |
| E2876a-h | S16 | L5 | M20 |
| E2877a-h | S16 | L6 | M20 |
| E2878a-h | S16 | L7 | M20 |
| E2879a-h | S16 | L8 | M20 |
| E2880a-h | S16 | L9 | M20 |
| E2881a-h | S1 | L1 | M21 |
| E2882a-h | S1 | L2 | M21 |
| E2883a-h | S1 | L3 | M21 |
| E2884a-h | S1 | L4 | M21 |
| E2885a-h | S1 | L5 | M21 |
| E2886a-h | S1 | L6 | M21 |
| E2887a-h | S1 | L7 | M21 |
| E2888a-h | S1 | L8 | M21 |
| E2889a-h | S1 | L9 | M21 |
| E2890a-h | S2 | L1 | M21 |
| E2891a-h | S2 | L2 | M21 |
| E2892a-h | S2 | L3 | M21 |
| E2893a-h | S2 | L4 | M21 |
| E2894a-h | S2 | L5 | M21 |
| E2895a-h | S2 | L6 | M21 |
| E2896a-h | S2 | L7 | M21 |
| E2897a-h | S2 | L8 | M21 |
| E2898a-h | S2 | L9 | M21 |
| E2899a-h | S3 | L1 | M21 |
| E2900a-h | S3 | L2 | M21 |
| E2901a-h | S3 | L3 | M21 |
| E2902a-h | S3 | L4 | M21 |
| E2903a-h | S3 | L5 | M21 |
| E2904a-h | S3 | L6 | M21 |
| E2905a-h | S3 | L7 | M21 |
| E2906a-h | S3 | L8 | M21 |
| E2907a-h | S3 | L9 | M21 |
| E2908a-h | S4 | L1 | M21 |
| E2909a-h | S4 | L2 | M21 |
| E2910a-h | S4 | L3 | M21 |
| E2911a-h | S4 | L4 | M21 |
| E2912a-h | S4 | L5 | M21 |
| E2913a-h | S4 | L6 | M21 |
| E2914a-h | S4 | L7 | M21 |
| E2915a-h | S4 | L8 | M21 |
| E2916a-h | S4 | L9 | M21 |
| E2917a-h | S5 | L1 | M21 |
| E2918a-h | S5 | L2 | M21 |
| E2919a-h | S5 | L3 | M21 |
| E2920a-h | S5 | L4 | M21 |
| E2921a-h | S5 | L5 | M21 |
| E2922a-h | S5 | L6 | M21 |
| E2923a-h | S5 | L7 | M21 |
| E2924a-h | S5 | L8 | M21 |
| E2925a-h | S5 | L9 | M21 |
| E2926a-h | S6 | L1 | M21 |
| E2927a-h | S6 | L2 | M21 |
| E2928a-h | S6 | L3 | M21 |
| E2929a-h | S6 | L4 | M21 |
| E2930a-h | S6 | L5 | M21 |
| E2931a-h | S6 | L6 | M21 |
| E2932a-h | S6 | L7 | M21 |
| E2933a-h | S6 | L8 | M21 |
| E2934a-h | S6 | L9 | M21 |
| E2935a-h | S7 | L1 | M21 |
| E2936a-h | S7 | L2 | M21 |
| E2937a-h | S7 | L3 | M21 |
| E2938a-h | S7 | L4 | M21 |
| E2939a-h | S7 | L5 | M21 |
| E2940a-h | S7 | L6 | M21 |
| E2941a-h | S7 | L7 | M21 |
| E2942a-h | S7 | L8 | M21 |
| E2943a-h | S7 | L9 | M21 |
| E2944a-h | S8 | L1 | M21 |
| E2945a-h | S8 | L2 | M21 |
| E2946a-h | S8 | L3 | M21 |
| E2947a-h | S8 | L4 | M21 |
| E2948a-h | S8 | L5 | M21 |
| E2949a-h | S8 | L6 | M21 |
| E2950a-h | S8 | L7 | M21 |
| E2951a-h | S8 | L8 | M21 |
| E2952a-h | S8 | L9 | M21 |
| E2953a-h | S9 | L1 | M21 |
| E2954a-h | S9 | L2 | M21 |
| E2955a-h | S9 | L3 | M21 |
| E2956a-h | S9 | L4 | M21 |
| E2957a-h | S9 | L5 | M21 |
| E2958a-h | S9 | L6 | M21 |
| E2959a-h | S9 | L7 | M21 |
| E2960a-h | S9 | L8 | M21 |
| E2961a-h | S9 | L9 | M21 |
| E2962a-h | S10 | L1 | M21 |
| E2963a-h | S10 | L2 | M21 |
| E2964a-h | S10 | L3 | M21 |
| E2965a-h | S10 | L4 | M21 |
| E2966a-h | S10 | L5 | M21 |
| E2967a-h | S10 | L6 | M21 |
| E2968a-h | S10 | L7 | M21 |
| E2969a-h | S10 | L8 | M21 |
| E2970a-h | S10 | L9 | M21 |
| E2971a-h | S11 | L1 | M21 |
| E2972a-h | S11 | L2 | M21 |
| E2973a-h | S11 | L3 | M21 |
| E2974a-h | S11 | L4 | M21 |
| E2975a-h | S11 | L5 | M21 |
| E2976a-h | S11 | L6 | M21 |
| E2977a-h | S11 | L7 | M21 |
| E2978a-h | S11 | L8 | M21 |
| E2979a-h | S11 | L9 | M21 |
| E2980a-h | S12 | L1 | M21 |
| E2981a-h | S12 | L2 | M21 |
| E2982a-h | S12 | L3 | M21 |
| E2983a-h | S12 | L4 | M21 |
| E2984a-h | S12 | L5 | M21 |
| E2985a-h | S12 | L6 | M21 |
| E2986a-h | S12 | L7 | M21 |
| E2987a-h | S12 | L8 | M21 |
| E2988a-h | S12 | L9 | M21 |
| E2989a-h | S13 | L1 | M21 |
| E2990a-h | S13 | L2 | M21 |
| E2991a-h | S13 | L3 | M21 |
| E2992a-h | S13 | L4 | M21 |
| E2993a-h | S13 | L5 | M21 |
| E2994a-h | S13 | L6 | M21 |
| E2995a-h | S13 | L7 | M21 |
| E2996a-h | S13 | L8 | M21 |
| E2997a-h | S13 | L9 | M21 |
| E2998a-h | S14 | L1 | M21 |
| E2999a-h | S14 | L2 | M21 |
| E3000a-h | S14 | L3 | M21 |
| E3001a-h | S14 | L4 | M21 |
| E3002a-h | S14 | L5 | M21 |
| E3003a-h | S14 | L6 | M21 |
| E3004a-h | S14 | L7 | M21 |
| E3005a-h | S14 | L8 | M21 |
| E3006a-h | S14 | L9 | M21 |
| E3007a-h | S15 | L1 | M21 |
| E3008a-h | S15 | L2 | M21 |
| E3009a-h | S15 | L3 | M21 |
| E3010a-h | S15 | L4 | M21 |
| E3011a-h | S15 | L5 | M21 |
| E3012a-h | S15 | L6 | M21 |
| E3013a-h | S15 | L7 | M21 |
| E3014a-h | S15 | L8 | M21 |
| E3015a-h | S15 | L9 | M21 |
| E3016a-h | S16 | L1 | M21 |
| E3017a-h | S16 | L2 | M21 |
| E3018a-h | S16 | L3 | M21 |
| E3019a-h | S16 | L4 | M21 |
| E3020a-h | S16 | L5 | M21 |
| E3021a-h | S16 | L6 | M21 |
| E3022a-h | S16 | L7 | M21 |
| E3023a-h | S16 | L8 | M21 |
| E3024a-h | S16 | L9 | M21 |
| E3025a-h | S1 | L1 | M22 |
| E3026a-h | S1 | L2 | M22 |
| E3027a-h | S1 | L3 | M22 |
| E3028a-h | S1 | L4 | M22 |
| E3029a-h | S1 | L5 | M22 |
| E3030a-h | S1 | L6 | M22 |
| E3031a-h | S1 | L7 | M22 |
| E3032a-h | S1 | L8 | M22 |
| E3033a-h | S1 | L9 | M22 |
| E3034a-h | S2 | L1 | M22 |
| E3035a-h | S2 | L2 | M22 |
| E3036a-h | S2 | L3 | M22 |
| E3037a-h | S2 | L4 | M22 |
| E3038a-h | S2 | L5 | M22 |
| E3039a-h | S2 | L6 | M22 |
| E3040a-h | S2 | L7 | M22 |
| E3041a-h | S2 | L8 | M22 |
| E3042a-h | S2 | L9 | M22 |
| E3043a-h | S3 | L1 | M22 |
| E3044a-h | S3 | L2 | M22 |
| E3045a-h | S3 | L3 | M22 |
| E3046a-h | S3 | L4 | M22 |
| E3047a-h | S3 | L5 | M22 |
| E3048a-h | S3 | L6 | M22 |
| E3049a-h | S3 | L7 | M22 |
| E3050a-h | S3 | L8 | M22 |
| E3051a-h | S3 | L9 | M22 |
| E3052a-h | S4 | L1 | M22 |
| E3053a-h | S4 | L2 | M22 |
| E3054a-h | S4 | L3 | M22 |
| E3055a-h | S4 | L4 | M22 |
| E3056a-h | S4 | L5 | M22 |
| E3057a-h | S4 | L6 | M22 |
| E3058a-h | S4 | L7 | M22 |
| E3059a-h | S4 | L8 | M22 |
| E3060a-h | S4 | L9 | M22 |
| E3061a-h | S5 | L1 | M22 |
| E3062a-h | S5 | L2 | M22 |
| E3063a-h | S5 | L3 | M22 |
| E3064a-h | S5 | L4 | M22 |
| E3065a-h | S5 | L5 | M22 |
| E3066a-h | S5 | L6 | M22 |
| E3067a-h | S5 | L7 | M22 |
| E3068a-h | S5 | L8 | M22 |
| E3069a-h | S5 | L9 | M22 |
| E3070a-h | S6 | L1 | M22 |
| E3071a-h | S6 | L2 | M22 |
| E3072a-h | S6 | L3 | M22 |
| E3073a-h | S6 | L4 | M22 |
| E3074a-h | S6 | L5 | M22 |
| E3075a-h | S6 | L6 | M22 |
| E3076a-h | S6 | L7 | M22 |
| E3077a-h | S6 | L8 | M22 |
| E3078a-h | S6 | L9 | M22 |
| E3079a-h | S7 | L1 | M22 |
| E3080a-h | S7 | L2 | M22 |
| E3081a-h | S7 | L3 | M22 |
| E3082a-h | S7 | L4 | M22 |
| E3083a-h | S7 | L5 | M22 |
| E3084a-h | S7 | L6 | M22 |
| E3085a-h | S7 | L7 | M22 |
| E3086a-h | S7 | L8 | M22 |
| E3087a-h | S7 | L9 | M22 |
| E3088a-h | S8 | L1 | M22 |
| E3089a-h | S8 | L2 | M22 |
| E3090a-h | S8 | L3 | M22 |
| E3091a-h | S8 | L4 | M22 |
| E3092a-h | S8 | L5 | M22 |
| E3093a-h | S8 | L6 | M22 |
| E3094a-h | S8 | L7 | M22 |
| E3095a-h | S8 | L8 | M22 |
| E3096a-h | S8 | L9 | M22 |
| E3097a-h | S9 | L1 | M22 |
| E3098a-h | S9 | L2 | M22 |
| E3099a-h | S9 | L3 | M22 |
| E3100a-h | S9 | L4 | M22 |
| E3101a-h | S9 | L5 | M22 |
| E3102a-h | S9 | L6 | M22 |
| E3103a-h | S9 | L7 | M22 |
| E3104a-h | S9 | L8 | M22 |
| E3105a-h | S9 | L9 | M22 |
| E3106a-h | S10 | L1 | M22 |
| E3107a-h | S10 | L2 | M22 |
| E3108a-h | S10 | L3 | M22 |
| E3109a-h | S10 | L4 | M22 |
| E3110a-h | S10 | L5 | M22 |
| E3111a-h | S10 | L6 | M22 |
| E3112a-h | S10 | L7 | M22 |
| E3113a-h | S10 | L8 | M22 |
| E3114a-h | S10 | L9 | M22 |
| E3115a-h | S11 | L1 | M22 |
| E3116a-h | S11 | L2 | M22 |
| E3117a-h | S11 | L3 | M22 |
| E3118a-h | S11 | L4 | M22 |
| E3119a-h | S11 | L5 | M22 |
| E3120a-h | S11 | L6 | M22 |
| E3121a-h | S11 | L7 | M22 |
| E3122a-h | S11 | L8 | M22 |
| E3123a-h | S11 | L9 | M22 |
| E3124a-h | S12 | L1 | M22 |
| E3125a-h | S12 | L2 | M22 |
| E3126a-h | S12 | L3 | M22 |
| E3127a-h | S12 | L4 | M22 |
| E3128a-h | S12 | L5 | M22 |
| E3129a-h | S12 | L6 | M22 |
| E3130a-h | S12 | L7 | M22 |
| E3131a-h | S12 | L8 | M22 |
| E3132a-h | S12 | L9 | M22 |
| E3133a-h | S13 | L1 | M22 |
| E3134a-h | S13 | L2 | M22 |
| E3135a-h | S13 | L3 | M22 |
| E3136a-h | S13 | L4 | M22 |
| E3137a-h | S13 | L5 | M22 |
| E3138a-h | S13 | L6 | M22 |
| E3139a-h | S13 | L7 | M22 |
| E3140a-h | S13 | L8 | M22 |
| E3141a-h | S13 | L9 | M22 |
| E3142a-h | S14 | L1 | M22 |
| E3143a-h | S14 | L2 | M22 |
| E3144a-h | S14 | L3 | M22 |
| E3145a-h | S14 | L4 | M22 |
| E3146a-h | S14 | L5 | M22 |
| E3147a-h | S14 | L6 | M22 |
| E3148a-h | S14 | L7 | M22 |
| E3149a-h | S14 | L8 | M22 |
| E3150a-h | S14 | L9 | M22 |
| E3151a-h | S15 | L1 | M22 |
| E3152a-h | S15 | L2 | M22 |
| E3153a-h | S15 | L3 | M22 |
| E3154a-h | S15 | L4 | M22 |
| E3155a-h | S15 | L5 | M22 |
| E3156a-h | S15 | L6 | M22 |
| E3157a-h | S15 | L7 | M22 |
| E3158a-h | S15 | L8 | M22 |
| E3159a-h | S15 | L9 | M22 |
| E3160a-h | S16 | L1 | M22 |
| E3161a-h | S16 | L2 | M22 |
| E3162a-h | S16 | L3 | M22 |
| E3163a-h | S16 | L4 | M22 |
| E3164a-h | S16 | L5 | M22 |
| E3165a-h | S16 | L6 | M22 |
| E3166a-h | S16 | L7 | M22 |
| E3167a-h | S16 | L8 | M22 |
| E3168a-h | S16 | L9 | M22 |

### 3. SYNTHESIS OF THE COMPOUNDS OF THE INVENTION

In another aspect, the invention provides methods for making the compounds of the invention. The following schemes depict some exemplary chemistry available for synthesizing compounds of the invention. It will be appreciated, however, that the desired compounds may be synthesized using other alternative chemistries known in the art.

Scheme 1 illustrates the synthesis of oxazolidinones substituted at C-5 with 1,2,3-triazolylmethyl derivatives. Isocyanates 14 can react with lithium bromide and glycidyl butyrate at elevated temperature to produce oxazolidinone intermediates of type 15 (Gregory et al. (1989) J. MED. CHEM. 32: 1673). Hydrolysis of the resulting butyrate ester of compound 15 produces alcohol 17. Alcohol 17 can also be synthesized from carbamates such as the benzyl carbamate 16. The carbamate nitrogen of compound 16 then is deprotonated, and alkylated with glycidyl butyrate to produce (after in situ hydrolysis of the butyl ester) hydroxymethyl derivative 17. While the R enantiomer depicted throughout Scheme 1 generally is the most biologically useful derivative for antibacterial agents, it is contemplated that compounds derived from either the R or the S enantiomer, or any mixture of R and S enantiomers, may be useful in the practice of the invention.

Alcohols 17 can be converted to useful intermediates such as mesylates 18a (by treatment with methanesulfonyl chloride and triethylamine in an appropriate solvent) and azide 19 (by subsequent displacement of the mesylate by sodium azide in DMF). Azide 19 can also be produced from tosylate 18b (or a brosylate or nosylate), or an alkyl halide of type 18c (made from alcohol 17 via methods known to those skilled in the art). Azide 19 can be heated in the presence of substituted acetylenes 20 to produce C-5 substituted 1,2,3-triazolylmethyl oxazolidinone derivatives of type 21 and 22. It is to be understood that alternative chemical conditions could be employed by those skilled in the art to effect this transformation.

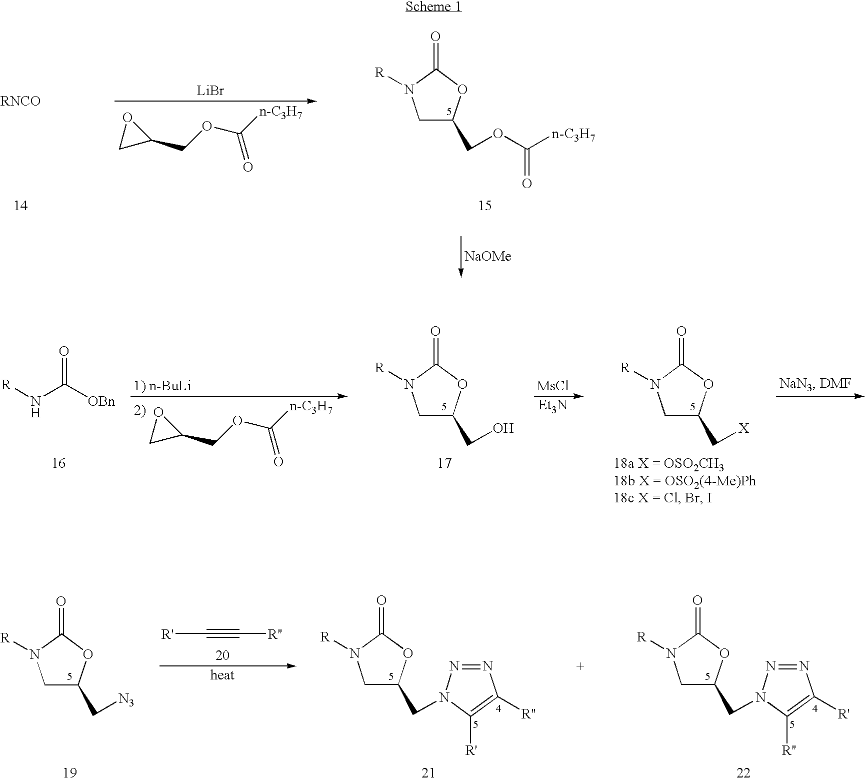

It is understood that unsymmetrical acetylene derivatives can react to produce a mixture of regioisomeric cycloaddition products, represented by 21 and 22, and that the reaction conditions can be adjusted by processes known to those skilled in the art to produce more selectively one regioisomer or the other. For example, Scheme 2 depicts the reaction of mono-substituted acetylene 23 with azide 19 to produce two regioisomeric triazoles, 24 and 25. The major isomer is most often the anti isomer 24 since the reaction leading to this product proceeds at a faster rate. Under certain circumstances, the more sterically disfavored syn isomer is also formed, but at an appreciably diminished rate. The addition of copper(I)iodide is a useful additive for this reaction, and often leads to increased proportions of the major “anti” adduct 24 (Tomoe, C. W. et al. (2002) J. ORG. CHEM. 67: 3057). Increased proportions of the minor isomer 25 may be produced by minor modification of the reaction scheme. Azide 19 can react with the trimethylsilyl substituted acetylene 26 to produce the anti isomer 27 and the syn isomer 28. Desilylation with tetrabutylammonium fluoride can produce triazole 24 and 25, with increased proportions of 25 obtainable from the more abundant precursor triazole 27.

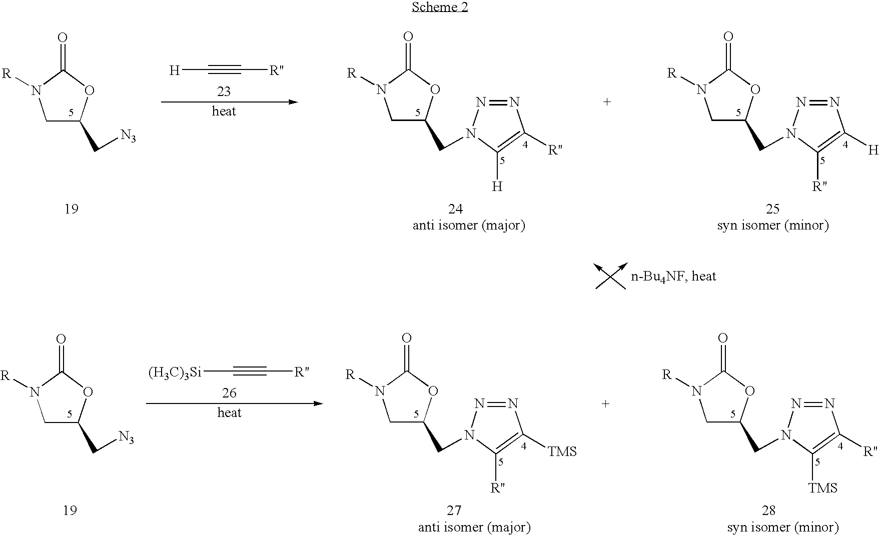

An alternate approach toward the synthesis of some of the compounds of the present invention is shown in Scheme 3a. Aromatic halide 29, when activated, can react with the anion derived from treatment of carbamate 33 with an appropriate base to produce 3-aryl substituted oxazolidinone derivatives 31 via nucleophilic aromatic substitution. Suitable bases include, for example, n-BuLi, LiN(Si(CH₃)₃), and NaH. Carbamate 33 can be synthesized by exposure of 32 to carbonyldiimidazole in DMF, followed by in situ silylation of the hydroxymethyl group of the initial product with an appropriate silyl chloride. Desilylation of derivatives of type 31 produces alcohols 17 that can be converted to the targets of the present invention by the processes described within the schemes.

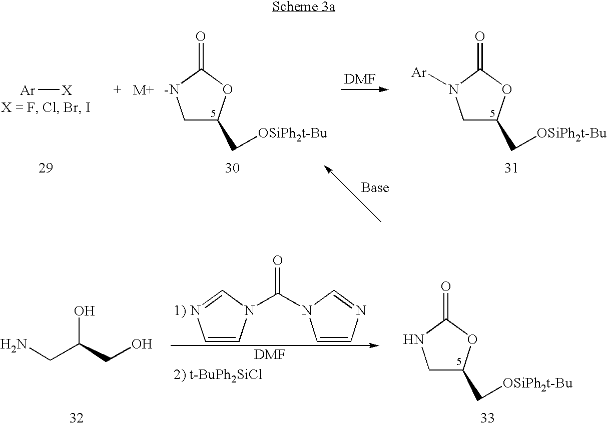

Erythromycin, as will be noted from the formula below, comprises three cyclic fragments. These fragments are referred to respectively as cladinose, desosamine and erythronolide. The naturally occurring compound erythromycin and most of its useful synthetic derivatives have the sugar desosamine attached to the C-5 oxygen of the macrolide ring. Compounds of the present invention possess an additional oxygen substituent at the 4′ position of the desosamine, i.e., they possess the sugar myaminose at the C-5 position in place of desosamine. In the present invention, all substitution takes place at the 4′ position of the desosamine moiety. Erythromycin possessing this alternate sugar was first described in 1969 in U.S. Pat. No. 3,629,232.

The first step in preparing the compounds of this invention is to prepare 4′hydroxyerthromycin. A preparative scheme for obtaining the 4′-hydroxyerthromycin is set forth in U.S. patent application Ser. No. 807,444, filed Mar. 14, 1969, and now abandoned.

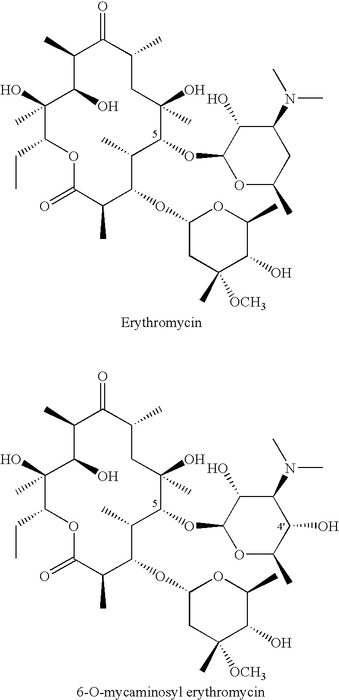

6-O-mycaminosyl-erythromycin has very similar chemical reactivity to erythromycin itself and, therefore, may be treated according to known methodology practiced on erythromycin to produce numerous useful analogs, including, for example: 6-O-mycaminosyl azithromycin, (34a), 6-O-mycaminosyl clarithromycin (34b), and 6-O-mycaminosyl clarithromycin 3-ketolide. (34c).

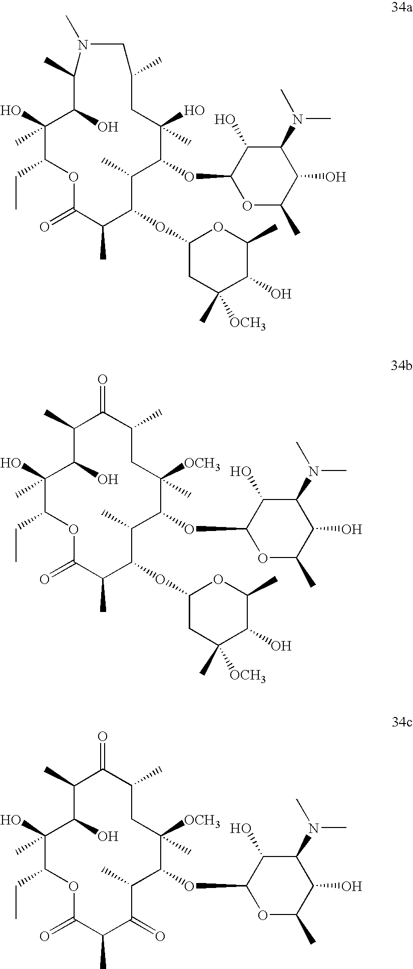

Compounds 34a, 34b, and 34c can be produced from 6-mycaminosyl erythromycin using the procedures described in U.S. Pat. Nos. 6,013,778, 5,852,180, and 5,444,051, respectively.

Secondary alcohols (or cycloalkyl alcohols) can be alkylated with electrophiles having an alkyne connected by a variable bond or linker to a carbon bearing a leaving group, for example, a halide or a sulfonate group 35, to produce ethers of type 36.

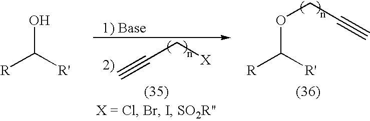

It is necessary to alkylate the 4′-hydroxyl group of the mycaminose sugar to produce compounds of the present invention from 3-mycamynosyl erythromycin or its derivatives. This is accomplished as presented in Scheme 3b. Briefly, the 2′ and 4′ hydroxyl groups of 3-mycaminosyl erythromycin can be selectively acylated by acid anhydrides in the absence of added base without causing reaction of the other hydroxyl groups of the molecule (e.g. 4″-OH, 11-OH, and 12-OH). This selectivity is possible because of the influence of the adjacent tertiary amine at the 3′ position. The remaining hydroxy groups are then protected for instance as their trimethylsilyl ethers. The acyl groups on the 2′ and 4′ hydroxyl groups are then removed selectively under mild conditions and the 4′ hydroxyl group is alkylated. Reaction of either the 4′ or 2′ oxygen without also affecting the other is typically difficult. The schemes shown below rely on the physical separation of the regioisomers obtained after such reactions when it is desired to have only the 4′ hydroxyl group substituted. Though not always explicitly shown, it is to be understood that the reaction conditions employed can cause reaction at both the 2′ and 4′ hydroxyl groups and that the desired 4′-substituted product is separated from other products in the crude reaction mixture.

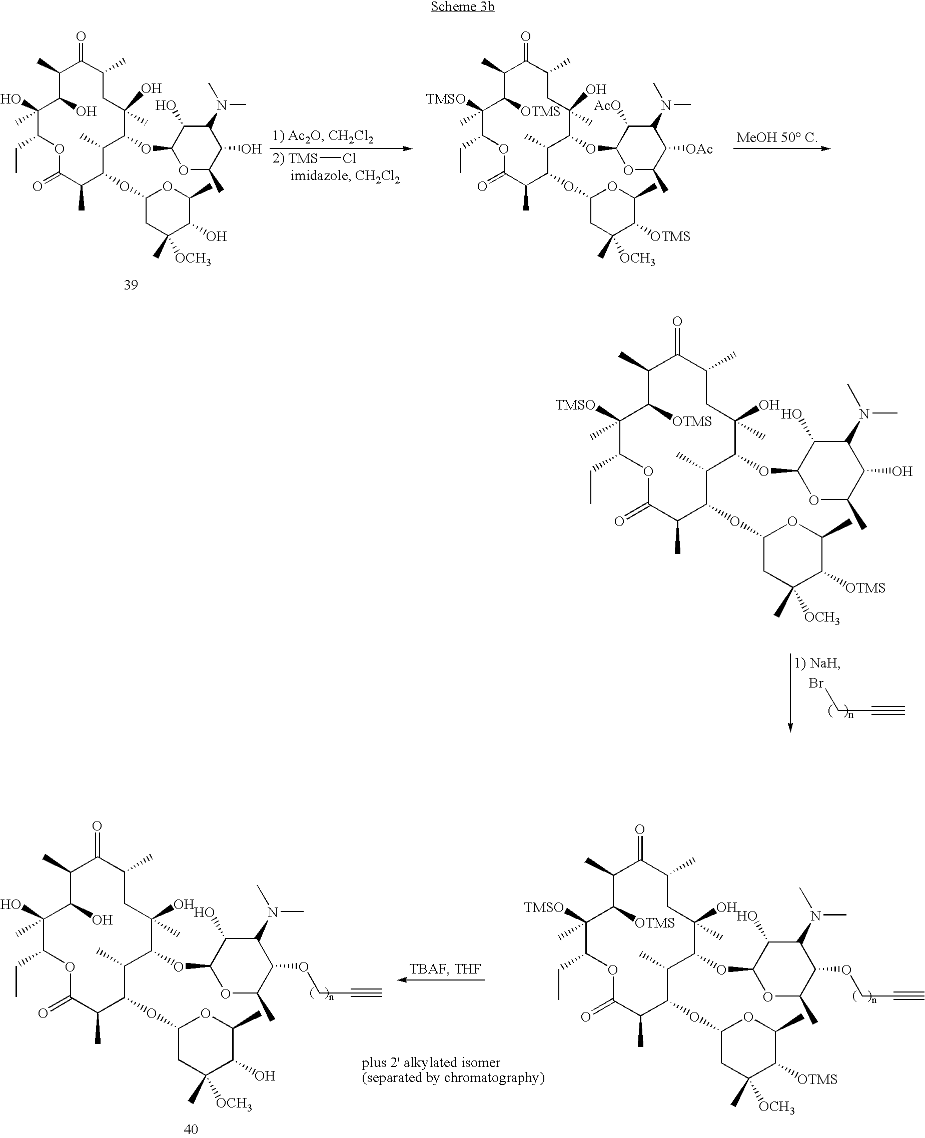

In the present case, it is necessary to protect other hydroxyl moieties in 6-mycaminosyl erythromycin from reaction. One method of accomplishing this end is presented in Scheme 3b. Since the 2′ and 4′ hydroxyl groups are the most reactive toward acylation, they are first selectively protected as esters (i.e. acetate, propionate, benzoate, trifluoroacetate etc.) by reaction with an excess of a suitable acid anhydride in an inert solvent. The remaining reactive hydroxy groups are then protected as their silyl ethers, for example, trimethyl silyl, triethyl silyl, or tert-butyldimethyl silyl ether. The 6 hydroxyl moiety is sterically hindered and does not normally react under the conditions used in the schemes. The acyl protecting groups on the 2′ and 4′ oxygens can subsequently be removed under conditions that do not affect the silyl ethers, e.g. basic hydrolysis, and methanolysis. With the 4″, 11, and 12 hydroxy groups thus protected, selective alkylation the 4′ oxygen can be achieved under standard alkylating conditions. Many other protecting groups can be successfully employed to accomplish a similar outcome. See, e.g., T. H. Greene and P. G. M. Wuts (1999) PROTECTIVE GROUPS IN ORGANIC SYNTHESIS, 3rd edition, John Wiley & Sons, New York.

Furthermore, it is understood that, given appropriate reaction conditions known to those skilled in the art, any similarly substituted macrolide antibacterial agent (naturally occurring, semi-synthetic or synthesized) is capable of serving as starting material for the processes depicted in Scheme 3b. The substituted alkynes 40 thereby obtained can be used in cycloaddition reactions with azides to yield triazole-linked target compounds.

Scheme 4 illustrates the synthesis of compounds of the present invention that contain extra keto groups in the alkyl link between the 5-membered heterocyclic ring and the macrolide moiety. Azides 19 can react with propiolate esters to produce the ester-substituted products. It is to be understood that mixtures of regioisomeric cycloadducts may form in this reaction, however, only the anti adduct is depicted in Scheme 4b. Hydrolysis of the ester yields the acid, which can be converted using known chemistry (Ramtohul et al. (2000) J. ORG. CHEM. 67: 3169) to the bromoacetyl triazole. Heating this bromoacetyl derivative with 39 (or a suitably protected version of 39) can yield products that contain a keto link with one methylene group between the ketone and the macrolide group. The bromoacetyl intermediate can be converted via lithio-dithiane chemistry, subsequent hydrolysis, and reduction to an alcohol. The tosylate (or halide) of this alcohol can be made, and this electrophile can be used to alkylate 39 to give products with two methylene groups between the ketone and the macrolide group.

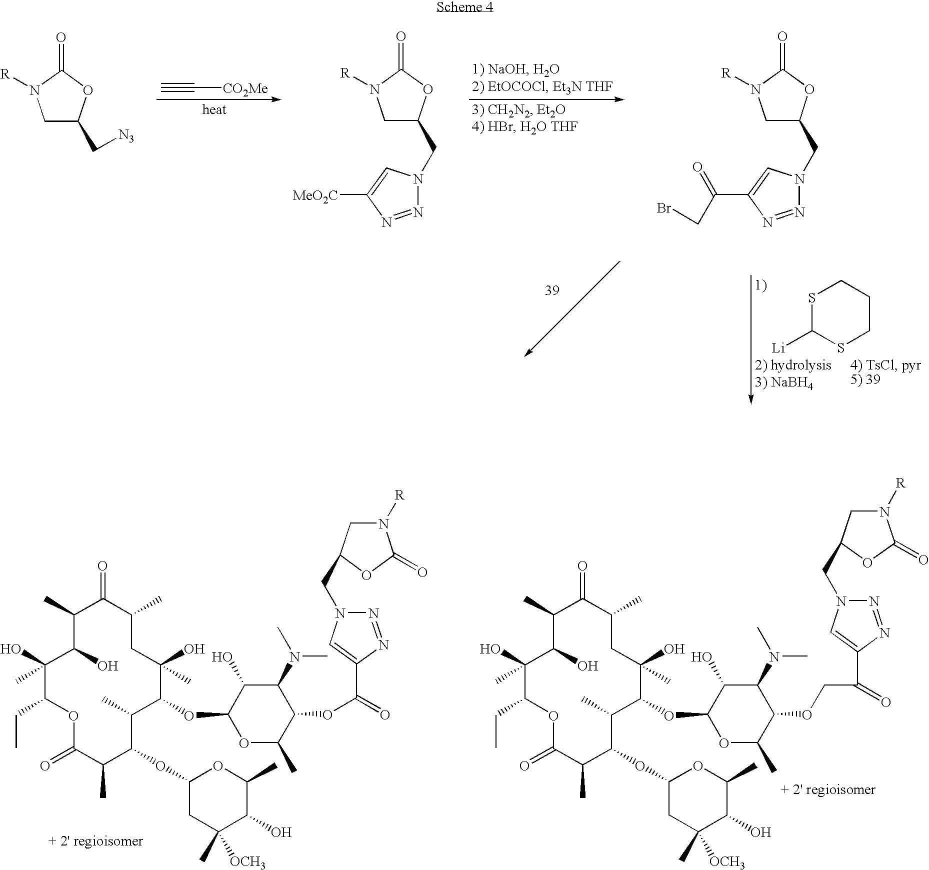

Scheme 5 illustrates another method to synthesize regioisomeric triazole-linked derivatives of the invention. Carbon-linked triazole derivatives of type 44 and 45 can be produced by first displacing a leaving group, for example, a sulfonate or a halide, from electrophiles 18a-c, with either lithium acetylide 41a or lithium trimethylsilylacetylide 41b to produce alkynes 42a or 42b, respectively. The cycloaddition reaction of alkynes 42 with appropriate azides 43 can yield regioisomeric triazoles 44 and 45. (It will be understood that alternative chemical conditions may be employed to produce compounds 44 and 45 such as the use of copper(I)iodide instead of heat.)

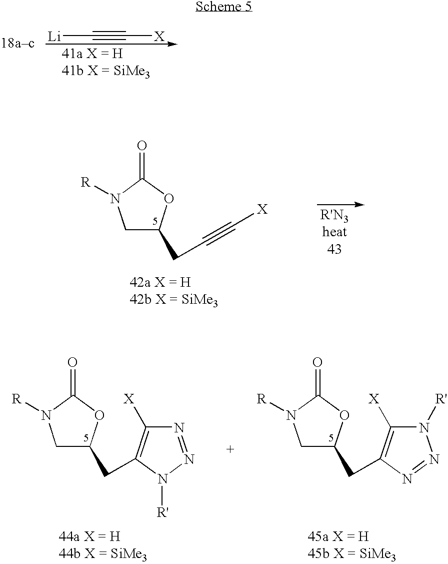

A specific example of the utility of the chemistry expressed in Scheme 5 is shown in Scheme 6. 6-Mycaminosyl-erythromycin derivative 39 (or a suitably protected derivative thereof) can be alkylated with a protected bromoalcohol, and the alcohol function of the product converted to a leaving group such as a tosylate. The tosylate can be displaced with sodium azide to yield azide 46. Cycloadditon of 46 and alkyne 42a can produce final targets of type 47. Alternative alkylsulfonates or halides can be used as the starting material for the synthesis of azide 46 (i.e., different leaving groups). Other mycaminose-containing macrolide entities can be used in place of the 6-mycaminosyl-erythromycin derivative 39 to produce a variety of alternative products.

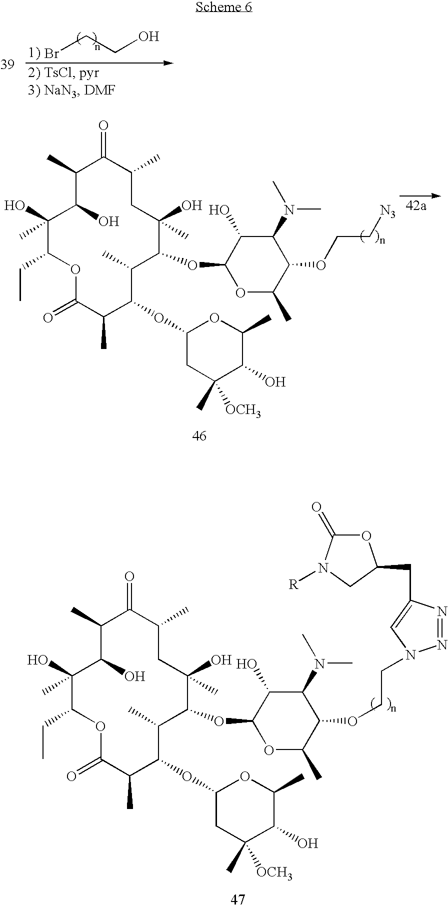

Another method that can be used to synthesize carbon-linked triazole derivatives of type 47 is illustrated in Scheme 7. Alkyne 42a can react with trimethylsilylazide (or with sodium azide, ammonium chloride and copper(I)iodide, or other conditions known in the art) to produce two possible regioisomeric products, triazoles 48 and 49. Either of these (or the mixture) can be desilylated with n-Bu₄NF to produce triazole 50. Des-methyl erythromycin derivative 39 (or an alternate 4′-hydroxy macrolide derivative) can be converted to tosylate 51 (or another sulfonate or halide electrophile), and then the electrophile can serve to alkylate triazole 50 to produce either the N-1 substituted triazole 47, or the N-2 substituted triazole 53, or a mixture of both. In the event that a mixture is produced, both compounds may be separated from one another. It is contemplated that other macrolides may be transformed by the chemistry of Scheme 7 to produce other compounds of interest.

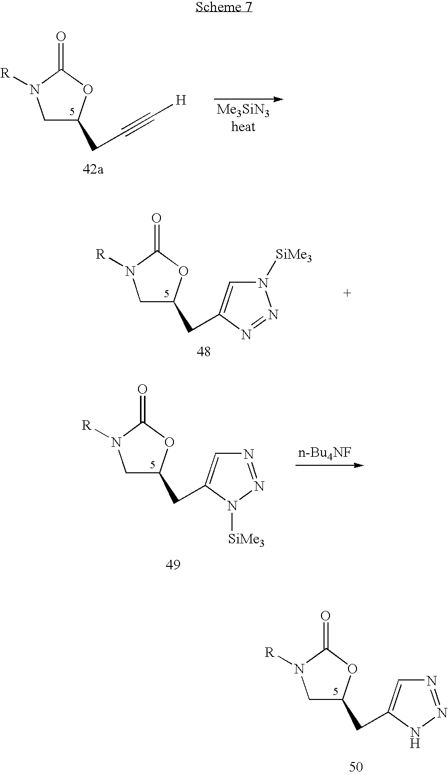

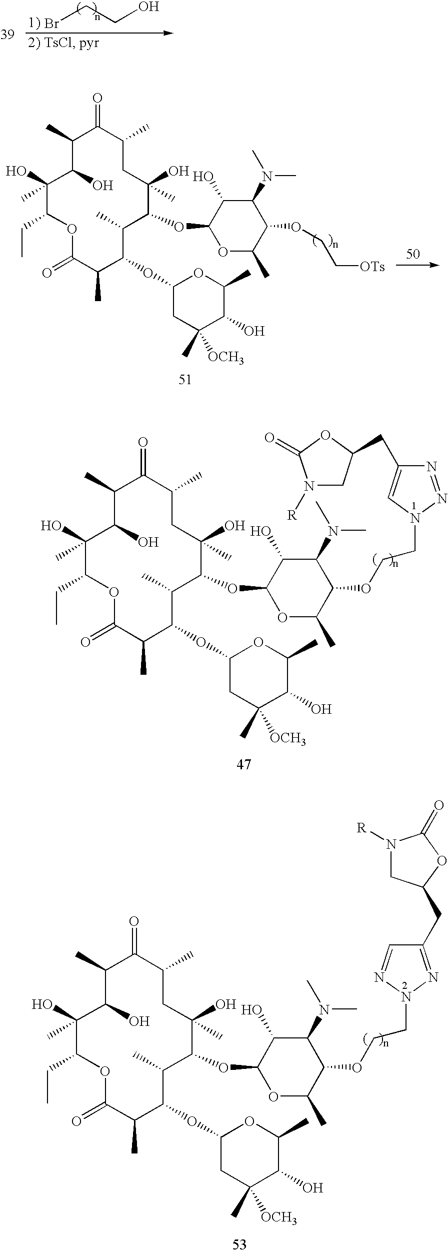

Scheme 8a illustrates the synthesis of oxazolidinones substituted at C-5 with tetrazolylmethyl derivatives. Azides of type 19 can react with nitrites 54 to produce tetrazoles of type 55 and 56. In a similar fashion to the chemistry described in Scheme 1, this reaction can yield regioisomeric cycloadducts, where the anti isomer often predominates. As an example, 4′-hydroxy erythromycin 39 can be alkylated with (ω-halo or ω-sulfonate nitrites 57 to yield nitriles. 58. These derivatives can react with azides of type 19 to produce target tetrazoles of type 59 and 60. It is to be understood that the R′ group of nitrites 54 may contain the macrolide moiety, or suitable substituted alkyl groups containing an alcohol or protected alcohol that can be converted to a leaving group prior to a final alkylation step with a macrolide. Thus, the tetrazoles 55 and 56 can be produced that have as their R′ groups alkyl chains bearing a hydroxy group that can be converted into a sulfonate or halide leaving group prior to alkylation with alcohols similar to 39 to afford products of type 59 and 60.

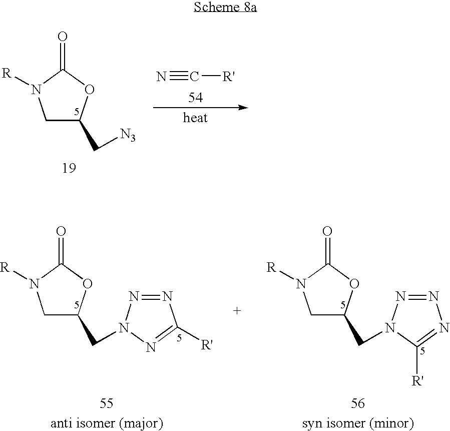

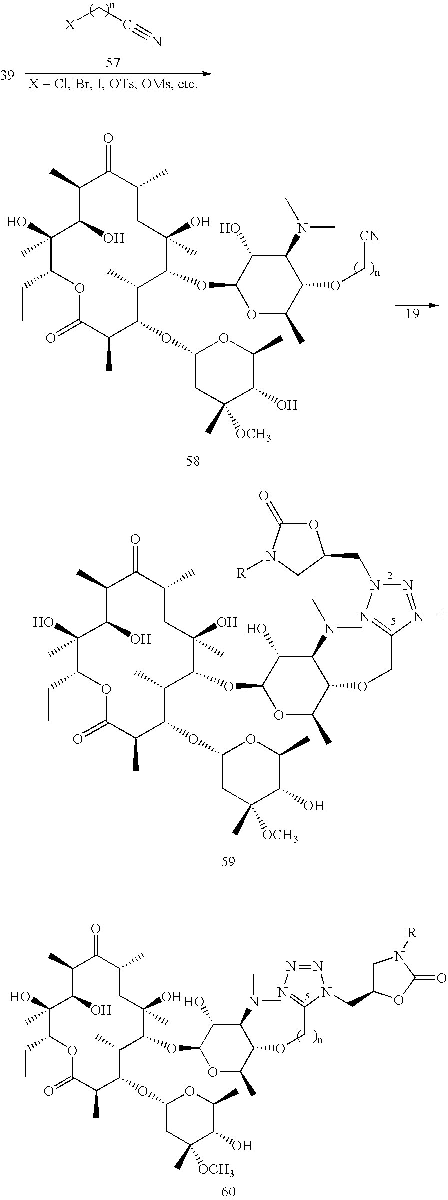

Scheme 8b depicts another strategy to synthesize tetrazoles of type 59 and 60. Azides 19 may undergo cycloaddition to functionalized nitrites of type 57a to afford tetrazole intermediates 55a and 56a. If 55a and 56a contain an appropriate electrophilic group such as a halide or sulfonate, it can react directly with macrolides of type 39 (or a suitably protected derivative thereof) to yield targets of type 59 and 60. Alternatively, silyloxy-substituted nitrites 57a may be used during the cycloaddition reaction to afford intermediates of type 55a and 56a where X is a silyloxy group. The silylether protecting group may then be removed from 55a and 56a, and the resultant alcohol converted to an appropriate electrophile (such as a halide or sulfonate) that would then be suitable for alkylation of macrolides of type 39 to give the desired targets.

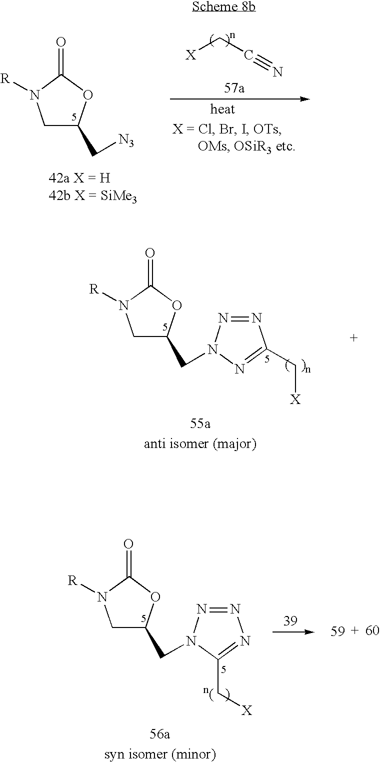

Scheme 9 illustrates one method of synthesizing pyrazole derivatives of the present invention. Known trityl-protected organolithium derivative 61 (Elguero et al. (1997) SYNTHESIS 563) can be alkylated with electrophiles of type 18a-c to produce pyrazoles of type 62. Cleavage of the trityl group can be accomplished using a variety of acidic reagents, for example, trifluoroacetic acid (TFA), to produce pyrazole 63. Alkylation of 63 with a bromoalcohol of appropriate length, followed by tosylation (or alternate sulfonation or halide formation) can produce electrophiles 64. Alkylation of 39 with 64 produces targets of type 65. The lithium anions derived from heterocycles such as 61 may optionally be converted to copper (or other metallic) derivatives to facilitate their displacement reactions with sulfonates and halides. These anions may also be allowed to react with suitably protected macrolides, such as the per-silylated derivative of 51.

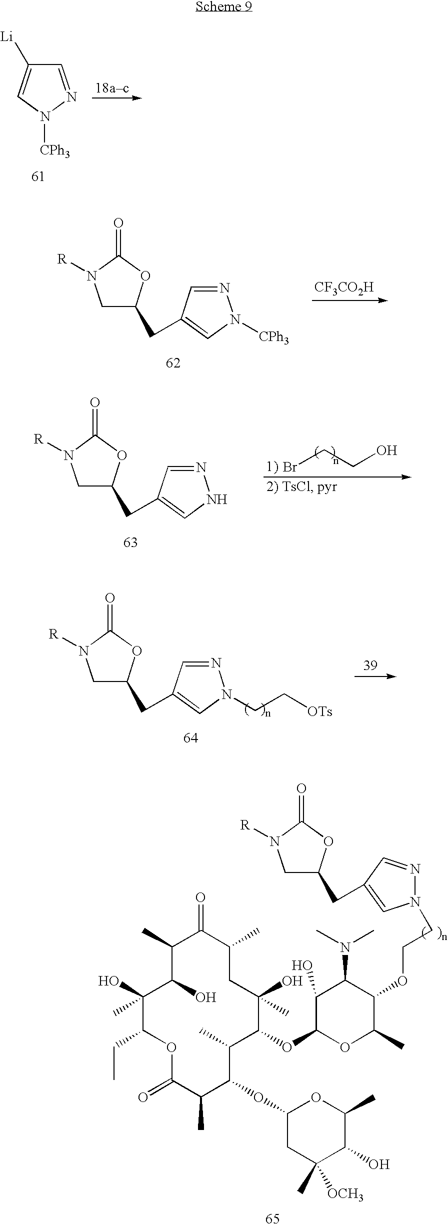

Scheme 10 depicts another method of synthesizing pyrazoles of the present invention. Anions 61 can be alkylated with a bifunctional linker of variable length such as an alkyl halide containing a silyloxy derivative. Alternatively an α,ω dihaloalkyl derivative can be used as the alkylating agent, or a mixed halo-sulfonate can be employed for this purpose. The resulting substituted pyrazoles 66 can be converted to the free pyrazoles by TFA cleavage of the triphenylmethyl protecting group. The free pyrazoles can undergo direct alkylation with electrophiles 18a-c in a suitable solvent, for example, dimethylformamide, or can be first converted via deprotonation with a suitable base, for example, sodium hydride or n-butyllithium, to the corresponding anion, if a more reactive nucleophile is required. The resultant pyrazole derivatives 67 can be desilylated and converted to tosylates 68 (if a sulfonate strategy is employed), which can serve as electrophiles for subsequent reaction with macrolide saccharides, for example, 39, to produce the resultant target 69.

Another approach to intermediates of type 67 can start with alkylation of the known dianion 70 (Hahn et al. (1991) J. HETEROCYCLIC CHEM. 28: 1189) with an appropriate bifunctional linker to produce compounds related to pyrazole 71, which can subsequently be alkylated (with or without prior deprotonation) with electrophiles 18a-c to produce intermediates 67. The n=1 derivatives in this series can be synthesized by trapping compound 61 with DMF to produce the corresponding aldehyde, and then reduction to the alcohol. Alternatively, methoxymethyl (MOM) chloride or bromide can serve as the alkylating reagent for 61, and hydrolysis of the trityl and MOM groups of the product would yield 4-hydroxymethyl-1,2-pyrazole. The dianion of this pyrazole can be alkylated on nitrogen to produce an alcohol that serves as the precursor for an n=1 tosylate (or other leaving group).

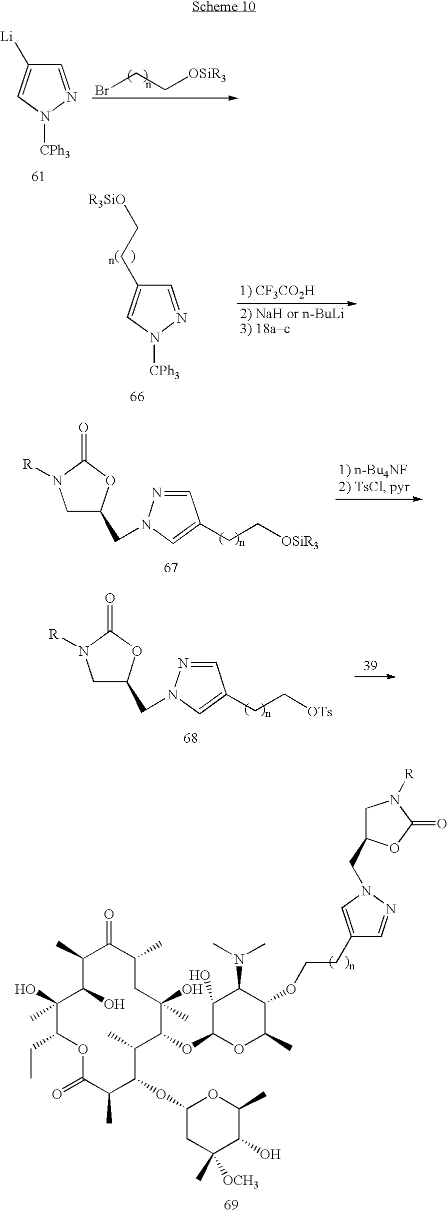

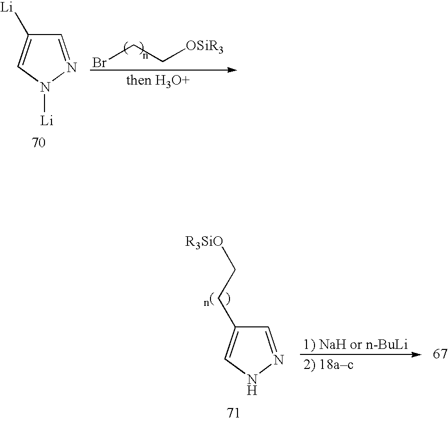

Scheme 11 shows an alternate approach for synthesizing pyrazole derivatives of type 69. Alkylation of the anion of a β-dicarbonyl system with appropriate electrophiles similar to tosylate 51 can yield (in the specific example of β-dicarbonyl derivative 72a) products of type 73. Treatment of these intermediates with hydrazine can produce pyrazoles of type 74. Direct alkylation of 74 with electrophiles 18a-c can proceed to produce targets 69. Alternatively, the hydroxyl residues of 74 (and other sensitive functional groups of other macrolide derivatives such as intermediates 39 and 51) can be protected with suitable protecting groups (such as those highlighted in Greene, T. W. and Wuts, P. G. M. supra), and the hydrogen atom on the nitrogen atom of the pyrazole derivative deprotonated with a suitable base, for example, sodium hydride or n-butyllithium. The resulting anion can then be alkylated with electrophiles 18a-c, and the resulting product deprotected to produce targets 69. The use of protecting groups well known to those skilled in the art for the macrolide portions of these intermediates may be required for many of the subsequent reactions shown in the schemes below that involve heteroaryl anion alkylations.

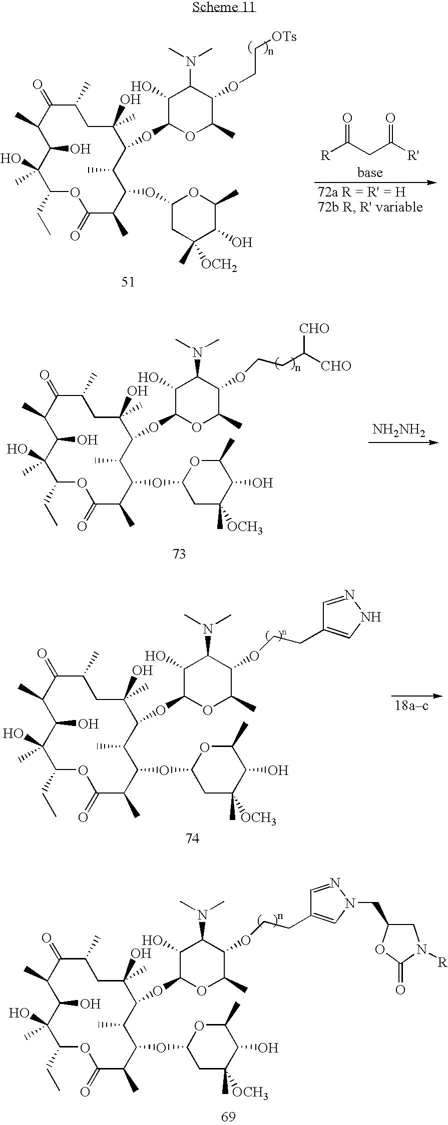

Scheme 12 exemplifies a synthesis of imidazoles of the present invention. The known dianion 75 (Katritzky et al. (1989) J. CHEM. SOC. PERKIN TRANS. 1: 1139) can react with electrophiles 18a-c to produce after protic work-up imidazoles of type 76. Direct alkylation of 76 by heating with electrophiles related to 51 in an appropriate organic solvent can yield 1,4-disubstituted imidazoles 77. Alternatively, the imidazole anion formed via deprotonation of the imidazole hydrogen atom of 76 with a suitable base and then alkylation with 51 can also produce 77.

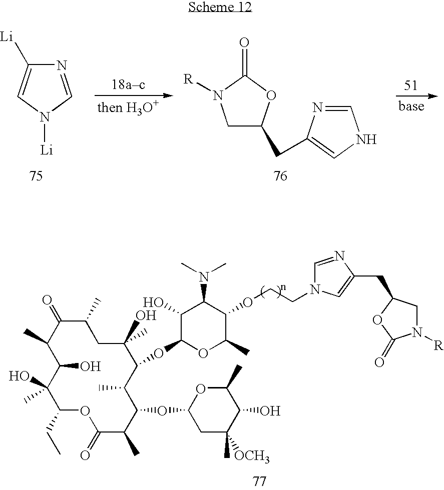

Scheme 13 illustrates another synthesis of imidazoles of the present invention. 4-Bromoimidazole can be deprotonated using, for example, sodium hydride or lithium diisopropylamide, or another suitable organic base, to give anion 78 (or the corresponding lithio derivative). Alkylation of 78 with 18a-c can yield bromoimidazole 79 which can then be subjected to metal-halogen exchange and alkylated with 51 (or a suitably protected derivative of 51) to produce isomeric 1,4-disubstituted imidazoles 80.

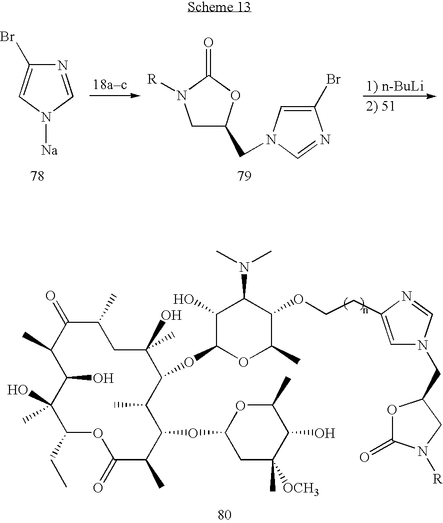

Scheme 14 depicts chemistry suitable for the synthesis of other target imidazole derivatives. The silylethoxymethyl (SEM) protected imidazole 81 can be lithiated at C-2 (Shapiro et al. (1995) HETEROCYCLES 41: 215) and can react with electrophiles 18a-c to produce imidazole intermediates 82. Lithiation of imidazole intermediates 82 at C-4 of the imidazole, followed by alkylation with electrophiles of type 51 (or a suitably protected version such as the per-silylated derivative), and then deprotection of the SEM can produce imidazoles 83.

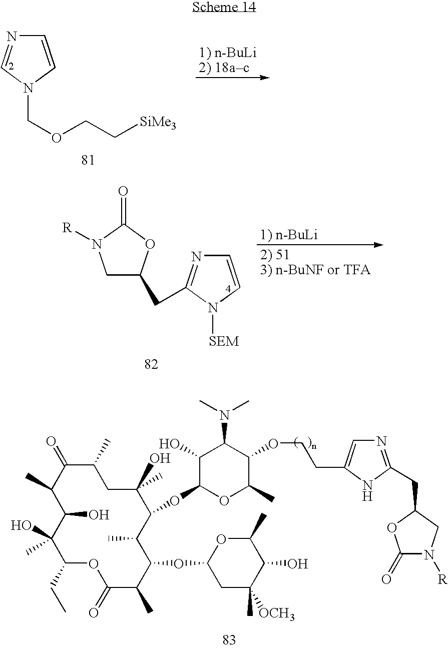

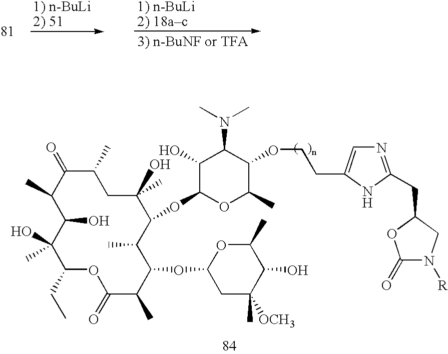

Scheme 15 shows how tosylmethyl isocyanide can be used to make imidazoles of the present invention (Vanelle et al. (2000) EUR. J. MED. CHEM. 35: 157; Horne et al. (1994) HETEROCYCLES 39: 139). Alcohols 17 can be oxidized to produce aldehydes 85 using an appropriate agent such as the Dess-Martin periodinane, or oxalyl chloride/dimethylsulfoxide/triethylamine (Swern oxidation). A variety of chromium complexes can also be used for this oxidation, including, for example, pyridinium dichromate (PDC), pyridinium chlorochromate (PCC), chromium trioxide, and tetrapropylammonium perruthenate. Wittig homologation of 85 can provide aldehyde 86, which can then be converted by tosylmethyl isocyanide to produce intermediate 87. The reaction of 87 with 89 (formed via alkylation of alcohols 39 with bromoalkyl phthalimides 88 (followed by hydrazine cleavage) or reduction of azides 46) can produce imidazoles 77.

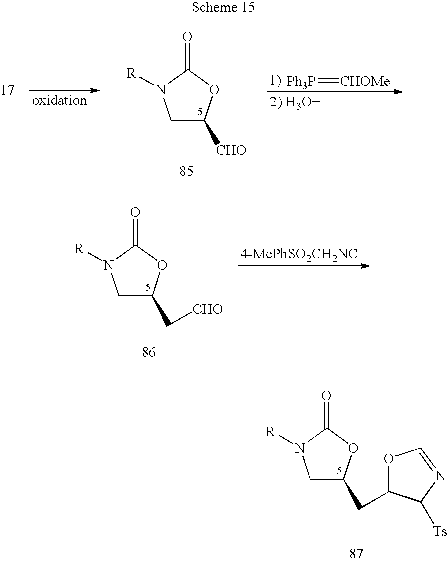

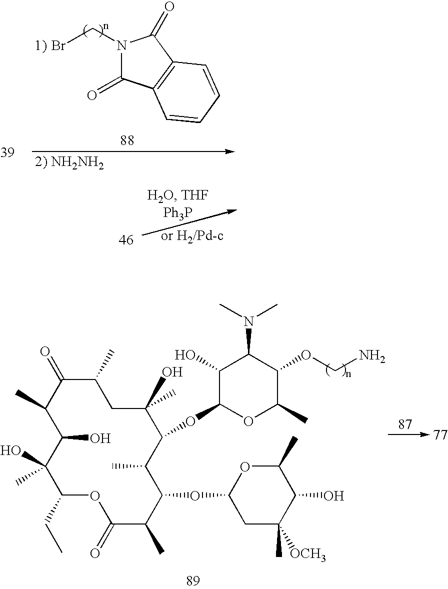

Scheme 16 delineates how 1,3 thiazole and 1,3 oxazole derivatives of the present invention can be synthesized. Known dibromo thiazoles and oxazoles 90a and 90b can be selectively metallated at C-2 and alkylated with electrophiles 18a-c to produce intermediates 91a and 91b (Pinkerton et al. (1972) J. HETEROCYCLIC CHEMISTRY 9: 67). Transmetallation with zinc chloride can be employed in the case of the oxazole anion if the anion displays any tendency to ring open prior to its reaction with certain electrophiles. The bromo azoles 91 can be metallated to form the corresponding anion which can undergo alkylation with sulfonates 51 for the related halides) to produce the final targets 92. Reordering of the sequence of electrophiles in this process permits access to the isomeric thiazoles and oxazoles 93.

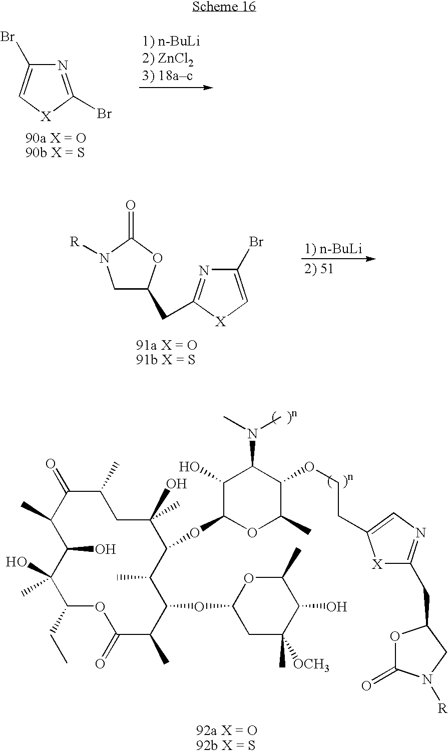

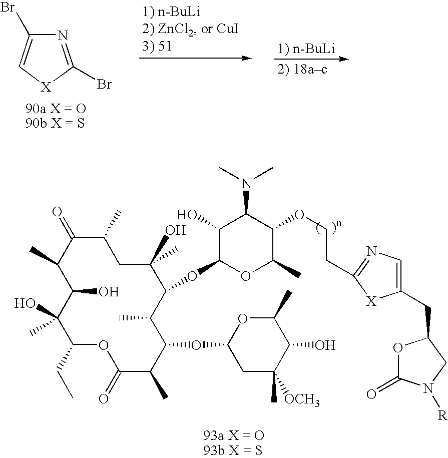

Scheme 17 shows the synthesis of 2,5 disubstituted furan and thiophene derivatives of the invention. Commercially available dibromofuran 94a and dibromothiophene 94b can be monolithiated (Cherioux et al. (2001) ADVANCED FUNCTIONAL MATERIALS 11: 305) and alkylated with electrophiles 18a-c. The monobromo intermediates obtained from this reaction can be lithiated again and then alkylated with electrophiles of type 51 (or a protected version of 51) to produce the final targets 95.

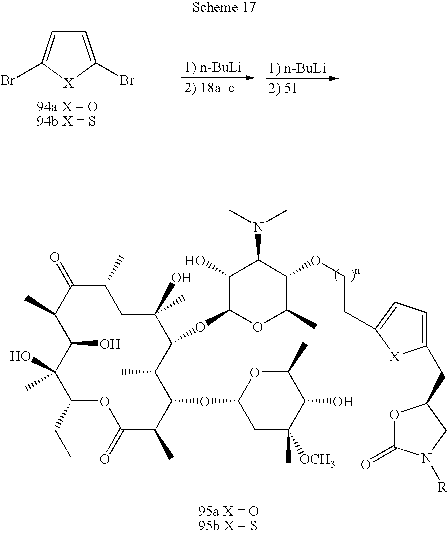

Scheme 18 depicts the synthesis of 2,4 disubstituted furan and thiophene derivatives of the invention. Commercially available furan aldehyde 96a, and the known thiophene aldehyde 96b, can be reduced to the corresponding alcohols and the resulting alcohols converted to a leaving group such as tosylates 97. Alternate sulfonates and halides can be synthesized and used in this fashion. The tosylates 97 can alkylate alcohol 39 (or a protected version thereof), and the heteroaryl bromide can be converted to a suitable organometallic agent (by reagents such as n-BuLi, or i-Pr₂Mg/CuCN). This intermediate organometallic agent can be alkylated with electrophiles 18a-c to produce targets of type 98 where n=1. As the scheme shows, a reordering of steps can be employed involving reduction, silylation, lithiation and then initial alkylation with 18a-c. Desilylation of the alkylation product, followed by tosylation of the alcohol, provides an intermediate that can then be alkylated with alcohol 39 to produce targets 98. Simple homologation protocols, using the reagents depicted in Scheme 18 or others known to those skilled in the art, can convert the aldehydes 96 to longer chain tosylates such as 99 and 100. The use of these tosylates in the alkylation with 39, and subsequent metal-halogen exchange and alkylation with 18a-c, can yield compounds of type 98 where n=2 and 3. It will be appreciated that longer chain tosylates can be produced using chemistries similar to that depicted in Scheme 18, and that other bifunctional linkers can be used to produce compounds of type 98.

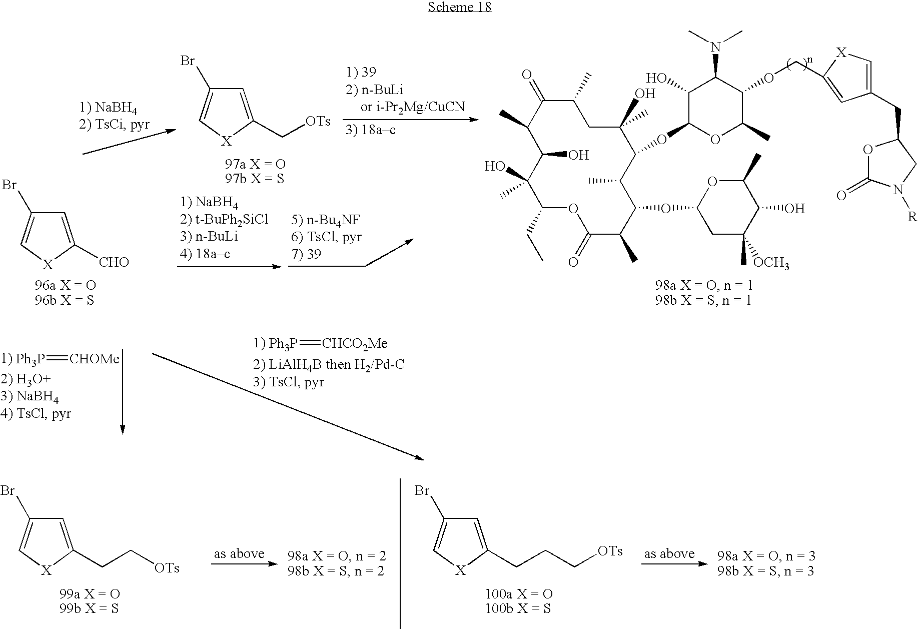

Chemistries similar to that employed above in Scheme 18 can convert known thiophene aldehyde 101 (Eras et al. (1984) J. HETEROCYCLIC CHEM. 21: 215) to produce products of type 104 (Scheme 19). The known acid 102 (Wang et al. (1996) TETRAHEDRON LETT. 52: 12137) can be converted to aldehyde 103 by reduction with, for example, borane or lithium aluminum hydride, followed by oxidation of the resultant hydroxymethyl intermediate with, for example, PDC, PCC, or another suitable reagent. Aldehyde 103 can then be converted to produce compounds of type 104.

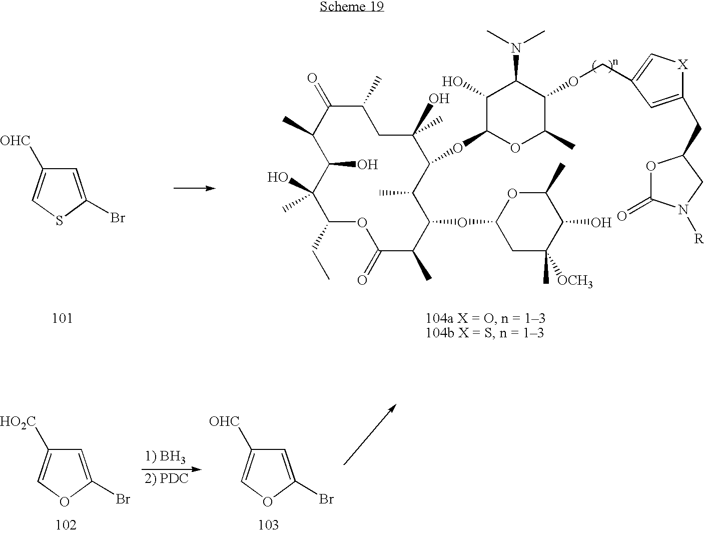

Scheme 20 illustrates the synthesis of 2,5 disubstituted pyrroles of the invention. The BOC-protected dibromopyrrole 105 can be lithiated and alkylated sequentially (Chen et al. (1987) TETRAHEDRON LETT. 28: 6025; Chen et al. (1992) ORG. SYNTH. 70:151; and Martina et al. (1991) SYNTHESIS 613), and allowed to react with electrophiles 18a-c and 51 (or a suitably protected analogue of 51) to produce, after final BOC deprotection with TFA, disubstituted pyrroles of type 106.

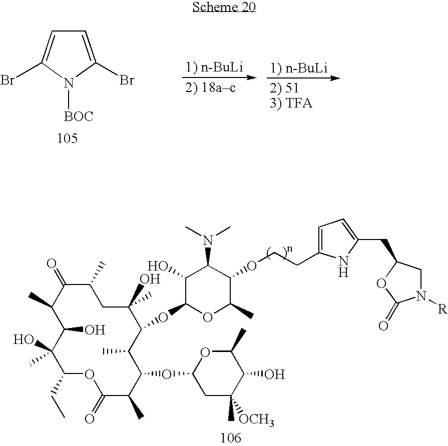

Scheme 21 shows the synthesis of 2,4 disubstituted pyrroles of the invention. Commercially available pyrrole ester 107 can be protected with a suitable protecting group, for example, the BOC group, and the ester function hydrolyzed to the corresponding acid. The resulting acid can then be reduced to the alcohol using, for example, borane to yield an alcohol that can be converted to tosylate 108. Alcohol 39 (or a suitably protected version of 39, formed for example by silylation of the other hydroxyl groups with bis-trimethylsilylacetamide or another silylating reagent) can be alkylated with tosylate 108 to produce an intermediate bromopyrrole. The bromopyrrole can then be converted to an organometallic reagent that can then react with electrophiles 18a-c. The resulting product can then be deprotected with TFA to produce pyrroles 109. The alcohol formed after borane reduction of the acid derived from 107 can then be homologated to tosylates 110 and 111 by chemistries similar to that shown below in Scheme 23. The use of these tosylates in the alkylation strategy can produce target pyrroles of type 109 where n=2 and 3.

An alternative approach is to protect the alcohol functions prior to tosylation, and perform the alkylation of the organometallic derived from the halopyrrole with 18a-c first. For example, silyloxy derivative 112 can be produced from 107, and the organometallic derivative derived from it alkylated with 18a-c to yield silyl ethers 113. Subsequent desilylation and conversion to tosylates 114 provides an electrophile that can be used in the alkylation reaction with 39. A final BOC cleavage can then give pyrroles 109. It is understood that the alcohol precursor of 112 can be homologated, using chemistries similar to that shown below in Scheme 23 and other schemes) to other alkanols that can be tosylated for further reactions with alcohol 39 (or related macrolides). Furthermore, the alcohol derived from silyl cleavage of 113 can serve as the starting material for this type of homologation efforts to produce the alkyl tosylates (or halides) required for making targets 109 where n is variable.

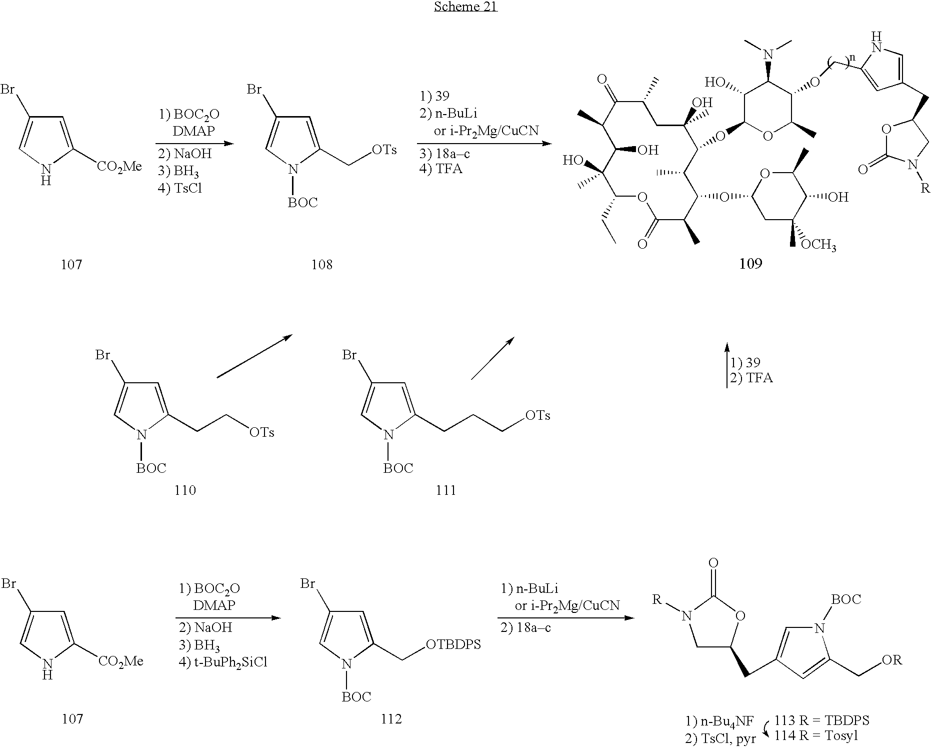

Scheme 22 shows the synthesis of isomeric 2,4 disubstituted pyrroles of the invention. Commercially available pyrrole acid 115 can be protected as the BOC derivative, and the acid function reduced to an alcohol, which can then be protected to produce the silyl ether 116. Deprotonation of 116 with n-butyllithium can occur at the 5 position of the pyrrole ring, and this anion (or that derived from transmetallation with an appropriate metal) can be alkylated with electrophiles 18a-c to produce pyrrole 117. Desilylation of 117, followed by tosylation, alkylation with 39, and TEA deprotection of the BOC group can yield pyrroles 119.

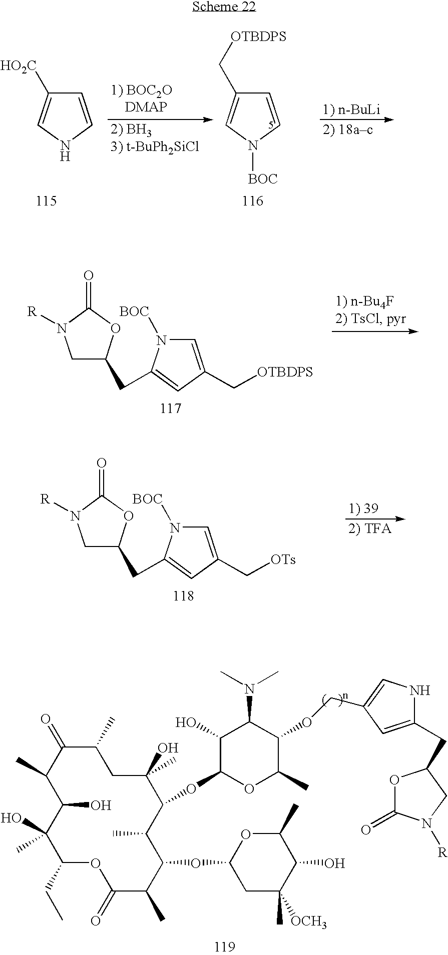

Scheme 23 illustrates the synthesis of longer chain tosylates of type 123 and 126 used to alkylate alcohols of type 39 to produce pyrroles 119. The alcohol 120 derived from protection of 115 followed by borane reduction can be oxidized to aldehyde 124. The Wittig reaction of aldehyde 124 with methoxymethyl triphenylphosphorane is followed by an acid hydrolysis step to produce the homologated aldehyde 121. Reduction and silyl protection can yield 122, which can then be deprotonated, alkylated and then converted to tosylate 123. Aldehyde 124 can undergo a Wittig reaction with carbomethoxymethyl triphenylphosphorane. The Wittig product then is reduced to an alkanol that can then be silylated to produce 125. Conversion of 125 to pyrroles 119 can then occur using the same chemistry employed to provide 119 from 122.

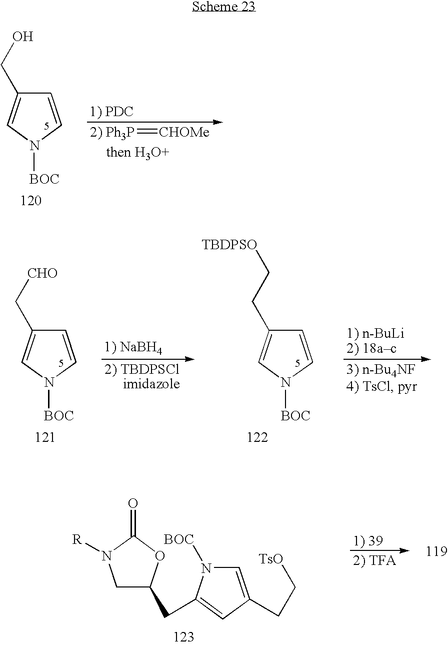

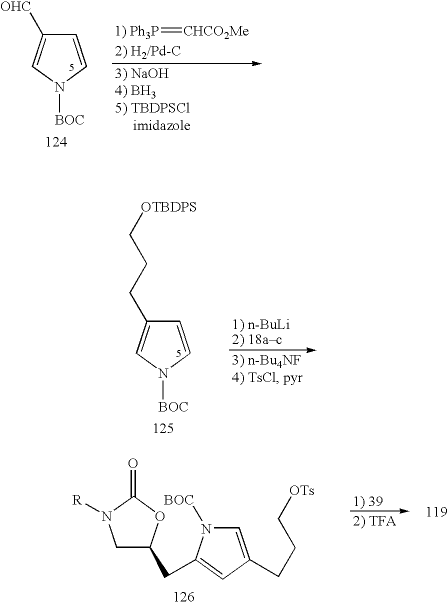

Scheme 24 shows the synthesis of 1,3 disubstituted pyrroles of the present invention. The BOC group of 116 can be cleaved to produce free pyrrole 127. Alkylation of 127 (in a suitable organic solvent such as DMF) with 18a-c can produce intermediate 128. The dianion of 3-hydroxymethylpyrrole can also be suitable for alkylation with 18a-c to produce the free hydroxy derivative of silyl ether 128. Conversion of the siloxy group to the corresponding tosylate, followed by alkylation with alcohols of type 39 can generate the target N-substituted pyrroles 129 (where n=1). In a similar fashion, the BOC pyrroles 122 and 125 can be converted to the tosylates 130 and 131. These tosylates can be used to produce pyrroles of type 129 (where n=2 and 3). It is understood that longer chain alkyl tosylates (and halides) can be produced that can undergo this chemistry to produce pyrroles 129 where n is >3.

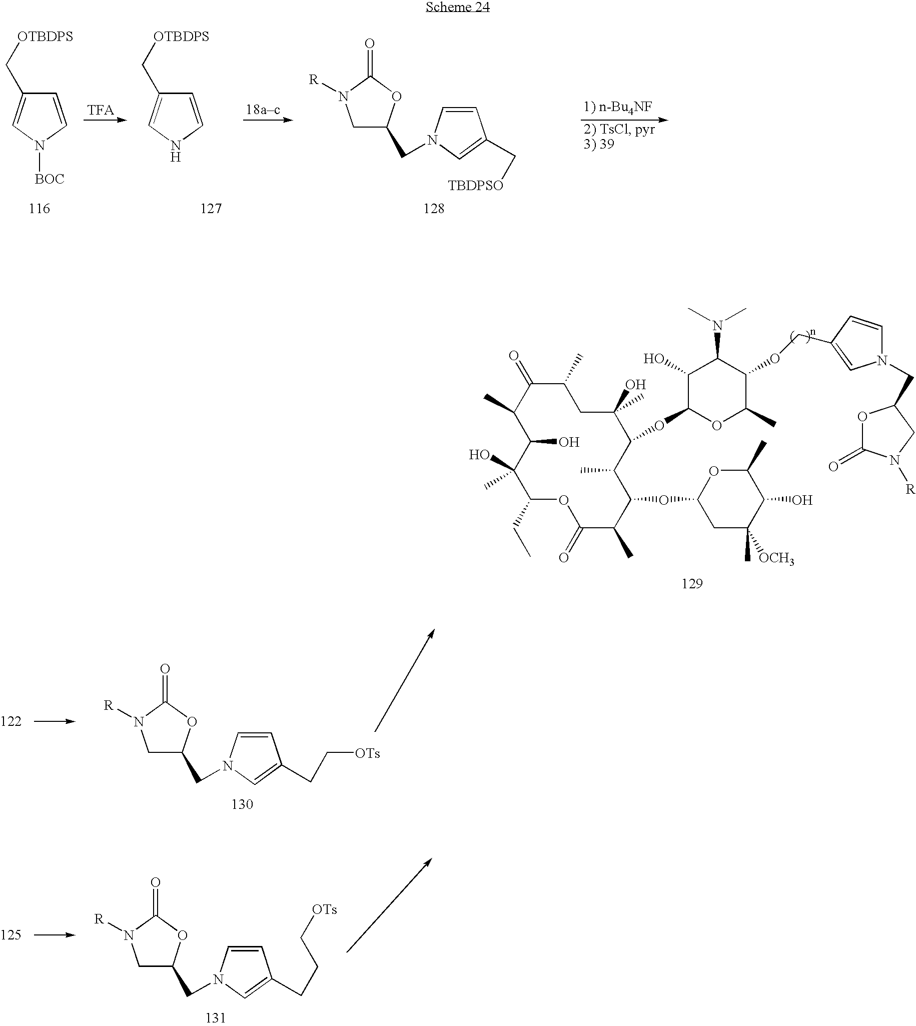

Scheme 25 illustrates the use of hydantoin-like groups as the 5-membered heterocyclic linker between the G groups and the R¹ moieties of the present invention. Electrophiles of type 18a-c can alkylate anions derived from hydantoins to produce compounds of the present invention. For example, 3-substituted hydantoins of type 132 can be purchased and treated with an appropriate base to generate the corresponding imide anion. The resulting anions can be alkylated with electrophiles similar (but not limited) to intermediates 18a-c to produce hydantoin derivatives 134. Alternatively, 1-substituted hydantoins of type 133 can be purchased or prepared, and treated with base and electrophile to yield isomeric hydantoin derivatives 135. It is understood that such hydantoins can have, for example, at optional locations, thiocarbonyl functionalities in place of the illustrated carbonyl groups. Such compounds can be prepared by treatment of the oxy-hydantoins with Lawesson's reagent, elemental sulfur, phosphorus pentasulfide, and other reagents commonly used in the art to perform this transformation.

Alternatively, such thiohydantoins can be synthesized selectively by sequential synthetic steps known in the art. The R′ group of 132 and 133 may represent a protecting group function, for example, benzyl, alkoxybenzyl, benzyloxycarbonyl, t-butoxycarbonyl, that is compatible with the alkylation step. Such a protecting group can subsequently be removed from products 134 and 135, yielding products where the R′ group is a hydrogen atom. These intermediates can be used to produce various target molecules by their treatment with base and then subsequent exposure to appropriate electrophiles.

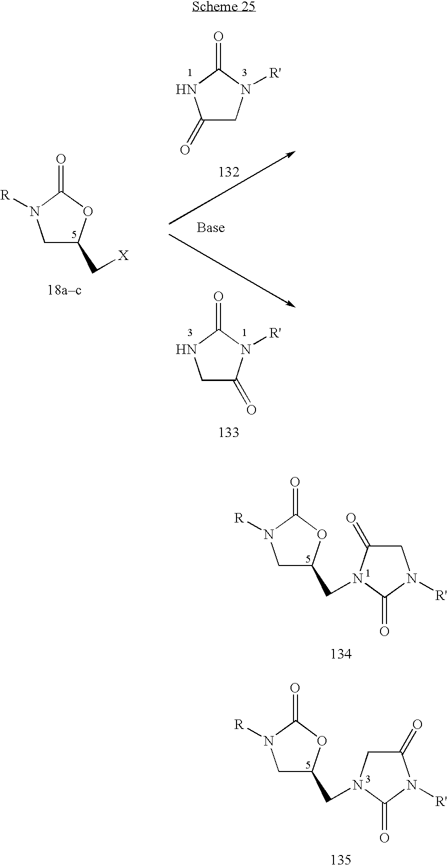

A more specific example of the synthesis of hydantoin derivatives of the present invention is depicted in Scheme 26. Hydantoin 136 can be treated with a mild organic base, for example, sodium hydride, potassium tertiary-butoxide, cesium, sodium, or potassium carbonate, to produce the N-1 substituted intermediate 137. Deprotonation of 137 with a base, for example, sodium hydride, n-butyllithium, lithium bis-trimethylsilylamide or lithium diisopropylamide, followed by alkylation with 51 (or a suitably protected derivative of 51) can yield hydantoin targets of type 138. The isomeric hydantoin derivatives of type 141 can be synthesized from 136 by initial p-methoxybenzyl (PMB) protection of the N-1 position, followed by alkylation at N-3 with 18a-c and subsequent deprotection of the PMB group with either 2,3-dichloro-3,4-dicyano-benzoquinone (DDQ) or hydrogenation will yield hydantoin intermediates 140. Subsequent alkylation of 140 with 51 can give compounds 141. Another route to produce intermediates 140 is by formation of the dianion of hydantoin 136. One equivalent of a weak base can deprotonate the N-1 position of 136. The addition of another equivalent of a strong base, for example, n-butyllithium, to the initial anion can deprotonate it again, this time at N-3. Alkylation can occur at the more reactive position N-3) to again produce hydantoins 140.

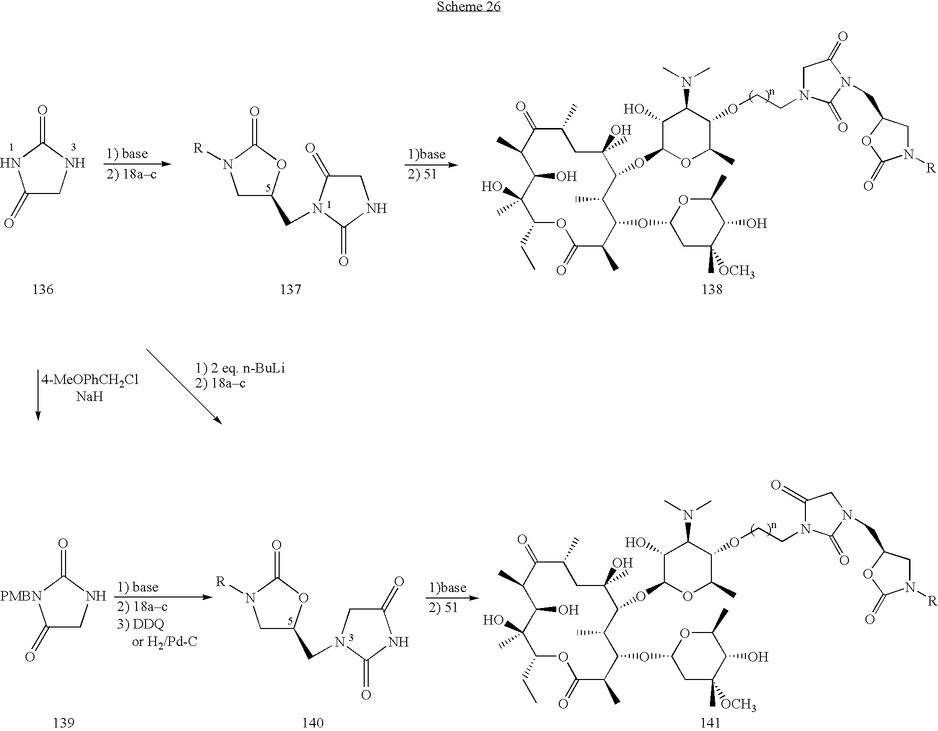

Compounds of the present invention containing an ester moiety linking the 5-membered heterocyclic ring to the macrolide can be prepared. Scheme 27 illustratates how alkynyl ester 142a or cyano ester 142b can be treated with azide 19 to yield the corresponding triazole 143a or tetrazole 143b, respecitvely.

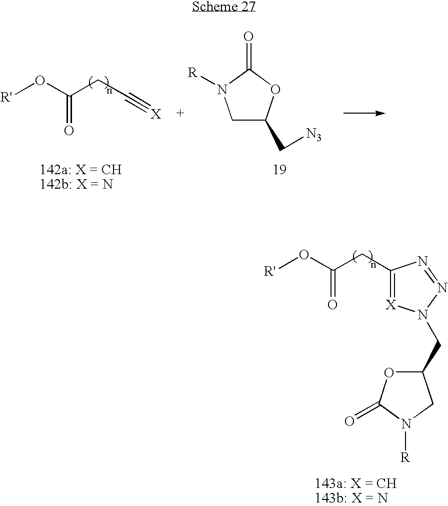

The chemistry illustrated in Scheme 27 can be applied to macrolide systems containing alknynyl or cyano esters, as illustrated in Scheme 28. Here, 6-O-mycaminosyl azithromycin 34a is treated with alkynyl carboxylic acid 144a or cyano carboxylic acid 144b under mild esterification conditions (using a coupling agent such as DCC, EDC, HOBt, etc.) to yield the alkynyl ester 145a or the cyano ester 145b. These esters are then treated with azide 19 to yield via a cycloaddition reaction the triazole 146a or the tetrazole 146b.

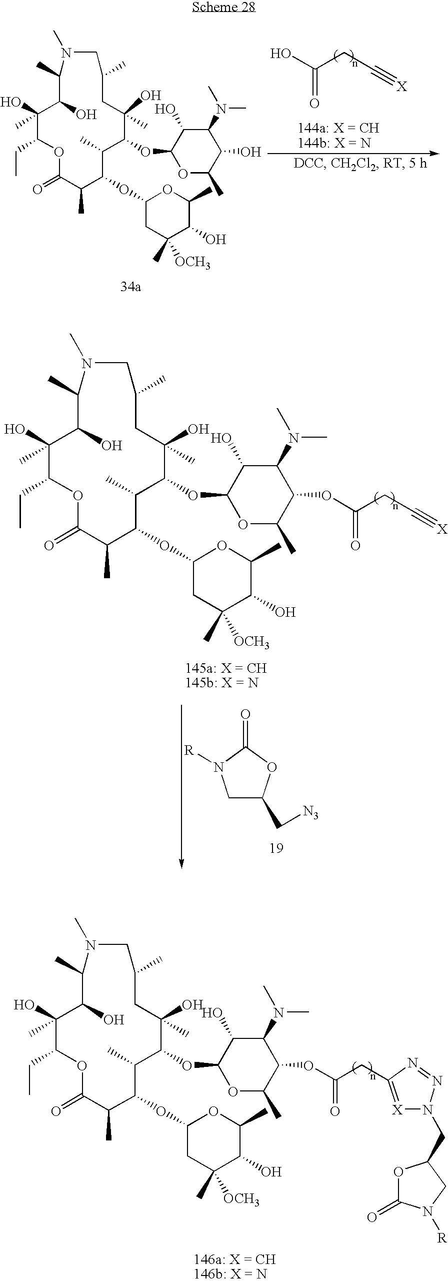

Alternatively, compounds of the present invention containing an ester moiety linking the 5-membered heterocyclic ring to the macrolide can be prepared by first forming the cycloaddition product from an alkynyl or cyano carboxylic acid, and subsequently esterifying with a macrolide. Scheme 29 illustratates how an alkynyl carboxylic acid 144a or a cyano carboxylic acid 144b can be treated with azide 19 to yield the corresponding triazole acid 147a or tetrazole acid 147b, respecitvely.

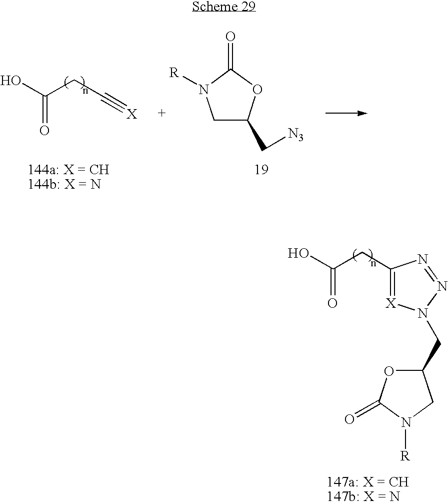

Scheme 29 illustrates the reaction of 6-O-mycaminosyl azithromycin 34a with carboxylic acid 147a or 147b under mild esterification conditions (using a coupling agent such as DCC, EDC, HOBt, etc.) to yield the final product 146a or 146b.

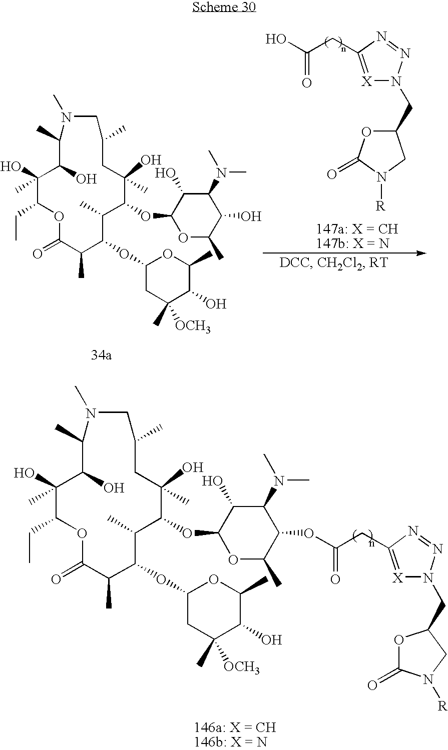

In addition to the foregoing, compounds disclosed in the following publications, patents and patent applications are suitable intermediates for preparation of the compounds of this invention:

Tucker, J. A. et al., J. Med. Chem., 1998, 41, 3727; Gregory, W. A. et al., J. Med. Chem., 1990, 33, 2569; Genin, M. J. et al., J. Med. Chem., 1998, 41, 5144; Brickner, S. J. et al., J. Med. Chem., 1996, 39, 673. Barbachyn, M. R. et al., J. Med. Chem., 1996, 39, 680; Barbachyn, M. R. et al., Bioorg. Med. Chem. Lett., 1996, 6, 1003; Barbachyn, M. R. et al., Bioorg. Med. Chem. Lett., 1996, 6, 1009; Grega, K. C. et al., J. Org. Chem., 1995, 60, 5255; Park, C.-H. et al., J. Med. Chem., 1992, 35, 1156; Yu, D. et al., Bioorg. Med. Chem. Lett., 2002, 12, 857; Weidner-Wells, Wells, M. A. et al., Bioorg. Med. Chem., 2002, 10, 2345; and Cacchi, S. et al., Org. Lett., 2001, [symbol] , 2539. U.S. Pat. Nos. 4,801,600; 4,948, 801; 5,736,545; 6,362,189; 5,523,403; 4,461,773; 5,365,751; 6,124,334; 6,239,152; 5,981,528; 6,194,441; 6,147,197; 6,034,069; 4,990,602; 5,124,269; and 6,271,383. U.S. Patent Application Nos. 2001/0046992, PCT Application and publications WO96/15130; WO95/14684; WO 99/28317; WO 98/01447; WO 98/01446; WO 97/31917; WO 97/27188; WO 97/10223; WO 97/09328; WO 01/46164; WO 01/09107; WO 00/73301; WO 00/21960; WO 01/81350; WO 97/30995; WO 99/10342; WO 99/10343; WO 99/64416; WO 00/232917; and WO 99/64417, European Patent Nos. EP 0312000 B1; EP 0359418 A1; EP 00345627; EP 1132392; and EP 0738726 A1.

### 4. CHARACTERIZATION OF COMPOUNDS OF THE INVENTION

Compounds designed, selected and/or optimized by methods described herein, once produced, may be characterized using a variety of assays known to those skilled in the art to determine whether the compounds have biological activity. For example, the molecules may be characterized by conventional assays, including but not limited to those assays described below, to determine whether they have a predicted activity, binding activity and/or binding specificity.

Furthermore, high-throughput screening may be used to speed up analysis using such assays. As a result, it may be possible to rapidly screen the molecules described herein for activity, for example, as anti-cancer, anti-bacterial, anti-fungal, anti-parasitic or anti-viral agents. Also, it may be possible to assay how the compounds interact with a ribosome or ribosomal subunit and/or are effective as modulators (for example, inhibitors) of protein synthesis using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

(1) Surface Binding Studies. A variety of binding assays may be useful in screening new molecules for their binding activity. One approach includes surface plasmon resonance (SPR) that can be used to evaluate the binding properties of molecules of interest with respect to a ribosome, ribosomal subunit or a fragment thereof.

SPR methodologies measure the interaction between two or more macromolecules in real-time through the generation of a quantum-mechanical surface plasmon. One device, (BIAcore Biosensor® from Pharmacia Biosensor, Piscatawy, N.J.) provides a focused beam of polychromatic light to the interface between a gold film (provided as a disposable biosensor “chip”) and a buffer compartment that can be regulated by the user. A 100 nm thick “hydrogel” composed of carboxylated dextran that provides a matrix for the covalent immobilization of analytes of interest is attached to the gold film. When the focused light interacts with the free electron cloud of the gold film, plasmon resonance is enhanced. The resulting reflected light is spectrally depleted in wavelengths that optimally evolved the resonance. By separating the reflected polychromatic light into its component wavelengths (by means of a prism), and determining the frequencies that are depleted, the BIAcore establishes an optical interface which accurately reports the behavior of the generated surface plasmon resonance. When designed as above, the plasmon resonance (and thus the depletion spectrum) is sensitive to mass in the evanescent field (which corresponds roughly to the thickness of the hydrogel). If one component of an interacting pair is immobilized to the hydrogel, and the interacting partner is provided through the buffer compartment, the interaction between the two components can be measured in real time based on the accumulation of mass in the evanescent field and its corresponding effects of the plasmon resonance as measured by the depletion spectrum. This system permits rapid and sensitive real-time measurement of the molecular interactions without the need to label either component.

(2) Fluorescence Polarization. Fluorescence polarization (FP) is a measurement technique that can readily be applied to protein-protein, protein-ligand, or RNA-ligand interactions in order to derive IC₅₀s and Kds of the association reaction between two molecules. In this technique one of the molecules of interest is conjugated with a fluorophore. This is generally the smaller molecule in the system (in this case, the compound of interest). The sample mixture, containing both the ligand-probe conjugate and the ribosome, ribosomal subunit or fragment thereof, is excited with vertically polarized light. Light is absorbed by the probe fluorophores, and re-emitted a short time later. The degree of polarization of the emitted light is measured. Polarization of the emitted light is dependent on several factors, but most importantly on viscosity of the solution and on the apparent molecular weight of the fluorophore. With proper controls, changes in the degree of polarization of the emitted light depends only on changes in the apparent molecular weight of the fluorophore, which in-turn depends on whether the probe-ligand conjugate is free in solution, or is bound to a receptor. Binding assays based on FP have a number of important advantages, including the measurement of IC₅₀s and Kds under true homogenous equilibrium conditions, speed of analysis and amenity to automation, and ability to screen in cloudy suspensions and colored solutions.

(3) Protein Synthesis. It is contemplated that, in addition to characterization by the foregoing biochemical assays, the compound of interest may also be characterized as a modulator (for example, an inhibitor of protein synthesis) of the functional activity of the ribosome or ribosomal subunit.

Furthermore, more specific protein synthesis inhibition assays may be performed by administering the compound to a whole organism, tissue, organ, organelle, cell, a cellular or subcellular extract, or a purified ribosome preparation and observing its pharmacological and inhibitory properties by determining, for example, its inhibition constant (IC₅₀) for inhibiting protein synthesis. Incorporation of ³H leucine or ³⁵S methionine, or similar experiments can be performed to investigate protein synthesis activity. A change in the amount or the rate of protein synthesis in the cell in the presence of a molecule of interest indicates that the molecule is a modulator of protein synthesis. A decrease in the rate or the amount of protein synthesis indicates that the molecule is a inhibitor of protein synthesis.

Furthermore, the compounds may be assayed for anti-proliferative or anti-infective properties on a cellular level. For example, where the target organism is a microorganism, the activity of compounds of interest may be assayed by growing the microorganisms of interest in media either containing or lacking the compound. Growth inhibition may be indicative that the molecule may be acting as a protein synthesis inhibitor. More specifically, the activity of the compounds of interest against bacterial pathogens may be demonstrated by the ability of the compound to inhibit growth of defined strains of human pathogens. For this purpose, a panel of bacterial strains can be assembled to include a variety of target pathogenic species, some containing resistance mechanisms that have been characterized. Use of such a panel of organisms permits the determination of structure-activity relationships not only in regards to potency and spectrum, but also with a view to obviating resistance mechanisms. The assays may be performed in microtiter trays according to conventional methodologies as published by The National Committee for Clinical Laboratory Standards (NCCLS) guidelines (NCCLS. M7-A5-Methods for Dilution Antimicrobial Susceptibility Tests for Bacteria That Grow Aerobically; Approved Standard-Fifth Edition. NCCLS Document M100-S12/M7 (ISBN 1-56238-394-9)).

### 5. FORMULATION AND ADMINISTRATION

The compounds of the invention may be useful in the prevention or treatment of a variety of human or other animal disorders, including for example, bacterial infection, fungal infections, viral infections, parasitic diseases, and cancer. It is contemplated that, once identified, the active molecules of the invention may be incorporated into any suitable carrier prior to use. The dose of active molecule, mode of administration and use of suitable carrier will depend upon the intended recipient and target organism. The formulations, both for veterinary and for human medical use, of compounds according to the present invention typically include such compounds in association with a pharmaceutically acceptable carrier.

The carrier(s) should be “acceptable” in the sense of being compatible with the other ingredients of the formulations and not deleterious to the recipient. Pharmaceutically acceptable carriers, in this regard, are intended to include any and all solvents, dispersion media, coatings, anti-bacterial and anti-fungal agents, isotonic and absorption delaying agents, and the like, compatible with pharmaceutical administration. The use of such media and agents for pharmaceutically active substances is known in the art. Except insofar as any conventional media or agent is incompatible with the active compound, use thereof in the compositions is contemplated. Supplementary active compounds (identified or designed according to the invention and/or known in the art) also can be incorporated into the compositions. The formulations may conveniently be presented in dosage unit form and may be prepared by any of the methods well known in the art of pharmacy/microbiology. In general, some formulations are prepared by bringing the compound into association with a liquid carrier or a finely divided solid carrier or both, and then, if necessary, shaping the product into the desired formulation.

A pharmaceutical composition of the invention should be formulated to be compatible with its intended route of administration. Examples of routes of administration include oral or parenteral, for example, intravenous, intradermal, inhalation, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Useful solutions for oral or parenteral administration can be prepared by any of the methods well known in the pharmaceutical art, described, for example, in Remington's Pharmaceutical Sciences, (Gennaro, A., ed.), Mack Pub., (1990). Formulations for parenteral administration can also include glycocholate for buccal administration, methoxysalicylate for rectal administration, or citric acid for vaginal administration. The parenteral preparation can be enclosed in ampoules, disposable syringes or multiple dose vials made of glass or plastic. Suppositories for rectal administration also can be prepared by mixing the drug with a non-irritating excipient such as cocoa butter, other glycerides, or other compositions which are solid at room temperature and liquid at body temperatures. Formulations also can include, for example, polyalkylene glycols such as polyethylene glycol, oils of vegetable origin, and hydrogenated naphthalenes. Formulations for direct administration can include glycerol and other compositions of high viscosity. Other potentially useful parenteral carriers for these drugs include ethylene-vinyl acetate copolymer particles, osmotic pumps, implantable infusion systems, and liposomes. Formulations for inhalation administration can contain as excipients, for example, lactose, or can be aqueous solutions containing, for example, polyoxyethylene-9-lauryl ether, glycocholate and deoxycholate, or oily solutions for administration in the form of nasal drops, or as a gel to be applied intranasally. Retention enemas also can be used for rectal delivery.

Formulations of the present invention suitable for oral administration may be in the form of: discrete units such as capsules, gelatin capsules, sachets, tablets, troches, or lozenges, each containing a predetermined amount of the drug; a powder or granular composition; a solution or a suspension in an aqueous liquid or non-aqueous liquid; or an oil-in-water emulsion or a water-in-oil emulsion. The drug may also be administered in the form of a bolus, electuary or paste. A tablet may be made by compressing or molding the drug optionally with one or more accessory ingredients. Compressed tablets may be prepared by compressing, in a suitable machine, the drug in a free-flowing form such as a powder or granules, optionally mixed by a binder, lubricant, inert diluent, surface active or dispersing agent. Molded tablets may be made by molding, in a suitable machine, a mixture of the powdered drug and suitable carrier moistened with an inert liquid diluent.

Oral compositions generally include an inert diluent or an edible carrier. For the purpose of oral therapeutic administration, the active compound can be incorporated with excipients. Oral compositions prepared using a fluid carrier for use as a mouthwash include the compound in the fluid carrier and are applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration may be in the form of a sterile aqueous preparation of the drug that may be in microcrystalline form, for example, in the form of an aqueous microcrystalline suspension. Liposomal formulations or biodegradable polymer systems may also be used to present the drug for both intra-articular and ophthalmic administration.

Formulations suitable for topical administration, including eye treatment, include liquid or semi-liquid preparations such as liniments, lotions, gels, applicants, oil-in-water or water-in-oil emulsions such as creams, ointments or pastes; or solutions or suspensions such as drops. Formulations for topical administration to the skin surface can be prepared by dispersing the drug with a dermatologically acceptable carrier such as a lotion, cream, ointment or soap. Particularly useful are carriers capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used.

For inhalation treatments, inhalation of powder (self-propelling or spray formulations) dispensed with a spray can, a nebulizer, or an atomizer can be used. Such formulations can be in the form of a fine powder for pulmonary administration from a powder inhalation device or self-propelling powder-dispensing formulations. In the case of self-propelling solution and spray formulations, the effect may be achieved either by choice of a valve having the desired spray characteristics (i.e., being capable of producing a spray having the desired particle size) or by incorporating the active ingredient as a suspended powder in controlled particle size. For administration by inhalation, the compounds also can be delivered in the form of an aerosol spray from pressured container or dispenser which contains a suitable propellant, e.g., a gas such as carbon dioxide, or a nebulizer.

Systemic administration also can be by transmucosal or transdermal means. For transmucosal or transdermal administration, penetrants appropriate to the barrier to be permeated are used in the formulation. Such penetrants generally are known in the art, and include, for example, for transmucosal administration, detergents and bile salts. Transmucosal administration can be accomplished through the use of nasal sprays or suppositories. For transdermal administration, the active compounds typically are formulated into ointments, salves, gels, or creams as generally known in the art.

The active compounds may be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

Oral or parenteral compositions can be formulated in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. Furthermore, administration can be by periodic injections of a bolus, or can be made more continuous by intravenous, intramuscular or intraperitoneal administration from an external reservoir (e.g., an intravenous bag).

Where adhesion to a tissue surface is desired the composition can include the drug dispersed in a fibrinogen-thrombin composition or other bioadhesive. The compound then can be painted, sprayed or otherwise applied to the desired tissue surface. Alternatively, the drugs can be formulated for parenteral or oral administration to humans or other mammals, for example, in therapeutically effective amounts, e.g., amounts that provide appropriate concentrations of the drug to target tissue for a time sufficient to induce the desired effect.

Where the active compound is to be used as part of a transplant procedure, it can be provided to the living tissue or organ to be transplanted prior to removal of tissue or organ from the donor. The compound can be provided to the donor host. Alternatively or, in addition, once removed from the donor, the organ or living tissue can be placed in a preservation solution containing the active compound. In all cases, the active compound can be administered directly to the desired tissue, as by injection to the tissue, or it can be provided systemically, either by oral or parenteral administration, using any of the methods and formulations described herein and/or known in the art. Where the drug comprises part of a tissue or organ preservation solution, any commercially available preservation solution can be used to advantage. For example, useful solutions known in the art include Collins solution, Wisconsin solution, Belzer solution, Eurocollins solution and lactated Ringer's solution.

Active compound as identified or designed by the methods described herein can be administered to individuals to treat disorders (prophylactically or therapeutically). In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a drug as well as tailoring the dosage and/or therapeutic regimen of treatment with the drug.

In therapeutic use for treating, or combating, bacterial infections in mammals, the compounds or pharmaceutical compositions thereof will be administered orally, parenterally and/or topically at a dosage to obtain and maintain a concentration, that is, an amount, or blood-level or tissue level of active component in the animal undergoing treatment which will be anti-microbially effective. The term “effective amount” is understood to mean that the compound of the invention is present in or on the recipient in an amount sufficient to elicit biological activity, for example, anti-microbial activity, anti-fungal activity, anti-viral activity, anti-parasitic activity, and/or anti-proliferative activity. Generally, an effective amount of dosage of active component will be in the range of from about 0.1 to about 100, more preferably from about 1.0 to about 50 mg/kg of body weight/day. The amount administered will also likely depend on such variables as the type and extent of disease or indication to be treated, the overall health status of the particular patient, the relative biological efficacy of the compound delivered, the formulation of the drug, the presence and types of excipients in the formulation, and the route of administration. Also, it is to be understood that the initial dosage administered may be increased beyond the above upper level in order to rapidly achieve the desired blood-level or tissue level, or the initial dosage may be smaller than the optimum and the daily dosage may be progressively increased during the course of treatment depending on the particular situation. If desired, the daily dose may also be divided into multiple doses for administration, for example, two to four times per day.

### 6. EXAMPLES

Nuclear magnetic resonance (NMR) spectra were obtained on a Bruker Avance 300 or Avance 500 spectrometer, or in some cases a GE-Nicolet 300 spectrometer. Common reaction solvents were either high performance liquid chromatography (HPLC) grade or American Chemical Society (ACS) grade, and anhydrous as obtained from the manufacturer unless otherwise noted. “Chromatography” or “purified by silica gel” refers to flash column chromatography using silica gel (EM Merck, Silica Gel 60, 230-400 mesh) unless otherwise noted.

### Example 1

### Synthesis of Compound 208

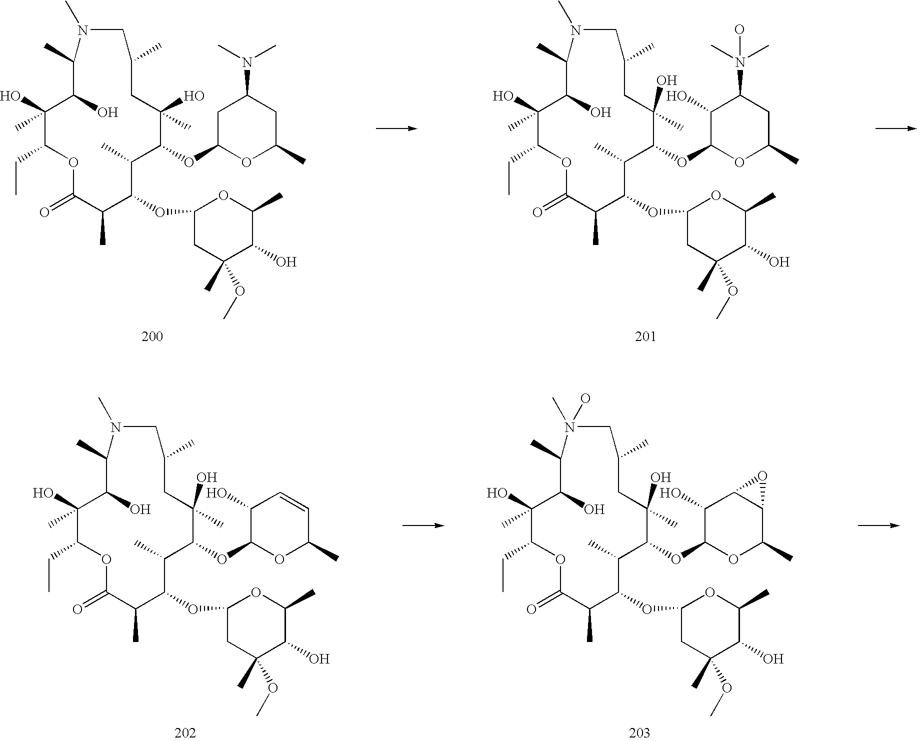 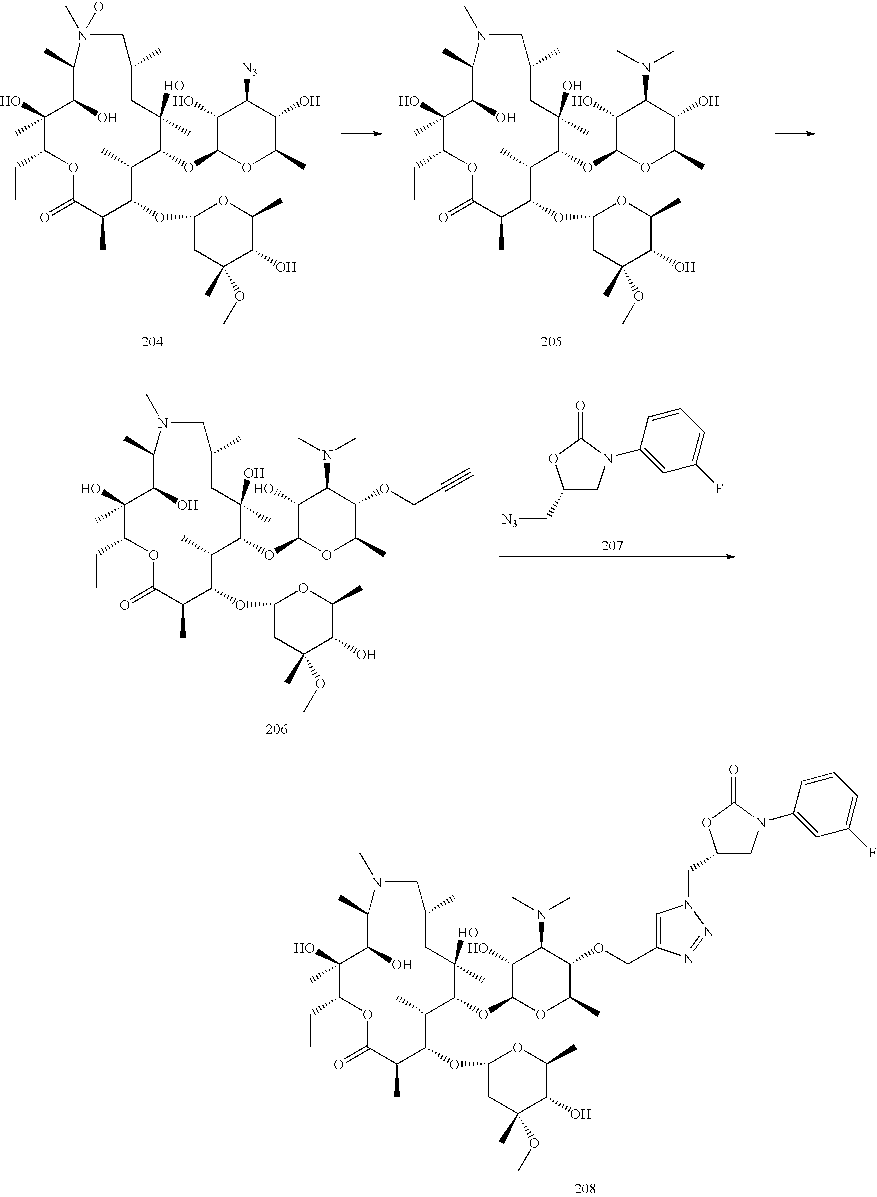 Synthesis of Azithromycin-3′-N-oxide 201

Azithromycin 200 (50 g, 66.8 mmol) was dissolved in enough warm acetone to make 150 mL of solution. This solution was allowed to cool to ambient temperature prior to addition of 40 ml of 30% w/w aqueous H₂O₂. Following a mild exotherm, the solution was allowed to cool to ambient temperature and stirred for 3.5 h. The reaction mixture was diluted to 2 L with CH₂Cl₂ and the resulting gelatinous mixture was stirred vigorously for 1 h to afford a cloudy suspension. This suspension was washed with a 5:1 mixture of saturated aqueous NaHCO₃ and 10% w/v aqueous Na₂S₂O₃ (2×600 mL) and with brine (1×800 mL). The aqueous washes were combined and adjusted to pH 12 with 2N KOH and then further extracted with CH₂Cl₂ (3×300 mL). The combined organic extracts were dried over K₂CO₃, filtered, and concentrated in vacuo. As the volume of the extracts was reduced crystals began to form; when the total volume of the extracts had been reduced to 700 mL the solution was placed in a stoppered flask and stored at room temperature overnight. The solids were collected by vacuum filtration, rinsed with cold ether, and dried under vacuum to afford 34 g of white needle-like crystals. The filtrate was treated as before to yield two additional crops of crystalline product 201 for a total yield of 51 g (66.7 mmol 99%). ¹HNMR (300 MHz, CDCl₃, partial): δ 5.06 (d, J=4 Hz, 1H), 4.69 (d, J=9 Hz, 1H), 4.53 (d, J=7 Hz, 1H), 4.27 (d, J=3 Hz, 1H), 4.11-4.02 (m, 1H), 3.75 (dd, J=10, 7 Hz, 1H), 3.68 (s, 1H), 3.62 (d, J=7 Hz, 1H), 3.46-3.39 (m, 1H), 3.37 (s, 3H), 3.20 (s, 6H), 3.04 (d, J=9 Hz, 1H) 3.07-2.99 (m, 1H), 2.81-2.70 (m, 2H), 2.48 (d, J=11 Hz, 1H), 2.42-2.25 (m, 2H), 2.15-1.84 (m, 2H), 1.78 (d, J=15 Hz, 1H), 1.56 (dd, J=15, 5 Hz, 1H), 1.54-1.40 (m, 1H), 1.29 (d, J=6 Hz, 3H), 1.27 (s, 3H), 1.25 (s, 3H), 1.24 (s, 3H), 1.18 (d, J=7 Hz, 3H), 0.91 (t, J=5 Hz, 3H), 0.86 (t, J=7 Hz, 3H). ¹³CNMR (100 MHz, CDCl₃): δ 178.6, 102.5, 94.9, 78.4, 78.1, 77.8, 76.4, 74.3, 73.4, 72.9, 72.5, 66.9, 65.5, 59.1, 52.0, 49.7, 45.2, 41.8, 36.5, 34.9, 27.5, 26.7., 22.1, 21.6, 21.3, 18.5, 16.5, 15.0, 11.2, 9.0, 7.4. LCMS (ESI) m/z 765.6 (M+H)⁺.

Synthesis of 3′ desdimethylamino-4′-dehydro-azithromycin 202

A 300 mL pear-shaped recovery flask was charged with Azithromycin-3′-N-oxide 201 (35 g, 45.8 mmol) and placed on a rotary evaporator. The pressure was reduced to 0.5 torr and the flask was rotated slowly in an oil bath while the temperature was gradually increased to 175° C. The mixture was held under vacuum at this temperature for 1.5 h then cooled to room temperature and flushed with argon. The resulting tan solid was dissolved in 800 mL of boiling acetonitrile. The solution was allowed to cool slowly to room temperature and then placed in a −20° C. freezer overnight. The solids were collected by vacuum filtration and washed with cold acetonitrile to afford 19.1 g of 202 as off-white crystals. The filtrate was concentrated and the residue treated as above to afford two additional crops of 202 product for a total yield of 27.7 g (39.4 mmol, 86%). ¹HNMR (300 MHz, CDCl₃, partial): δ 5.70-5.49 (m, 2H), 4.95 (d, J=4 Hz, 1H), 4.64 (dd, J=10, 2 Hz, 1H), 4.51 (d, J=7 Hz, 1H), 4.40-4.29 (m, 1H), 4.25 (dd, J=7, 2 Hz, 1H), 4.18-4.05 (m, 2H), 3.68 (d, J=6 Hz, 1H), 3.65-3.59 (m, 2H), 3.28 (s, 3H), 3.03 (dd, J=9, 11 Hz, 1H), 2.85 (p, J=7 Hz, 1H), 2.74 (q, J=7 Hz, 1H), 2.64 (bs, 1H), 2.55-2.40 (m, 3H), 2.35 (s, 3H), 2.30 (d, J=15 Hz, 1H), 2.11-1.83 (m, 5H), 1.55 (dd, J=10, 4 Hz, 1H), 1.55-1.45 (m, 1H), 1.37 (bs, 3H), 1.30 (d, J=6 Hz, 1H), 1.24 (s, 3H), 1.23 (s, 3H), 1.21 (s, 3H), 1.10, (d, J=8 Hz, 1H), 1.07 (s, 3H), 1.00 (d, J=7 Hz, 3H), 0.91 (d, J=7 Hz, 3H), 0.89 (t, J=7 Hz, 3H). ¹³CNMR (100 MHz, CDCl₃): δ 176.3, 130.3, 124.5, 100.8, 94.0, 83.4, 77.7, 76.1, 75.9, 75.6, 73.2, 72.5, 71.7, 71.2, 68.3, 68.2, 67.0, 63.6, 60.1, 47.5, 43.1, 40.6, 38.6, 34.8, 33.1, 25.3, 24.9, 20.0, 19.7, 19.1, 16.2, 14.4, 13.9, 9.36, 7.9, 5.8. LCMS (ESI) m/z 704.5 (M+H)⁺.

Synthesis of 3 desdimethylamino-4]-dehydro-3′,4′-epoxy-9′N-oxo-azithromycin 203

To a methanol solution of 202 (25.0 g, 35.5 mmol in 100 mL) was added mCPBA (20.4 g, 89 mmol). The reaction mire was stirred at room temperature for 14 h at which time an additional 10 g portion of mCPBA was added. The solution was stirred for an additional 4 h, then diluted with 1200 mL CH₂Cl₂ and washed with saturated aqueous NaHCO₃ (2×500 mL) and brine (1×500 mL). The aqueous washes were back-extracted with CH₂Cl₂ (2×500 mL). The combined organic extracts were dried on K₂CO₃, filtered, and concentrated to give a white foam (30.7 g) which was purified by silica gel chromatography (125 mm×6″ column eluted with 7.5% 2N NH₃ in MeOH/CH₂Cl₂) to afford compound 203 as a white solid (25.7 g, 35.0 mmol, 98%). ¹HNMR (300 MHz, CDCl₃): δ 5.10 (d, J=4 Hz, 1H), 5.03 (dd, J=8.4 Hz, 1H), 4.41 (d, J=7 Hz, 1H), 4.38 (d, J=3 Hz, 1H), 4.22 (bs, 1H), 4.11 (d, J=11 Hz, 1H), 4.04-3.92 (m, 1H), 3.52 (d, J=8 Hz, 1H), 3.48-3.23 (m, 4H), 3.34 (s, 3H), 3.10 (d, J=9 Hz, 1H), 2.99 (t, J=10 Hz, 1H), 2.88 (bs, 3H), 2.72-2.60 (m, 2H), 2.58 (dd, J=4.7 Hz, 1H), 2.54-2.42 (m, 3H), 2.31 (d, J=15 Hz, 1H), 2.29 (d, J=10 Hz, 1H), 2.08-1.80 (m, 2H), 1.57 (d, J=7 Hz, 1H), 1.54-1.38 (m, 3H), 1.37 (s, 3H), 1.28 (d, J=6 Hz, 3H), 1.26 (d, J=6 Hz, 3H), 1.23, (s, 3H), 1.18-1.10 (m, 6H), 1.04 (s, 3H), 0.96 (d, J=6 Hz, 3H), 0.90 (t, J=7 Hz, 3H). LCMS (ESI) m/z 779.6 (M+H)⁺.

Synthesis of 3′β-azido-4′α-hydroxy-9′N-oxo-3′-desdimethylamino-azithromycin 204

Epoxide 203 (20.0 g, 27.2 mmol) was dissolved in 88 mL of 10:1 DMSO-H₂O to which was added NaN₃ (17.7 g, 270 mmol) and Mg(ClO₄).8H₂O (13.5 g, 40.8 mmol). The mixture was stirred under argon at 85° C. for 16 h then cooled to room temperature and poured into saturated aqueous NaHCO₃ (1L) and extracted with CH₂Cl₂ (5×500 mL). The combined organic extracts were dried over K₂CO₃, filtered, and concentrated to afford a white foam (29 g). This material was dissolved in hot CH₃CN (1.2L) and allowed to sit overnight at room temperature. The solids were filtered from the solution and rinsed with additional CH₃CN. The 8.7 g of crystalline solid thus obtained was confirmed by NMR and x-ray analysis to be pure 3′α-hydroxy-4′β-azido-9′N-oxo-3′-desdimethylamino-azithromycin formed by addition of the azide at the 4′ carbon of the epoxide. The mother liquors were concentrated and the residue again dissolved in boiling CH₃CN from which a second 3.0 g crop of the undesired isomer was obtained in pure form. The mother liquors, now enriched in the desired product 204, were concentrated and the residue purified by silica gel chromatography (50 mm×8″ column eluted with 0-8% 2N NH₃ in MeOH/CH₂Cl₂) to afford an additional 2.9 g of the earlier-eluting 4′β-azide along with the title compound 204 (6.5 g, 8.3 mmol, 31%). ¹HNMR (300 MHz, CDCl₃): δ5.01 (d, J=4 Hz, 1H), 4.95 (dd, J=8.4 Hz, 11), 4.40 (d, J=7 Hz, 1H), 4.31 (d, J=4 Hz, 1H), 4.15 (bs, 1H), 4.05 (d, J=12 Hz, 1H), 3.97 (d, J=7 Hz, 1H), 3.92 (dd, J=9, 3 Hz, 1H), 3.66 (d, J=7 Hz, 1H), 3.35 (bs, 1H), 3.35-3.31 (m, 1H), 3.25 (s, 3H), 3.23-3.15 (m, 1H), 3.05 (d, J=4 Hz, 1H), 2.91 (t, J=7 Hz, 1H), 2.81 (bs, 3H), 2.63 (bs, 1H), 2.56-2.36 (m, 4H), 2.33-2.26 (m, 1H), 2.23 (d, J=15 Hz, 1H), 1.98-1.73 (m, 2H), 1.48 (d, J=7 Hz, 1H), 1.45-1.27 (m, 4H), 1.25 (s, 3H), 1.23 (d, J=7 Hz, 3H), 1.17, (d, J=6 Hz, 1H), 1.13 (s, 3H), 1.07 (d, J=7 Hz, 3H), 1.05 (d, J=6 Hz, 3H), 0.99 (s, 3H), 0.89 (d, J=7 Hz, 3H), 0.82 (t, J=7 Hz, 3H). ¹³CNMR (100 MHz, CDCl₃): δ 177.7, 99.6, 94.5, 83.6, 78.2, 77.5, 76.7, 74.8, 74.5, 73.9, 72.8, 70.9, 69.4, 68.0, 65.0, 59.2, 55.9, 52.3, 49.2, 46.0, 43.8, 40.6, 34.8, 30.9, 27.0, 25.2, 22.8, 22.5, 21.7, 18.8, 17.8, 16.6, 14.9, 11.7, 9.9, 9.2. LCMS (ESI) m/z 736.6 (M+H)⁺.

Synthesis of 4′α-hydroxy-azithromycin 205

A heavy-walled pressure tube was charged with an ethanol solution of 204 (1.73 g, 2.22 mmol in 20 mL) and 20% palladium on charcoal (0.14 g containing 50% H₂O). The reaction mixture was stirred under an H₂ atmosphere (15 psig) at room temperature for 14 h at which time 2 mL 37% aqueous CH₂O, 1 mL HCO₂H, and an additional 50 mg Pd on C were added. The hydrogen pressure was increased to 30 psig and stirring was continued for 24 h. At which time an additional 100 mg charge of Pd was added and the H₂ pressure was increased to 90 psig. After an additional 24 h at this pressure the reaction mixture was purged with argon, filtered, diluted with 100 mL toluene, and concentrated in vacuo to afford 1.9 g of a colorless glass. The crude product was purified by silica gel chromatography (25 mm×6″ column eluted with 7% 2N NH₃ in MeOH/CH₂Cl₂) to afford compound 205 as a white solid (0.78 g, 1.0 mmol, 45%). ¹HNMR (300 MHz, CDCl₃): δ 4.92 (d, J=4 Hz, 1H), 4.61 (dd, J=10, 2 Hz, 1H), 4.42 (d, J=7 Hz, 1H), 4.18 (dd, J=7, 2 Hz, 1H), 4.11-4.02 (m, 1H), 3.65-3.60 (m, 2H), 3.57 (dd, J=10, 7 Hz, 1H), 3.33-3.23 (m, 1H), 3.28 (s, 3H), 3.05-2.95 (m, 2H), 2.86-2.62 (m, 3H), 2.52-2.38 (m, 2H), 2.47 (s, 6H), 2.35-2.27 (m, 2H), 2.32 (s, 3H), 2.10-2.62 (m, 5H), 1.55 (dd, J=15, 5 Hz, 1H), 1.52-1.40 (m, 1H), 1.34 (s, 3H), 1.32 (d, J=7 Hz, 1H), 1.28 (d, J=6 Hz 3H), 1.22 (s, 3H), 1.19 (d, J=6 Hz, 3H), 1.09 (d, J=6 Hz, 3H), 1.04, (s, 3H), 0.97 (d, J=7 Hz, 3H), 0.90 (d, J=6 Hz, 3H), 0. 0.88 (t, J=7 Hz, 3H). LCMS (ESI) m/z 765.5 (M+H)⁺.

Synthesis of 4′α-propargyloxy-azithromycin 206

To a solution of 500 mg 205 (0.65 mmol) and 200 μL propargyl bromide (2.0 mmol) in CH₂Cl₂ (5 mL) was added 1 mL 50% w/w KOH(aq.) and 20 mg of Bu₄N⁺Br⁻. This mixture was stirred vigorously at room temperature for 4 h, then an additional charge of propargyl bromide (100 uL) and Bu₄N⁺Br⁻ (20 mg) was added. After stirring for 2 h more, the reaction mixture was diluted with CH₂Cl₂ (100 mL) and water (50 mL). The aqueous layer was separated and extracted with CH₂Cl₂ (2×50 mL). The combined organic extracts were dried on K₂CO₃, filtered, and concentrated to give 520 mg of an off-white foam. The crude product contains a mixture of starting material, mono-alkylated products (4″-propargyloxy-4′α-hydroxy-azithromycin and 2′-propargyloxy-4′α-hydroxy-azithromycin along with the desired product), and smaller amounts of bis-alkylated products. The desired product was recovered by preparative thin layer chromatography (plates developed with 7.5% 2N NH₃ in MeOH/CH₂Cl₂) to afford compound 206 as a white solid (48 mg, 60 μmol, 9.1%). ¹HNMR (300 MHz, CDCl₃): δ 4.95 (d, J=4 Hz, 1H), 4.60 (dd, J=10, 2 Hz, 1H), 4.42 (d, J=7 Hz, 1H), 4.38-4.33 (m, 2H), 4.29 (m, 1H), 4.23 (dd, J=6, 2 Hz, 1H), 4.05-3.96 (m, 1H), 3.65-3.58 (m, 2H), 3.35-3.25 (m, 1H), 3.28 (s, 3H), 3.18 (t, J=9 Hz, 1H), 2.99 (d, J=9 Hz, 3H), 2.87-2.75 (m, 1H), 2.73-2.60 (m, 1H), 2.2.54-2.45 (m, 1H), 2.48 (t, J=2 Hz, 1H), 2.35-2.20 (m, 2H), 2.32 (s, 3H), 2.10-1.80 (m, 4H), 1.75 (d, J=15 Hz, 1H), 1.55 (dd, J=15, 4 Hz, 1H), 1.55-1.40 (m, 1H) 1.34-1.15 (m, 18H), 1.08 (d, J=6 Hz, 3H), 1.04, (s, 3H), 1.01 (d, J=7 Hz, 3H), 0.92-0.81 (m, 6H). LCMS (ESI) m/z 803.5 (M+H)⁺.

Synthesis of Compound 208

A 1 dram vial was charged with alkyne 206 (24 mg, 30 μmol), azide 207 (14 mg, 60 μmol) and THF (300 uL). The solution was degassed by alternately exposing to high vacuum and flushing with argon. CuI was added and the reaction stirred at room temperature for 3 h. The entire reaction mixture was placed on a preparative thin layer chromatography plate and eluted twice with 5% 2N NH₃ in MeOH/CH₂Cl₂ to afford compound 208 as a white solid (18 mg, 17 μmol, 58%). ¹HNMR (300 MHz, CDCl₃): δ 7.72 (bs, 1H), 7.35-7.20 (m, 2H), 7.10-7.0 (m, 1H), 6.82-6.73 (td, J=8, 2 Hz, 1H), 5.10-4.55 (m, 6H), 4.42 (d, J=7 Hz, 1H), 4.2-3.7 (m, 5H), 3.65-3.50 (m, 2H), 3.31-3.15 (m, 2H), 3.25 (s, 3H), 2.95 (t, J=10 Hz, 1H), 2.79-2.60 (m, 2H), 2.45 (bs, 6H), 2.28 (bs, 3H), 2.15-1.75 (m, 3H), 1.75 (d, J=15 Hz, 1H), 1.49, (dd, J=15, 4 Hz, 1H), 1.45-1.32 (m, 1H), 1.30-1.10 (m, 15H), 1.06 (d, J=6 Hz, 3H), 0.9-0.78 (m, 6H). LCMS (ESI) m/z 520.4 (M+2H)²⁺, 1040.6 (M+H)⁺.

### Example 2

### Synthesis of Compound 210

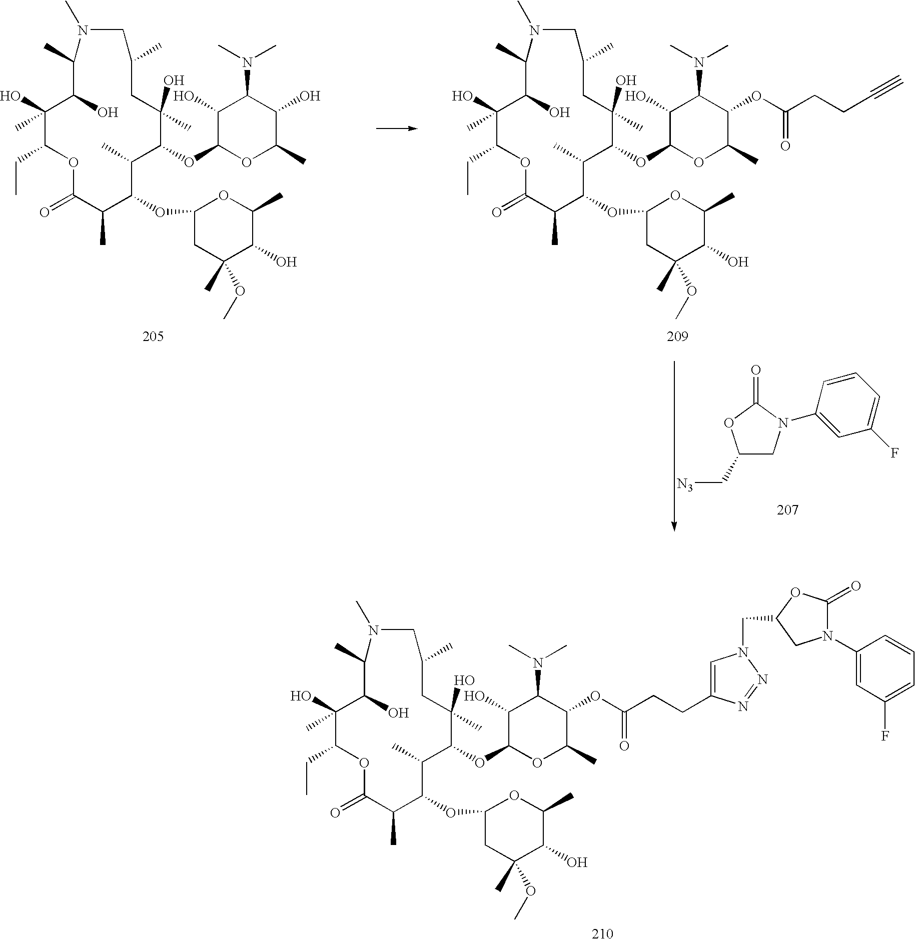 Synthesis of Compound 209

A solution of 4′α-hydroxy-azithromycin 205 (50 mg, 0.066 mmol), 4-pentynoic acid (6.4 mg, 0.066 mmol) and dicyclohexyl carbodiimide (14.8 mg, 0.072 mmol) in CH₂Cl₂ (1.5 ml) was stirred at ambient temperature for 7 h. The solution was filtered through a cotton plug, concentrated and purified by flash chromatography over silica gel (CH₂Cl₂:MeOH:NH₄OH=20:1:0.05) to yield 35 mg of 209. LCMS (ESI) m/z 423.4 (M+2H)²⁺, 845.6 (M+H)⁺.

Synthesis of Compound 210

To a mixture of compound 209 (29 mg, 0.034 mmol), azide 207 (9.7 mg, 0.041) and CuI (3.27 mg, 0.017 mmol) was added THF (3 mL) and Hunig's base (0.050 mL). The solution was degassed with argon, and the resulting mixture was stirred under argon atmosphere at ambient temperature for 1 h. Another portion of azide 207 (9.7 mg, 0.041 mmol) was added and the reaction mixture was stirred for additional 1 h. The reaction mixture was poured into a saturated solution of NH₄Cl (25 mL) containing NH₄OH (3 mL) and stirred for 10 minutes. The resulting mixture was extracted with CH₂Cl₂ (3×50 mL), dried (anhydrous Na₂SO₄), concentrated and purified by flash chromatography over silica gel (CH₂Cl₂:MeOH:NH₄OH=20:1:0.05) to yield 15 mg of compound 210. LCMS (ESI) m/z 541.5 (M+2H)²⁺, 1081.8 (M+H)⁺.

### INCORPORATION BY REFERENCE

The entire disclosure of each of the patent documents and scientific articles referred to herein is incorporated by reference herein for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A compound having the formula: 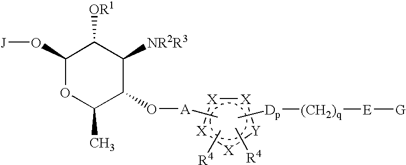
or a pharmaceutically acceptable salt, ester, or prodrug thereof,
wherein:
—O-A is selected from the group consisting of: 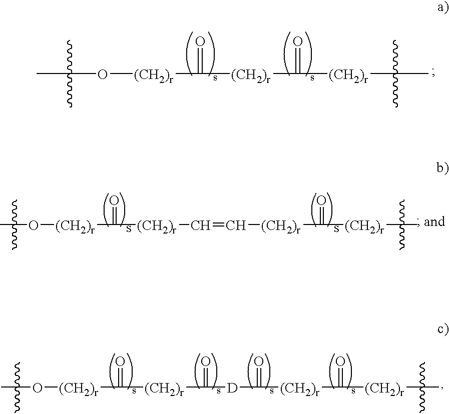
wherein
r, at each occurrence, independently is 0, 1, 2 3, or 4, and
s, at each occurrence, independently is 0 or 1;
X, at each occurrence, independently is carbon, carbonyl, or nitrogen, provided at least one X is carbon;
Y is carbon, nitrogen, oxygen, or sulfur;
D is selected from the group consisting of:
O, S, NR⁵, C═O, C═S, C═NOR⁵, SO, and SO₂;
E-G is selected from the group consisting of 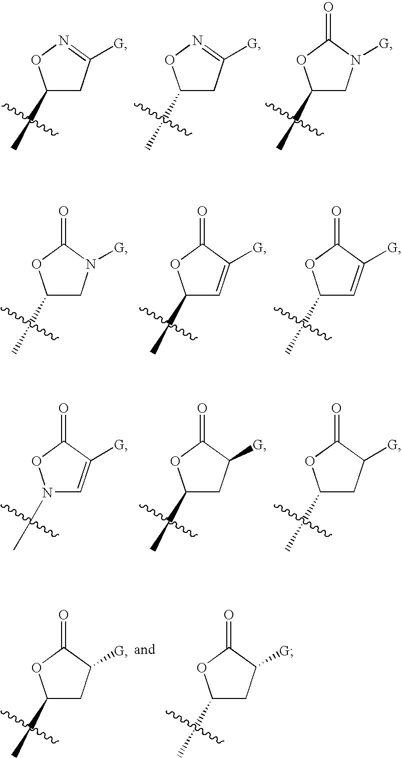
G is selected from the group consisting of: 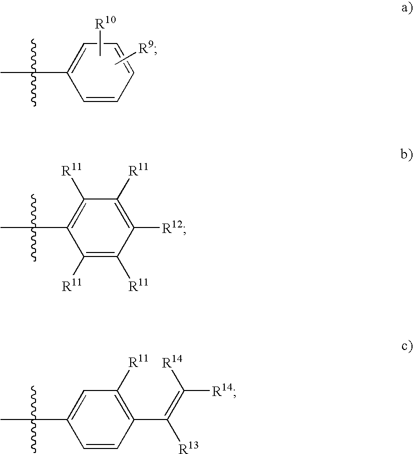
d) 3-14 membered saturated, unsaturated, or aromatic heterocycle containing one or more heteroatoms selected from the group consisting of nitrogen, oxygen, and sulfur, and optionally substituted with one or more R⁴ groups;
e) C₃₋₁₄ saturated, unsaturated, or aromatic carbocycle, optionally substituted with one or more R⁴ groups;
f) C₁₋₈ alkyl,
g) C₂₋₈ alkenyl,
h) C₂₋₈ alkynyl,
i) C₁₋₈ alkoxy,
j) C₁₋₈ alkylthio,
k) C₁₋₈ acyl,
l) S(O)ₜR⁵; and
m) hydrogen,
wherein any of f)-k) optionally is substituted with
i) one or more R⁴ groups;
ii) 3-14 membered saturated, unsaturated, or aromatic heterocycle containing one or more heteroatoms selected from the group consisting of nitrogen, oxygen, and sulfur, and optionally substituted with one or more R⁴ groups; or
iii) C₃₋₁₄ saturated, unsaturated, or aromatic carbocycle, optionally substituted with one or more R⁴ groups;
J is selected from the group consisting of:
a) H, b) Lᵤ-C₁₋₆ alkyl, c) Lᵤ-C₂₋₆ alkenyl, d) Lᵤ-C₂₋₆ alkynyl, e) Lᵤ-C₃₋₁₄ saturated, unsaturated, or aromatic carbocycle, f) Lᵤ-(3-14 membered saturated, unsaturated, or aromatic heterocycle comprising one or more heteroatoms selected from the group consisting of nitrogen, oxygen, and sulfur), and g) macrolide,
wherein
L is selected from the group consisting of —C(O)—, —C(O)O—, and —C(O)NR⁵—,
u is 0 or 1, and
any of b)-f) optionally is substituted with one or more R⁴ groups;
R¹, R², and R³ are independently selected from the group consisting of:
a) H, b) Lᵤ-C₁₋₆ alkyl, c) Lᵤ-C₂₋₆ alkenyl, d) Lᵤ-C₂₋₆ alkynyl, e) Lᵤ-C₃₋₁₄ saturated, unsaturated, or aromatic carbocycle, f) Lᵤ-(3-14 membered saturated, unsaturated, or aromatic heterocycle comprising one or more heteroatoms selected from the group consisting of nitrogen, oxygen, and sulfur), g) Lᵤ-(saturated, unsaturated, or aromatic 10-membered bicyclic ring system optionally containing one or more heteroatoms selected from the group consisting of nitrogen, oxygen, and sulfur), and h) Lᵤ-(saturated, unsaturated, or aromatic 13-membered tricyclic ring system optionally containing one or more heteroatoms selected from the group consisting of nitrogen, oxygen, and sulfur),
wherein
L is selected from the group consisting of —C(O)—, —C(O)O—, and —C(O)NR⁷,
u is 0 or 1, and
any of b)-h) optionally is substituted with one or more R⁴ groups;
alternatively, R², and R³, taken together with the nitrogen atom to which they are bonded, form a 5-7 membered saturated, unsaturated, or aromatic heterocycle optionally containing one or more additional atoms selected from the group consisting of nitrogen, oxygen, and sulfur, and optionally substituted with one or more R⁴ groups;
R⁴, at each occurrence, independently is selected from the group consisting of:
a) F, b) Cl, c) Br, d) I, e) ═O, f) ═S, g) ═NR⁵, h) ═NOR⁵, i) ═NS(O)ₜR⁵, j) ═N—NR⁵R⁵, k) —CF₃, l) —OR⁵, m) —CN, n) —NO₂, o) —NR⁵R⁵, p) —NR⁵OR⁵, q) —C(O)R⁵, r) —C(O)OR⁵, s) —OC(O)R⁵, t) —C(O)NR⁵R⁵, u) —NR⁵C(O)R⁵, v) —OC(O)NR⁵R⁵, w) —NR⁵C(O)OR⁵, x) —NR⁵C(O)NR⁵R⁵, y) —C(S)R⁵, z) —C(S)OR⁵, aa) —OC(S)R⁵, bb) —C(S)NR⁵R⁵, cc) —NR⁵C(S)R⁵, dd) —OC(S)NR⁵R⁵, ee) —NR⁵C(S)OR⁵, ff) —NR⁵C(S)NR⁵R⁵, gg) —C(═NR⁵)R⁵; hh) —C(═NR⁵)OR⁵, ii) —OC(═NR⁵)R⁵, jj) —C(═NR⁵)NR⁵R⁵, kk) —NR⁵C(═NR⁵)R⁵, ll) —OC(═NR⁵)NR⁵R⁵, mm) —NR⁵C(═NR⁵)OR⁵, nn) —NR⁵C(═NR⁵)NR⁵R⁵, oo) —NR⁵C(═NR⁵)NR⁵R⁵, pp) —S(O)ₜR⁵, qq) —SO₂NR⁵R⁵, rr) —S(O)ₜN═R⁵, and ss) R⁵;
R⁵, at each occurrence, independently is selected from the group consisting of:
a) H, b) Lᵤ-C₁₋₆ alkyl, c) Lᵤ-C₂₋₆ alkenyl, d) Lᵤ-C₂₋₆ alkynyl, e) Lᵤ-C₃₋₁₄ saturated, unsaturated, or aromatic carbocycle, f) Lᵤ-(3-14 membered saturated, unsaturated, or aromatic heterocycle comprising one or more heteroatoms selected from the group consisting of nitrogen, oxygen, and sulfur), g) Lᵤ-(saturated, unsaturated, or aromatic 10-membered bicyclic ring system optionally containing one or more heteroatoms selected from the group consisting of nitrogen, oxygen, and sulfur), and h) Lᵤ-(saturated, unsaturated, or aromatic 13-membered tricyclic ring system optionally containing one or more heteroatoms selected from the group consisting of nitrogen, oxygen, and sulfur),
wherein
L is selected from the group consisting of —C(O)—, —C(O)O—, and —C(O)NR⁸,
u is 0 or 1, and
any of b)-h) optionally is substituted with one or more R⁶ groups;
alternatively, two R⁵ groups, taken together with the atom or atoms to which they are bonded, form i) a 5-7 membered saturated, unsaturated, or aromatic carbocycle, or ii) a 5-7 membered saturated, unsaturated, or aromatic heterocycle containing one or more atoms selected from the group consisting of nitrogen, oxygen, and sulfur,
wherein i)-ii) optionally is substituted with one or more R⁶ groups;
R⁶, at each occurrence, independently is selected from the group consisting of:
a) F, b) Cl, c) Br, d) I, e) ═O, f) ═S, g) ═NR⁷, h) ═NOR⁷, i) ═NS(O)ₜR⁷, j) ═N—NR⁷R⁷, k) —CF₃, l) —OR⁷, m) —CN, n) —NO₂, o) —NR⁷R⁷, p) —NR⁷OR⁷, q) —C(O)R⁷, r) —C(O)OR⁷, s) —OC(O)R⁷, t) —C(O)NR⁷R⁷, u) —NR⁷C(O)R⁷, v) —OC(O)NR⁷R⁷, w) —NR⁷C(O)OR⁷, x) —NR⁷C(O)NR⁷R⁷, y) —C(S)R⁷, z) —C(S)OR⁷, aa) —OC(S)R⁷, bb) —C(S)NR⁷R⁷, cc) —NR⁷C(S)R⁷, dd) —OC(S)NR⁷R⁷, ee) —NR⁷C(S)OR⁷, ff) —NR⁷C(S)NR⁷R⁷, gg) —C(═NR⁷)R⁷; hh) —C(═NR⁷)OR⁷, ii) —OC(═NR⁷)R⁷, jj) —C(═NR⁷)NR⁷R⁷, kk) —NR⁷C(═NR⁷)R⁷, ll) —OC(═NR⁷)NR⁷R⁷, mm) —NR⁷C(═NR⁷)OR⁷, nn) —NR⁷C(═NR⁷)NR⁷R⁷, oo) —NR⁷C(═NR⁷)NR⁷R⁷, pp) —S(O)ₜR⁷, qq) —SO₂NR⁷R⁷, rr) —S(O)ₜN═R⁷, and ss) R⁷;
R⁷, at each occurrence, independently is selected from the group consisting of:
a) H, b) Lᵤ-C₁₋₆ alkyl, c) Lᵤ-C₂₋₆ alkenyl, d) Lᵤ-C₂₋₆ alkynyl, e) Lᵤ-C₃₋₁₄ saturated, unsaturated, or aromatic carbocycle, f) Lᵤ-(3-14 membered saturated, unsaturated, or aromatic heterocycle comprising one or more heteroatoms selected from the group consisting of nitrogen, oxygen, and sulfur), g) Lᵤ-(saturated, unsaturated, or aromatic 10-membered bicyclic ring system optionally containing one or more heteroatoms selected from the group consisting of nitrogen, oxygen, and sulfur), and h) Lᵤ-(saturated, unsaturated, or aromatic 13-membered tricyclic ring system optionally containing one or more heteroatoms selected from the group consisting of nitrogen, oxygen, and sulfur),
wherein
L is selected from the group consisting of C(O), C(O)O, and C(O)NR⁷,
u is 0 or 1, and
any of b)-h) optionally is substituted with one or more moieties selected from the group consisting of:
R⁸, F, Cl, Br, I, —CF₃, —OR⁸, —SR⁸, —CN, —NO₂, —NR⁸R⁸, —C(O)R⁸, —C(O)OR⁸, —OC(O)R⁸, —C(O)NR⁸R⁸, —NR⁸C(O)R⁸, —OC(O)NR⁸R⁸, —NR⁸C(O)OR⁸, —NR⁸C(O)NR⁸R⁸, —C(S)R⁸, —C(S)OR⁸, —OC(S)R⁸, —C(S)NR⁸R⁸, —NR⁸C(S)R⁸, —OC(S)NR⁸R⁸, —NR⁸C(S)OR⁸, —NR⁸C(S)NR⁸R⁸, —NR⁸C(NR⁸)NR⁸R⁸, —SO₂NR⁸R⁸, and —S(O)ₜR⁸;
alternatively, two R⁷ groups, taken together with the atom or atoms to which they are bonded, form i) a 5-7 membered saturated, unsaturated, or aromatic carbocycle, or ii) a 5-7 membered saturated, unsaturated, or aromatic heterocycle containing one or more atoms selected from the group consisting of nitrogen, oxygen, and sulfur;
R⁸, at each occurrence, independently is selected from the group consisting of:
a) H, b) Lᵤ-C₁₋₆ alkyl, c) Lᵤ-C₂₋₆ alkenyl, d) Lᵤ-C₂₋₆ alkynyl, e) Lᵤ-C₃₋₁₄ saturated, unsaturated, or aromatic carbocycle, i) Lᵤ-(3-14 membered saturated, unsaturated, or aromatic heterocycle comprising one or more heteroatoms selected from the group consisting of nitrogen, oxygen, and sulfur), g) Lᵤ-(saturated, unsaturated, or aromatic 10-membered bicyclic ring system optionally containing one or more heteroatoms selected from the group consisting of nitrogen, oxygen, and sulfur), and h) Lᵤ-(saturated, unsaturated, or aromatic 13-membered tricyclic ring system optionally containing one or more heteroatoms selected from the group consisting of nitrogen, oxygen, and sulfur),
wherein
L is selected from the group consisting of —C(O)—, —C(O)O—, and —C(O)NH—, —C(O)N(C₁₋₆ alkyl)-and
u is 0 or 1;
R⁹ is R⁴;
R¹⁰ is R⁴;
alternatively, R⁹ and R¹⁰, taken together with the atoms to which they are bonded, form i) a 5-7 membered saturated, unsaturated, or aromatic carbocycle, or ii) a 5-7 membered saturated, unsaturated, or aromatic heterocycle containing one or more atoms selected from the group consisting of nitrogen, oxygen, and sulfur,
wherein i)-ii) optionally is substituted with one or more R⁴ groups;
R¹¹ is R⁴;
alternatively, two R¹¹ groups, taken together with the atoms to which they are bonded, form i) a 5-7 membered saturated, unsaturated, or aromatic carbocycle, or ii) a 5-7 membered saturated, unsaturated, or aromatic heterocycle containing one or more atoms selected from the group consisting of nitrogen, oxygen, and sulfur,
wherein i)-ii) optionally is substituted with one or more R⁴ groups;
R¹² is R⁵;
alternatively, R¹² and one R¹¹ group, taken together with the atoms to which they are bonded, form i) a 5-7 membered saturated, unsaturated, or aromatic carbocycle, or ii) a 5-7 membered saturated, unsaturated, or aromatic heterocycle containing one or more atoms selected from the group consisting of nitrogen, oxygen, and sulfur,
wherein i)-ii) optionally is substituted with one or more R⁴ groups;
R¹³ is R⁴;
R¹⁴ is R⁴;
alternatively, any R¹³ and any R⁴, taken together with the atoms to which they are bonded, form i) a 5-7 membered saturated, unsaturated, or aromatic carbocycle, or ii) a 5-7 membered saturated, unsaturated, or aromatic heterocycle containing one or more atoms selected from the group consisting of nitrogen, oxygen, and sulfur,
wherein i)-ii) optionally is substituted with one or more R⁴ groups;
p is 0 or 1;
q is 0 or 1; and
t, at each occurrence, independently is 0, 1, or 2.

2. The compound according to claim 1 having the formula: 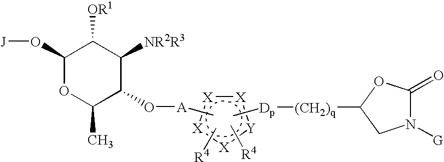
wherein A, D, G, J, R¹, R², R³, R⁴, X, Y, p, and q are as defined in claim 1.

3. The compound according to claim 1 having the formula: 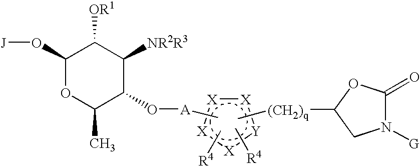
wherein
O-A is selected from the group consisting of:
O—(CH₂)ᵣ, O—C(O), and O—C(O)—(CH₂)ᵣ;
r is 1, 2, 3, or 4;
J is a macrolide; and
G, R¹, R², R³, R⁴, X, Y, and q are as defined in claim 1.

4. The compound according to claim 3 having the formula:

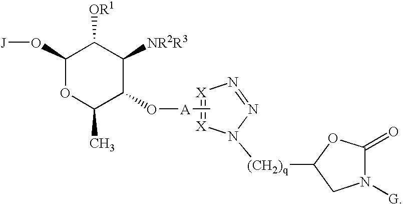

5. The compound according to claim 4 having the formula:

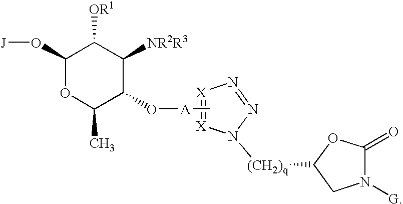

6. The compound according to claim 5 having the formula:

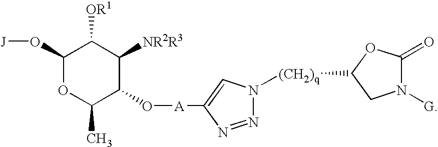

7. The compound according to claim 1, wherein G has the formula: 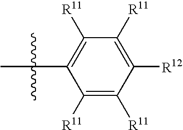
wherein R¹¹ and R¹² are as defined in claim 1.

8. The compound according to claim 7, wherein G has the formula:

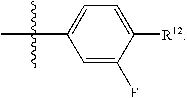

9. The compound according to claim 8, wherein R¹² is H.

10. The compound according to claim 8, wherein R¹² has the formula: 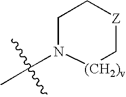
wherein
Z is selected from the group consisting of O, NR⁵, and S(O)ₜ; and
v is 0, 1, 2, or 3.

11. The compound according to claim 10, wherein Z is O and v is 1.

12. The compound according to claim 7, wherein R¹² is —C(O)CH₃.

13. The compound according to claim 7, wherein R¹² has the formula: 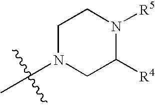
wherein R⁴ and R⁵ are as defined in claim 1.

14. The compound according to claim 13, wherein R⁵ is —C(O)—CH₂—OH.

15. The compound according to claim 13, wherein R⁴ is H.

16. The compound according to claim 1, having the formula: 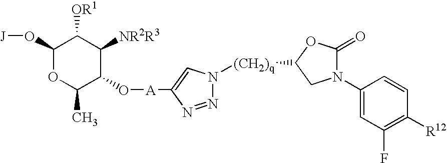
wherein
O-A is selected from the group consisting of:
O—(CH₂)ᵣ, O—C(O), and O—C(O)—(CH₂)ᵣ;
r is 1, 2, 3, or 4;
J is a macrolide; and
R¹, R², R³, R¹², and q are as defined in claim 1.

17. The compound according to claim 16, wherein R¹² is H.

18. The compound according to claim 16, wherein R¹² is

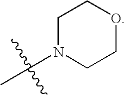

19. The compound according to claim 1, wherein J is a macrolide.

20. The compound according to claim 19, wherein the macrolide is selected from the group consisting of: 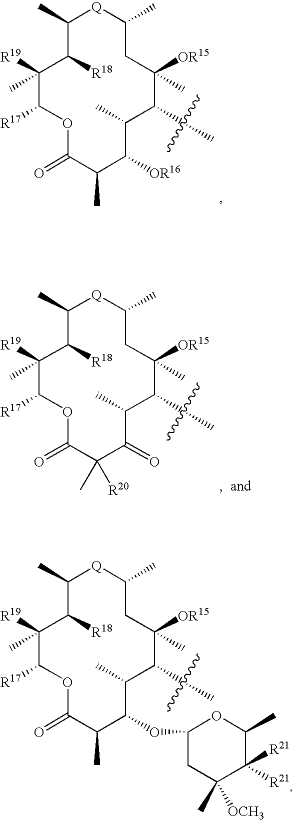
and pharmaceutically acceptable salts, esters and prodrugs thereof, wherein
Q is selected from the group consisting of:
—NR⁵CH₂—, —CH₂—NR⁵—, —C(O), —C(═NR⁵)—, —C(═NOR⁵)—, —C(═N—NR⁵R⁵), —CH(OR⁵), and —CH(NR⁵R⁵)—;
R¹⁵ and R¹⁶ independently are selected from the group consisting of R⁵ and a hydroxy protecting group;
alternatively R¹⁵ and R¹⁶, taken together with the atoms to which they are bonded, form: 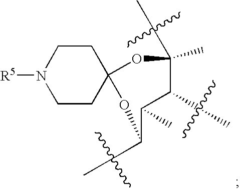
R¹⁷ is selected from the group consisting of:
a) C₁₋₆ alkyl, b) C₂₋₆ alkenyl, and c) C₂₋₆ alkynyl;
wherein any of a)-c) optionally is substituted with one or more moieties selected from the group consisting of
i) —OR⁵, ii) C₃₋₁₄ saturated, unsaturated, or aromatic carbocycle, and iii) 3-14 membered saturated, unsaturated, or aromatic heterocycle containing one or more atoms selected from the group consisting of nitrogen, oxygen, and sulfur,
wherein any of ii)-iii) optionally is substituted with one or more R⁴ groups;
R¹⁸ is selected from the group consisting of:
a) —OR¹⁵, b) C₁₋₆ alkyl, c) C₂₋₆ alkenyl, d) C₂₋₆ alkynyl, e) —C(O)R⁵, and f) —NR⁵R⁵,
wherein any of b)-d) optionally is substituted with one or more R⁴ groups;
alternatively, R¹⁵ and R¹⁸, taken together with the atoms to which they are bonded, form: 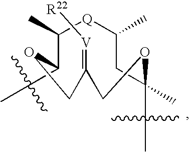
wherein
V is CH or N, and
R²² is —OR⁵, or R⁵;
R¹⁹ is —OR¹⁵;
alternatively, R¹⁸ and R¹⁹, taken together with the atoms to which they are bonded, form a 5-membered ring by attachment to each other through a linker selected from the group consisting of:
—OC(R⁴)(R⁴)O—, —OC(O)O—, —OC(O)NR⁵—, —NR⁵C(O)O, —OC(O)NOR⁵—, —N(OR⁵)C(O)O—, —OC(O)N—NR⁵R⁵—, —N(NR⁵R⁵)C(O)O—, —OC(O)CHR⁵—, —CHR⁴C(O)O—, —OC(S)O—, —OC(S)NR⁵—, —NR⁵C(S)O—, —OC(S)NOR⁵, —N(OR⁵)C(S)O—, —OC(S)N—NR⁵R⁵—, —N(NR⁵R⁵)C(S)O—, —OC(S)CHR⁴—, and —CHR⁴C(S)O—;
alternatively, Q, R¹⁸, and R¹⁹, taken together with the atoms to which they are bonded, form: 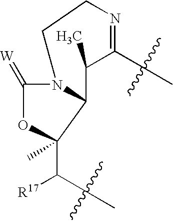
wherein
W is O, NR⁵, or NOR⁵;
R²⁰ is selected from the group consisting of:
H, F, Cl, Br, and C₁₋₆ alkyl;
R²¹, at each occurrence, independently is selected from the group consisting of:
R⁵, —OR¹⁵, and —NR⁵R⁵;
alternatively, two R²¹ groups taken together are ═O, ═N—OR⁵, or ═N—NR⁵R⁵.

21. The compound according to claim 1, wherein J is selected from the group consisting of:

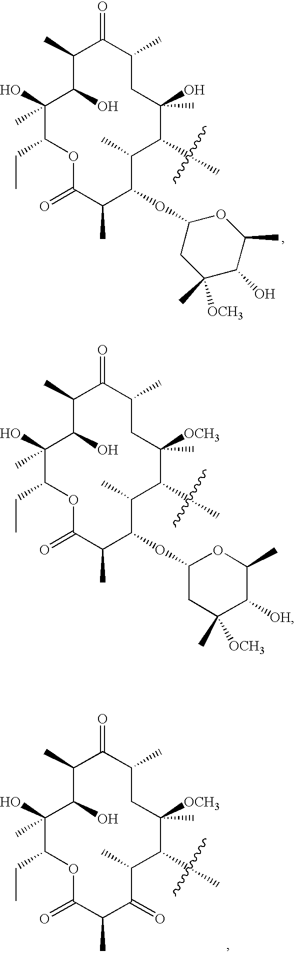

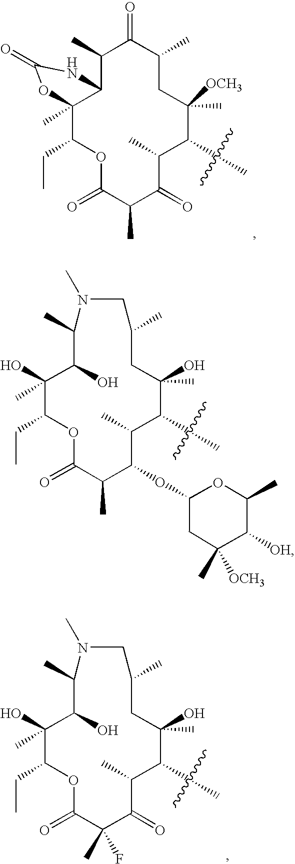

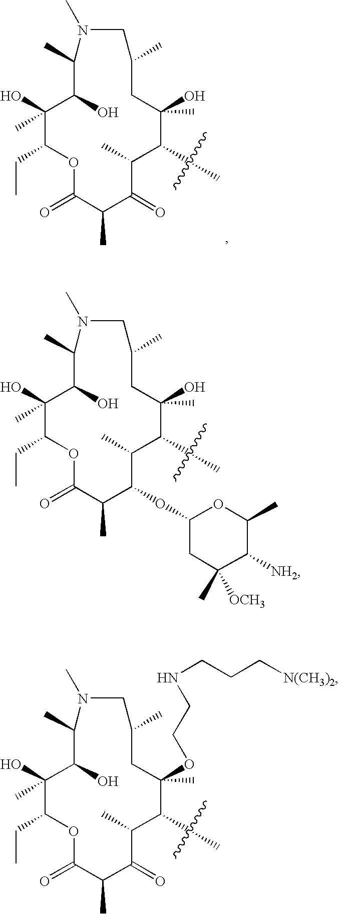

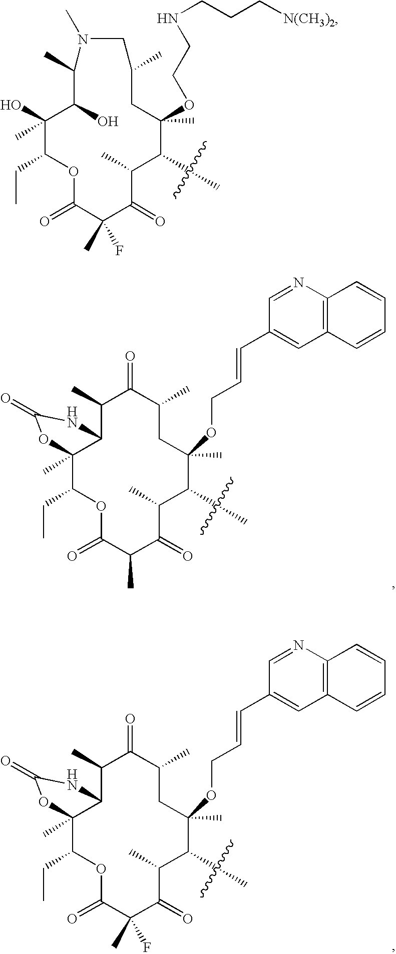

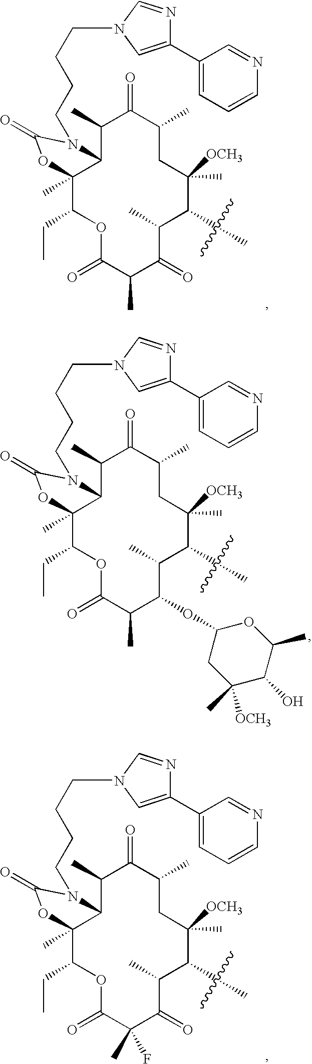

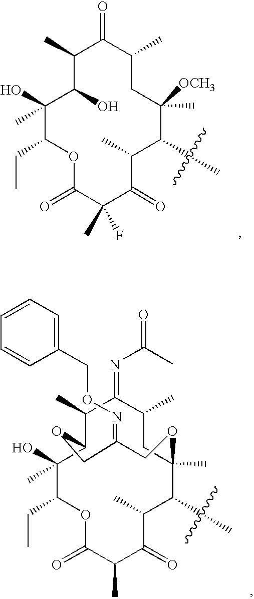

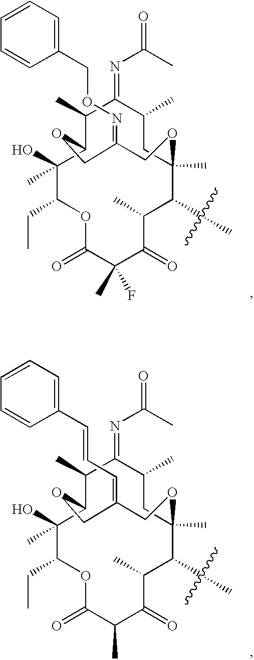

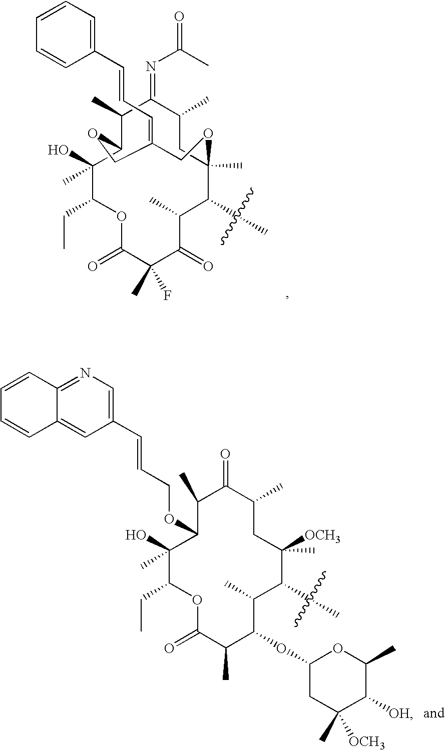

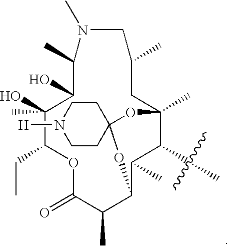

22. The compound according to claim 1, wherein J is:

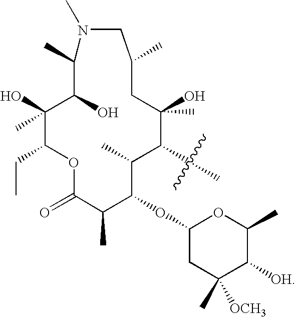

23. The compound according to claim 1, wherein:
R¹ is H;
R² is methyl; and
R³ is methyl.

24. The compound according to claim 1, wherein:
R¹ is H;
R² is H; and
R³ is methyl.

25. A compound having the structure selected from the group consisting of: 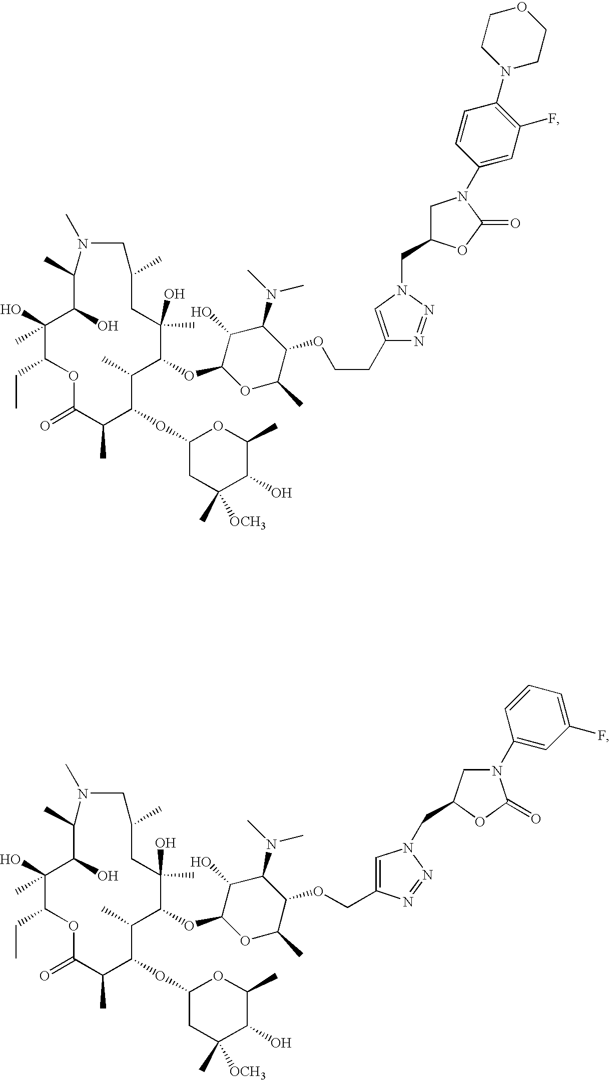 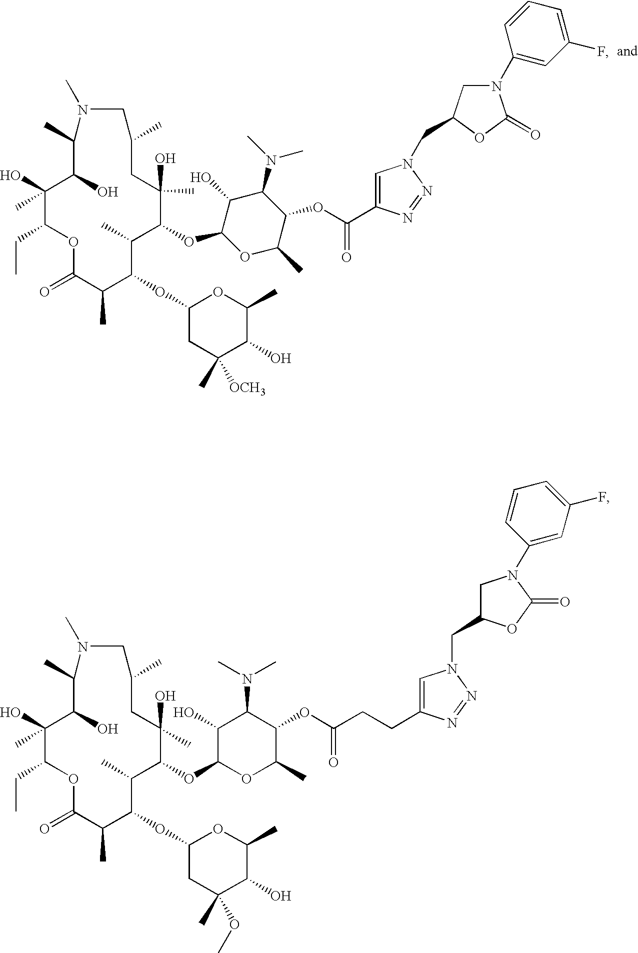 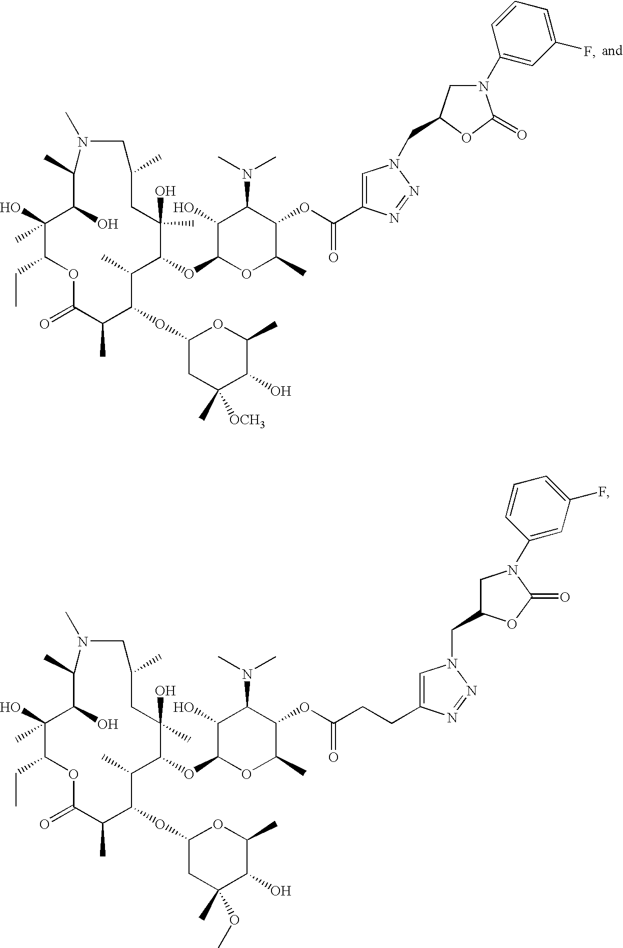
or a pharmaceutically acceptable salt, ester, or prodrug thereof.

26. A pharmaceutical composition comprising a compound according to claim 1 and a pharmaceutically acceptable carrier.

27. A method of treating a microbial infection in a mammal comprising administering to the mammal an effective amount of a compound according to claim 1.

28. A method of treating a fungal infection in a mammal comprising administering to the mammal an effective amount of a compound according to claim 1.

29. A method of treating a parasitic disease in a mammal comprising administering to the mammal an effective amount of a compound according to claim 1.

30. A method of treating a proliferative disease in a mammal comprising administering to the mammal an effective amount of a compound according to claim 1.

31. A method of treating a viral infection in a mammal comprising administering to the mammal an effective amount of a compound according to claim 1.

32. A method of treating an inflammatory disease in a mammal comprising administering to the mammal an effective amount of a compound according to claim 1.

33. A method of treating a gastrointestinal motility disorder in a mammal comprising administering to the mammal an effective amount of a compound according to claim 1.

34. The method according to any one of claims 27-33 wherein the compound is administered orally, parentally, or topically.

35. A method of synthesizing a compound according to claim 1.

36. A medical device containing a compound according to claim 1.

37. The medical device according to claim 36, wherein the device is a stent.
